(19) United States
(12) Patent Application Publication
(10) Pub. No.: US 2003/0092767 A1
(43) Pub. Date: May 15, 2003

(54) Combination therapy for the treatment of inflammatory and respiratory diseases

(76) Inventor: William Louis Macias, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/149,365

(22) PCT Filed: Dec. 22, 2000
(86) PCT No.: PCT/US00/34262

(51) Int. Cl. A61K 31/22

(52) U.S. Cl. 514/546

(57) ABSTRACT

A pharmaceutical composition for the treatment of Inflammatory Disease or Respiratory Disease in mammals, which comprises, as active ingredients, a neutrophil elastase inhibitor and an sPLA₂ inhibitor.

### FIELD OF THE INVENTION

This invention relates to the field of medicine and specifically to the treatment of Inflammatory Diseases and Respiratory Diseases.

### BACKGROUND OF THE INVENTION

Diseases of the respiratory system and inflammatory system present special problems for effective treatment. In particular, it is desirable to discover more effective treatments for diseases of the lower respiratory tract including the trachea, bronchi, and lungs. The greatest need is for new therapeutic agents to treat lung diseases or inflammatory diseases.

Present therapies for lower respiratory diseases and inflammatory diseases are often only partially effective or are not suitable for extended use.

Lung diseases have been treated with neutrophil elastase inhibitors. For example, clinical trials have been conducted with the compound, Sivelestat, a neutrophil elastase inhibitor, (product of Ono Pharmaceutical Company, CAS No. 127373-66-4) for treatment of various lung disorders.

Inflammatory diseases such as sepsis also present special problems, particularly in situations where the patient is experiencing organ failure and/or antibiotics have been ineffective in arresting the septic condition.

The structure and physical properties of human non-pancreatic secretory phospholipase A₂ (hereinafter called, “sPLA₂”) has been thoroughly described in two articles, namely, “Cloning and Recombinant Expression of Phospholipase A2 Present in Rheumatoid Arthritic Synovial Fluid” by Seilhamer, Jeffrey J.; Pruzanski, Waldemar; Vadas Peter; Plant, Shelley; Miller, Judy A.; Kloss, Jean; and Johnson, Lorin K.; The Journal of Biological Chemistry, Vol. 264, No. 10, Issue of April 5, pp. 5335-5338, 1989; and “Structure and Properties of a Human Non-pancreatic Phospholipase A₂” by Kramer, Ruth M.; Hession, Catherine; Johansen, Berit; Hayes, Gretchen; McGray, Paula; Chow, E. Pingchang; Tizard, Richard; and Pepinsky, R. Blake; The Journal of Biological Chemistry, Vol. 264, No. 10, Issue of April 5, pp. 5768-5775, 1989; the disclosures of which are incorporated herein by reference.

It is believed that sPLA₂ is a rate limiting enzyme in the arachidonic acid cascade which hydrolyzes membrane phospholipids. Thus, it is important to develop compounds which inhibit sPLA₂ mediated release of fatty acids (e.g., arachidonic acid). Such compounds are of value in general treatment of Inflammatory Diseases.

It is desirable to create novel and more effective therapies for the treatment of respiratory diseases and inflammatory diseases.

### SUMMARY OF THE INVENTION

It is a discovery of this invention that respiratory diseases are prevented or treated in an advantageous or superior manner by a combination therapy using (i) a neutrophil elastase inhibitor, and (ii) an sPLA₂ inhibitor.

The combination therapy of an sPLA₂ inhibitor with an neutrophil elastase inhibitor synergistically improves treatment and prevention of Respiratory Diseases and Inflammatory Diseases in the human body. Without being bound by any theory of operation, it is believed that both essential ingredients

In particular, for treatment of lung diseases, without being bound by any theory of operation, it is believed that the neutrophil elastase inhibitor and the sPLA₂ inhibitor act synergistically to prevent degradation of surfactant damage in the lungs.

This invention is a pharmaceutical composition comprising:

a neutrophil elastase inhibitor, and

an sPLA₂ inhibitor.

This invention is also a method of treating or preventing respiratory diseases by administering to a mammal in need thereof a therapeutically effective amount of (a) a neutrophil elastase inhibitor and a therapeutically effective amount of (b) an sPLA₂ inhibitor; wherein (a) and (b) are both administered within a therapeutically effective interval.

Without being bound by any theory of operation, it is believed that the combination of an sPLA2 inhibitor and a neutrophil elastase inhibitor (with optional Activated Protein C co-agent) may be particularly effective in the treatment of diseases associated with surfactant dysfunction such as respiratory distress syndrome in the new born, acute lung injury and/or acute respiratory distress syndrome. Surfactant is composed of both lipid and protein and its beneficial physiologic functions can be interfered with by degradation of either component. sPLA2 degrades the lipid component of surfactant while neutrophil elastase degrades the protein component of surfactant. The combination of both the sPLA2 and neutrophil elastase is synergistically better at maintaining surfactant function.

### DETAILED DESCRIPTION OF THE INVENTION

I. Definitions:

For purposes of the present invention, as disclosed and claimed herein, the following terms are as defined below.

Respiratory Diseases—exemplified by lower respiratory diseases such as systemic inflammatory response syndrome, asthma, emphysema, bronchitis, acute lung injury, acute respiratory distress syndrome, idiopathic pulmonary fibrosis, pneumonia, pulmonary edema, pulmonary obstructive disease, endotoxin induced lung damage, non-cell lung cancer, and multiple organ failure resulting from any of the above pathologic processes.

Inflammatory Diseases—refers to diseases such as inflammatory bowel disease, sepsis, septic shock, acute respiratory distress syndrome, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, juvenile arthropathy or juvenile ankylosing spondylitis, reactive arthropathy, infectious or post-infectious arthritis, gonoccocal arthritis, tuberculous arthritis, viral arthritis, fungal arthritis, syphilitic arthritis, Lyme disease, arthritis associated with “vasculitic syndromes”, polyarteritis nodosa, hypersensitivity vasculitis, Luegenec's granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (typing), miscellaneous forms of arthritis, neuropathic joint disease (charco and joint), hemarthrosis (hemarthrosic), Henoch-Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, and multiple organ failure resulting from any of the preceding pathologic processes.

The phrase “therapeutically effective amount” is an amount of (a) neutophil elastase inhibitor or an amount of (b) an sPLA₂ inhibitor which is effective in preventing or treating Respiratory Diseases or Inflammatory Diseases.

The phrase “therapeutically effective interval” is a period of time beginning when one of either (a) the neutophil elastase inhibitor or (b) an sPLA₂ inhibitor is administered to a mammal and ending at the limit of the beneficial effect in preventing or ameliorating the Respiratory or Inflammatory Disease or associated organ failure of (a) or (b).

The phrase “therapeutically effective combination”, used in the practice of this invention, means administration of both (a) neutrophil elastase inhibitor and (b) an sPLA₂ inhibitor, either simultaneously or separately.

The term, “Active Ingredient” as used herein refers to a combination of (a) neutrophil elastase inhibitor and (b) an sPLA₂ inhibitor co-present in a pharmaceutical formulation for the delivery of a treatment regimen that applies this invention.

The term, “injectable liquid carrier” refers to a liquid medium containing either or both of (a) neutrophil elastase inhibitor, or (b) an sPLA₂ inhibitor; wherein (a) and (b) are independently dissolved, suspended, dispersed, or emulsified in the liquid medium.

sPLA₂—secretary phospholipase A₂

sPLA₂ inhibitor—means a compound which inhibits sPLA₂ mediated release of fatty acid.

sepsis—Sepsis is defined as a systemic inflammatory response to infection, associated with and mediated by the activation of a number of host defense mechanisms including the cytokine network, leukocytes, and the complement and coagulation/fibrinolysis systems (Mesters et al., Blood 88:881-886, 1996). Disseminated intravascular coagulation (DIC), with widespread deposition of fibrin in the microvasculature of various organs, is an early manifestation of sepsis/septic shock. DIC is an important mediator in the development of the multiple organ failure syndrome and contributes to the poor prognosis of patients with septic shock (Fourrier et al., Chest 101:816-823, 1992). “sepsis” includes severe sepsis, septic shock, septisemia, and related disease states.

The term, “injectable liquid carrier” refers to a liquid medium containing either or both of (a) sPLA2 inhibitor, or (b) an sPLA₂ inhibitor; wherein (a) and (b) are independently dissolved, suspended, dispersed, or emulsified in the liquid medium.

Other defined chemical terms:

alkyl—a straight or branched chain monovalent hydrocarbon radical such as methyl, ethyl, n-propyl, isopropyl, n-butyl, tertiary butyl, isobutyl, sec-butyl, n-pentyl, and n-hexyl.

alkenyl—a straight chain or branched monovalent hydrocarbon group having the stated number range of carbon atoms, and typified by groups such as vinyl, propenyl, crotonyl, isopentenyl, and various butenyl isomers.

hydrocarbyl—an organic group containing only carbon and hydrogen.

halo—fluoro, chloro, bromo, or iodo.

heterocyclic radical—radicals derived from monocyclic or polycyclic, saturated or unsaturated, substituted or unsubstituted heterocyclic nuclei having 5 to 14 ring atoms and containing from 1 to 3 hetero atoms selected from the group consisting of nitrogen, oxygen or sulfur. phenylpyridinyl, benzylpyridinyl, pyrimidinyl, phenylpyrimidinyl, pyrazinyl, 1,3,5-triazinyl, quinolinyl, phthalazinyl, quinazolinyl, morpholino, thiomorpholino, homopiperazinyl, tetrahydrofuranyl, tetrahydropyranyl, oxacanyl, 1,3-dioxolanyl, 1,3-dioxanyl, 1,4-dioxanyl, tetrahydrothiopheneyl, pentamethylenesulfadyl, 1,3-dithianyl, 1,4-dithianyl, 1,4-thioxanyl, azetidinyl, hexamethyleneiminium, heptamethyleneiminium, piperazinyl and quinoxalinyl.

carbocyclic radical—a radical derived from a saturated or unsaturated, substituted or unsubstituted 5- to 14-membered organic nucleus whose ring forming atoms (other than hydrogen) are solely carbon atoms. Typical carbocyclic radicals are cycloalkyl, cycloalkenyl, phenyl, naphthyl, norbornanyl, bicycloheptadienyl, tolulyl, xylenyl, indenyl, stilbenyl, terphenylyl, diphenylethylenyl, phenyl-cyclohexenyl, acenaphthylenyl, and anthracenyl, biphenyl, bibenzylyl and related bibenzylyl homologues represented by the formula (bb),

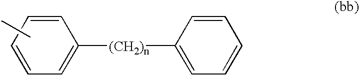

where n is a number from 1 to 8.

non-interfering substituent—radicals suitable for substitution at positions 4, 5, 6, and/or 7 on the indole nucleus (as hereinafter depicted in Formula I) and radical(s) suitable for substitution on the heterocyclic radical and carbocyclic radical as defined above. Illustrative non-interfering radicals are C₁-C₆ alkyl, C₂-C₆ alkenyl, C₂-C₆ alkenyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkenyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₁-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8. acidic group—an organic group which when attached to an indole nucleus, through suitable linking atoms (hereinafter defined as the “acid linker”), acts as a proton donor capable of hydrogen bonding. Illustrative of an acidic group are the following:

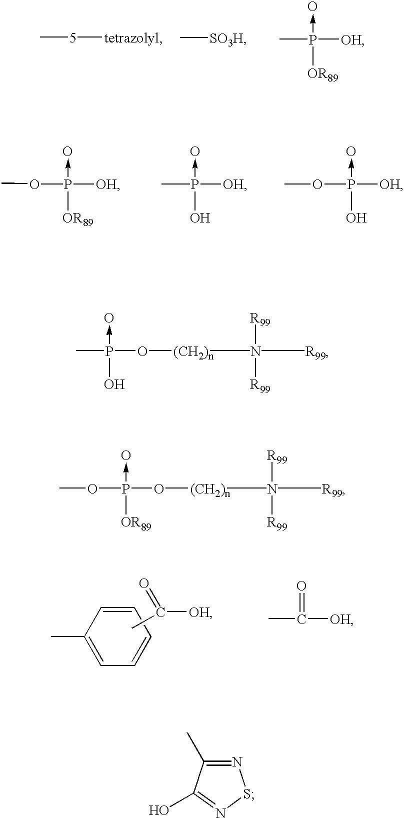

where n is 1 to 8, R₈₉ is a metal or C₁-C₁₀ alkyl, and R₉₉ is hydrogen or C₁-C₁₀ alkyl.

acid linker—a divalent linking group symbolized as, -(Lₐ)-, which has the function of joining the 4 or 5 position of the indole nucleus to an acidic group in the general relationship:

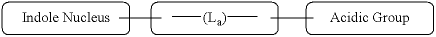

acid linker length—the number of atoms (excluding hydrogen) in the shortest chain of the linking group -(Lₐ)- that connects the 4 or 5 position of the indole nucleus with the acidic group. The presence of a carbocyclic ring in -(Lₐ)- counts as the number of atoms approximately equivalent to the calculated diameter of the carbocyclic ring. Thus, a benzene or cyclohexane ring in the acid linker counts as 2 atoms in calculating the length of -(Lₐ)-. Illustrative acid linker groups are;

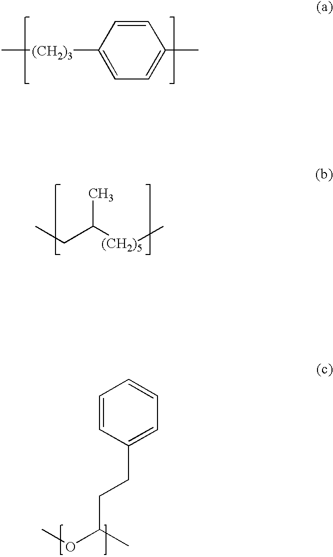

wherein, groups (a), (b), and (c) have acid linker lengths of 5, 7, and 2, respectively.

amine—primary, secondary and tertiary amines.

alkylene chain of 1 or 2 carbon atoms—the divalent radicals, —CH₂—CH₂— and —CH₂—. pharmaceutically acceptable—the carrier, diluent or excipient must be compatible with the other ingredients of the formulation and not deleterious to the recipient thereof.

The term, “carbazole sPLA₂ inhibitors” includes sPLA₂ inhibitors having either a carbazole or a tetrahydrocarbazole nucleus.

II. Preparation of the Neutrophil Elastase Inhibitor Ingredient of the Invention.

The compositions and method of treatment of this invention use compounds known to be active as neutrophil elastase inhibitors. Preferred neutrophil elastase inhibitors are those disclosed in U.S. Pat. No. 5,017,610; 5,336,681; and 5,403,850; the disclosures of which are incorporated herein by reference. These patents also teach suitable method of making their respective inhibitors.

The neutrophil elastase inhibitors most preferred in the practice of this invention are those disclosed in U.S. Pat. No. 5,403,850. In particular, preferred inhibitors are those corresponding to formula (I)

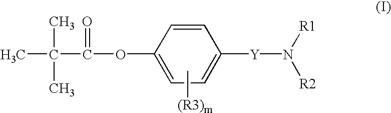

wherein Y represents sulfonyl (—SO₂—) or carbonyl;

(i) R1 and R2 which may be the same or different, each represent

(1) hydrogen,

(2) an alkyl of up to 16 carbon atoms or an alkyl of up to 16 carbon atoms substituted by carboxy,

(3) a group of the formula:

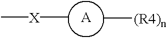

wherein

X represents a single-bond, sulfonyl (—SO₂—), an alkylene of up to 4 carbon atoms, or an alkylene of up to 4 carbon atoms substituted by —COOH or benzyloxy-carbonyl

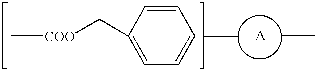

represents a carbocyclic ring or a heterocyclic ring, n represents an integer of 1 to 5,

R4 which may be the same or different represents,

(1) hydrogen or an alkyl group of up to 8 carbon atoms,

(2) an alkoxy of up to 14 carbon atoms,

(3) an alkylthio of up to 6 carbon atoms,

(4) hydroxy, halogen, nitro or trihalomethyl,

(5) a group of the formula: —NR41R42 wherein R41 and R42, which may be the same or different, each represents hydrogen or alkyl of up to 4 carbon atoms,

(6) tetrazole,

(7) sulfonic acid (—SO₃H) or hydroxymethyl (—CH₂OH),

(8) a group of the formula: —SO₂NR41R42 wherein R41 and R42 have the same meanings as described hereinbefore,

(9) a group of the formula: -Z41-COOR43 wherein Z41 represents a single-bond, an alkylene of up to 4 carbon atoms, or an alkenylene of from 2 to 4 carbon atoms, R43 represents hydrogen, an alkyl of up to 4 carbon atoms or benzyl,

(10) a group of the formula: —CONR41R42 wherein R41 and R42 have the same meanings as described hereinbefore,

(11) a group of the formula: —COO-Z42COOR43 wherein Z42 represents an alkylene of up to 4 carbon atoms, R43 represents hydrogen or an alkyl of up to 4 carbon atoms,

(12) a group of the formula: —COO-Z42-CONR41R42 wherein Z42, R41 and R42 have the same meanings as described hereinbefore,

(13) a group of the formula: —OCO-R45 wherein R45 represents an alkyl of up to 8 carbon atoms or p-guanidinophenyl,

(14) a group of the formula: —CO-R46 wherein R46 represents an alkyl of up to 4 carbon atoms,

(15) a group of the formula: —O-Z43-COOR45 wherein Z43 represents an alkylene of up to 6 carbon atoms, R45 represents a hydrogen atom, an alkyl group of up to 8 carbon atoms or a p-guanidinophenyl group,

(16) a group of the formula:

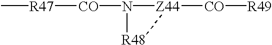

wherein —N-Z44-CO represents an amino acid residue, R48 represents hydrogen or alkyl of up to 4 carbon atoms, and R49 represents hydroxy, alkoxy of up to 4 carbon atoms, amino unsubstituted or substituted by one or two alkyls of up to 4 carbon atoms, carbamoylmethoxy unsubstituted or substituted by one or two alkyls of up to 4 carbon atoms at nitrogen of carbamoyl, R47 represents a single-bond or an alkyl of up to 4 carbon atoms, or

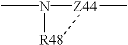

represents a heterocyclic ring containing 3 to 6 carbon atoms and R47 and R49 each has the same meaning as described hereinbefore,

(ii) R1, R2 and nitrogen bonded to R1 and R2 together represent a heterocyclic ring containing at least one nitrogen and substituted by —COOH, or an unsubstituted heterocyclic ring containing at least one nitrogen, R3 represents

(1) hydrogen,

(2) hydroxy,

(3) an alkyl of up to 6 carbon atoms,

(4) halogen,

(5) an alkoxy of up to 4 carbon atoms,

(6) an acyloxy of 2 to 5 carbon atoms, m represents an integer of up to 4, with the proviso that (1) when R1 and R2 represent hydrogen atom or alkyl group of up to 16 carbon atoms, and R3 represents a hydrogen atom or an alkyl group of up to 6 carbon atoms, Y represents carbonyl (—CO—),

and that (2) the compounds wherein one of R1 and R2 represents hydrogen or an alkyl group of up to 16 carbon atoms or 2-carboxyethyl and the other of R1 and R2 represents a group of the formula:

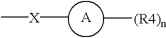

wherein X has the same meaning as described hereinbefore,

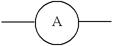

represents a pyridine or pyrrole ring, n represents an integer of 1 or 2, R4 which may be the same or different represents a hydrogen, an alkyl group of up to 8 carbon atoms or a group of the formula: —Z41-COOR43 wherein Z41 and R43 have the same meaning as described hereinbefore, m represents an integer of 1 or 2 and Y and R3 have the same meaning as described hereinbefore, are excluded, or pharmaceutically acceptable salts thereof.

Preferred compounds of formula (I) are those wherein wherein the amino acid-residue of R4 is a glycine-residue or an alanine-residue.

Specific highly preferred neutrophil elastase inhibitors having an R4 is a glycine-residue are as follows:

N-[o-(p-pivaloyloxybenzene)sulfonylaminobenzoyl]glycine,

N-[2-(p-pivaloyloxybenzene)sulfonylamino-5-chlorobenzoyl]glycine,

N-[5-methylthio-2-(p-pivaloyloxybenzene)sulfonylaminobenzoyl]glycine,

N-[2-(p-pivaloyloxybenzene)sulfonylamino-5-propylthiobenzoyl]glycine,

N-[5-methyl-2-(p-pivaloyloxybenzene)sulfonylaminobenzoyl]glycine, and

N-[o-(p-pivaloyloxybenzene)sulfonylaminobenzoyl]glycine methylester.

Specific highly preferred neutrophil elastase inhibitors having an R4 is a alanine-residue are as follows:

N-[o-(3-methyl-4-pivaloyloxybenzene)sulfonylaminobenzoyl]-d 1-alanine,

N-[o-(3-methyl-4-pivaloyloxybenzene)sulfonylaminobenzoyl]-beta-alanine,

N-[o-(e-methyl-4-pivaloyloxybenzene)sulfonylaminobenzoyl]-1-alanine,

N-[5-chloro-2-(3-methyl-4-pivaloyloxybenzene)sulfonylaminobenzoyl]-1-alanine and

N-[5-chloro-2-(3-methyl-4-pivaloyloxybenzene)sulfonylamino-benzoyl]-beta -alanine.

Most preferred is the compound represented by the structural formula (II):

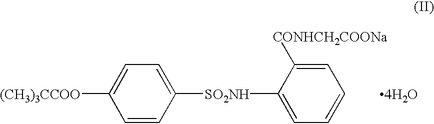

As acid addition salts of the compound of the general formula (I) are preferred non-toxic and water-soluble salts.

Suitable acid addition salts include, for example, an inorganic acid addition salt such as hydrochloride, hydrobromide, hydroiodide, sulfate, phosphate, nitrate, or an organic acid addition salt such as acetate, lactate, tartrate, benzoate, citrate, methanesulfonate, ethanesulfonate, benzenesulfonate, toluenesulfonate, isethionate, glucuronate, gluconate.

The compounds of the present invention of the general formula (I) may be converted into the corresponding salts by known methods. Non-toxic and water-soluble salts are preferable. Suitable salts, for example, are as follows: salts of alkaline metal (sodium, potassium etc.), salts of alkaline earth metal (calcium, magnesium etc.), ammonium salts, salts of pharmaceutically acceptable organic amine (tetramethylammonium, triethylamine, methylamine, dimethylamine, cyclopentylamine, benzylamine, phenethylamine, piperidineamine, monoethanolamine, diethanolamine, tris (hydroxymethyl)amine, lysine, arginine, N-methyl-D-glucamine etc.).

Certain compounds used as either neutrophil elastase inhibitors or sPLA2 inhibitors in the composition or method of the invention may possess one or more chiral centers and may thus exist in optically active forms. Likewise, when the compounds contain an alkenyl or alkenylene group there exists the possibility of cis- and trans-isomeric forms of the compounds. The R- and S-isomers and mixtures thereof, including racemic mixtures as well as mixtures of cis- and trans-isomers, are contemplated by this invention. Additional asymmetric carbon atoms can be present in a substituent group such as an alkyl group. All such isomers as well as the mixtures thereof are intended to be included in the invention. If a particular stereoisomer is desired, it can be prepared by methods well known in the art by using stereospecific reactions with starting materials which contain the asymmetric centers and are already resolved or, alternatively by methods which lead to mixtures of the stereoisomers and subsequent resolution by known methods. For example, a racemic mixture may be reacted with a single enantiomer of some other compound. This changes the racemic form into a mixture of diastereomers and diastereomers, because they have different melting points, different boiling points, and different solubilities can be separated by conventional means, such as crystallization.

Prodrugs are derivatives of the compounds of the invention which have chemically or metabolically cleavable groups and become by solvolysis or under physiological conditions the compounds of the invention which are pharmaceutically active in vivo. Derivatives of the compounds of this invention have activity in both their acid and base derivative forms, but the acid derivative form often offers advantages of solubility, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Particularly preferred esters as prodrugs are methyl, ethyl, propyl, isopropyl, n-butyl, isobutyl, tert-butyl, morpholinoethyl, and N,N-diethylglycolamido.

N,N-diethylglycolamido ester prodrugs may be prepared by reaction of the sodium salt of a compound of Formula (I) (in a medium such as dimethylformamide) with 2-chloro-N,N-diethylacetamide (available from Aldrich Chemical Co., Milwaukee, Wis. USA; Item No. 25,099-6).

Morpholinylethyl ester prodrugs may be prepared by reaction of the sodium salt of a compound of Formula (I) (in a medium such as dimethylformamide) 4-(2-chloroethyl)morpholine hydrochloride (available from Aldrich Chemical Co., Milwaukee, Wis. USA, Item No. C4,220-3)

III. Preparation of the sPLA₂ Inhibitor Ingredient of the Invention.

All types of sPLA₂ inhibitors are generally useful in the practice in this invention.

Exemplary of classes of suitable sPLA₂ useful in the the method of the invention for treatment of sepsis are the following:

1H-indole-3-glyoxylamides

1H-indole-3-hydrazides

1H-indole-3-acetamides

1H-indole-1-glyoxylamides

1H-indole-1-hydrazides

1H-indole-1-acetamides

indolizine-1-acetamides

indolizine-1-acetic acid hydrazides

indolizine-1-glyoxylamides

indene-1-acetamides

indene-1-acetic acid hydrazides

indene-1-glyoxylamides

carbazoles & tetrahydrocarbazoles

pyrazoles

phenyl glyoxamides

pyrroles

naphthyl glyoxamides

phenyl acetamides

naphthyl acetamides

Each of the above sPLA₂ inhibitor types is discussed in the following sections (a) through (m) wherein details of their molecular configuration are given along with methods for their preparation.

a) The 1H-indole-3-glyoxylamide sPLA₂ inhibitors and method of making them are described in U.S. Pat. No. 5,654,326, the entire disclosure of which is incorporated herein by reference. Another method of making 1H-indole-3-glyoxylamide sPLA₂ inhibitors is described in U.S. patent application Ser. No. 09/105,381, filed Jun. 26, 1998 and titled, “Process for Preparing 4-substituted 1-H-Indole-3-glyoxyamides” the entire disclosure of which is incorporated herein by reference.

U.S. patent application Ser. No. 09/105,381 discloses the following process having steps (a) thru (i):

Preparing a compound of the formula (I) or a pharmaceutically acceptable salt or prodrug derivative thereof

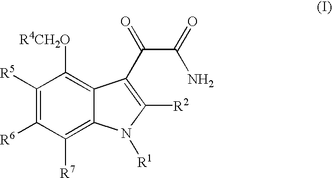

wherein:

R¹ is selected from the group consisting of -C₇-C₂₀ alkyl,

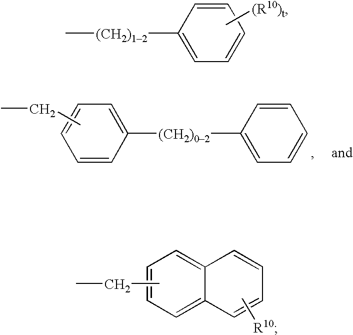

where

R¹⁰ is selected from the group consisting of halo, C₁-C₁₀ alkyl, C₁-C₁₀ alkoxy, —S—(C₁-C₁₀ alkyl) and halo(C₁-C₁₀)alkyl, and t is an integer from 0 to 5 both inclusive;

R² is selected from the group consisting of hydrogen, halo, C₁-C₃ alkyl, C₃-C₄ cycloalkyl, C₃-C₄ cycloalkenyl, —O—(C₁-C₂ alkyl), —S—(C₁-C₂ alkyl), aryl, aryloxy and HET;

R⁴ is selected from the group consisting of —CO₂H, —SO₃H and —P(O)(OH)₂ or salt and prodrug derivatives thereof; and

R⁵, R⁶ and R⁷ are each independently selected from the group consisting of hydrogen, (C₁-C₆)alkyl, (C₁-C₆)alkoxy, halo(C₁-C₆)alkoxy, halo(C₂-C₆)alkyl, bromo, chloro, fluoro, iodo and aryl;

which process comprises the steps of:

a) halogenating a compound of formula X

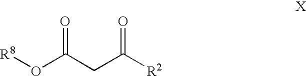

where R⁸ is (C₁-C₆)alkyl, aryl or HET; with SO₂Cl₂ to form a compound of formula IX

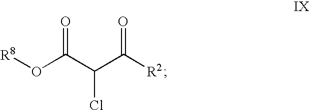

b) hydrolyzing and decarboxylating a compound of formula IX

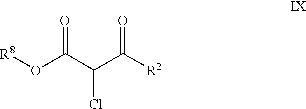

to form a compound of formula VIII

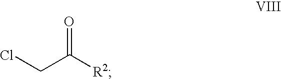

c) alkylating a compound of formula VII

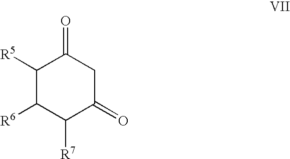

with a compound of formula VIII

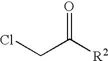

to form a compound of formula VI

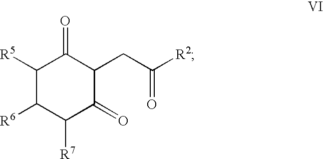

d) aminating and dehydrating a compound of formula VI

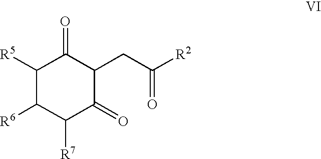

with an amine of the formula R¹NH₂ in the presence of a solvent that forms and azeotrope with water to form a compound of formula V;

e) oxidizing a compound of formula V

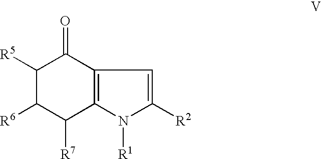

by refluxing in a polar hydrocarbon solvent having a boiling point of at least 150° C. and a dielectric constant of at least 10 in the presence of a catalyst to form a compound of formula IV

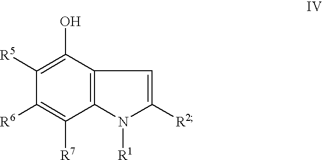

f) alkylating a compound of the formula IV

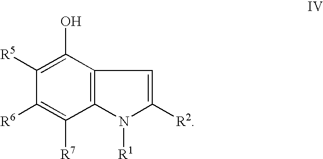

with an alkylating agent of the formula XCH₂R^{4a} where X is a leaving group and R^{4a} is —CO₂R^{4b}, —SO₃R^{4b}, —P(O)(OR^{4b})₂ or —P(O)(OR^{4b})H, where R^{4b} is an acid protecting group to form a compound of formula III

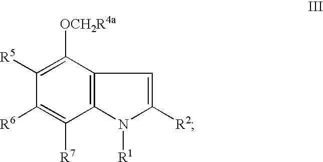

g) reacting a compound of formula III

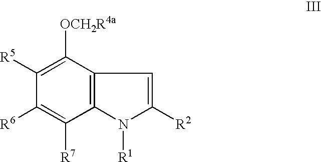

with oxalyl chloride and ammonia to form a compound of formula II

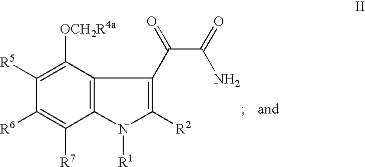

h) optionally hydrolyzing a compound of formula II

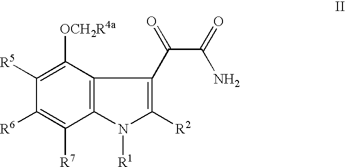

to form a compound of formula I; and

i) optionally salifying a compound of formula I.

The synthesis methodology for making the 1H-indole-3-glyoxylamide sPLA₂ inhibitor may be by any suitable means available to one skilled in the chemical arts. However, such methodology is not part of the present invention which is a method of use, specifically, a method of treating mammal afflicted or susceptible to sepsis.

The method of the invention is for treatment of a mammal, including a human, afflicted sepsis, said method comprising administering to said human a therapeutically effective amount of the compound represented by formula (Ia), or a pharmaceutically acceptable salt or prodrug derivative thereof;

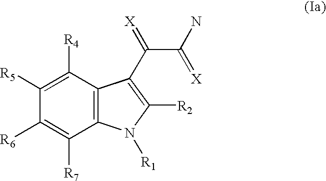

wherein

both X are oxygen;

R₁ is selected from the group consisting of

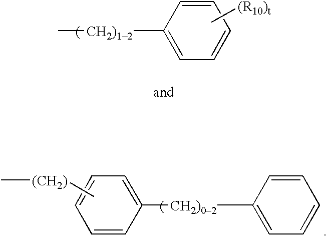

where R₁₀ is a radical independently selected from halo, C₁-C₁₀ alkyl, C₁-C₁₀ alkoxy, —S—(C₁-C₁₀ alkyl), and C₁-C₁₀ haloalkyl and t is a number from 0 to 5;

R₂ is selected from the group; halo, cyclopropyl, methyl, ethyl, and propyl;

R₄ and R₅ are independently selected from hydrogen, a non-interfering substituent, or the group, -(Lₐ)-(acidic group); wherein -(Lₐ)- is an acid linker; provided, the acid linker group, -(Lₐ)-, for R₄ is selected from the group consisting of;

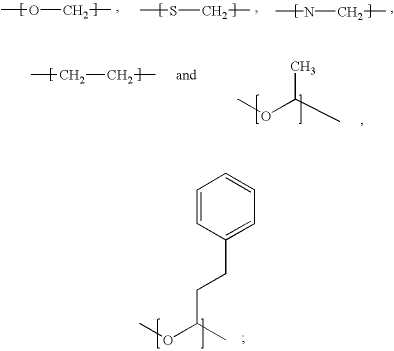

and provided, the acid linker, -(Lₐ)- for R₅ is selected from group consisting of;

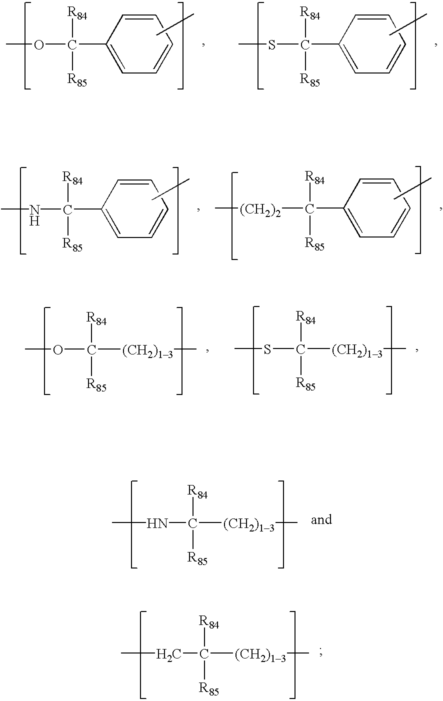

wherein R₈₄ and R₈₅ are each independently selected from hydrogen, C₁-C₁₀ alkyl, aryl, C₁-C₁₀ alkaryl, C₁-C₁₀ aralkyl, carboxy, carbalkoxy, and halo; and provided, that at least one of R₄ and R₅ must be the group, -(Lₐ)-(acidic group) and wherein the (acidic group) on the group -(Lₐ)-(acidic group) of R₄ or R₅ is selected from —CO₂H, —SO₃H, or —P(O)(OH)₂;

R₆ and R₇ are each independently selected form hydrogen and non-interfering substituents, with the non-interfering substituents being selected from the group consisting of the following: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8.

Preferred for practicing the method of the invention and preparing compositions of the invention are 1H-indole-3-glyoxylamide compounds and all corresponding pharmaceutically acceptable salts, solvates and prodrug derivatives thereof which are useful in the method of the invention include the following:

(A) [[3-(2-Amino-1,2-dioxoethyl)-2-methyl-1-(phenylmethyl)-1H-indol-4-yl]oxy]acetic acid,

(B) dl-2-[[3-(2-Amino-1,2-dioxoethyl)-2-methyl-1-(phenylmethyl)-1H-indol-4-yl]oxy]propanoic acid,

(C) [[3-(2-Amino-1,2-dioxoethyl)-1-([1,1′-biphenyl]-2-ylmethyl)-2-methyl-1H-indol-4-yl]oxy]acetic acid,

(D) [[3-(2-Amino-1,2-dioxoethyl)-1-([1,1′-biphenyl]-3-ylmethyl)-2-methyl-1H-indol-4-yl]oxy]acetic acid,

(E) [[3-(2-Amino-1,2-dioxoethyl)-1-([1,1′-biphenyl]-4-ylmethyl)-2-methyl-1H-indol-4-yl]oxy]acetic acid,

(F) [[3-(2-Amino-1,2-dioxoethyl)-1-[(2,6-dichlorophenyl)methyl]-2-methyl-1H-indol-4-yl]oxy]acetic acid

(G) [[3-(2-Amino-1,2-dioxoethyl)-1-[4(-fluorophenyl)methyl]-2-methyl-1H-indol-4-yl]oxy]acetic acid,

(H) [[3-(2-Amino-1,2-dioxoethyl)-2-methyl-1-[(1-naphthalenyl)methyl]-1H-indol-4-yl]oxy]acetic acid,

(I) [[3-(2-Amino-1,2-dioxoethyl)-2-ethyl-1-(phenylmethyl)-1H-indol-4-yl]oxy]acetic acid,

(J) [[3-(2-Amino-1,2-dioxoethyl)-1-[(3-chlorophenyl)methyl]-2-ethyl-1H-indol-4-yl]oxy]acetic acid,

(K) [[3-(2-Amino-1,2-dioxoethyl)-1-([1,1′-biphenyl]-2-ylmethyl)-2-ethyl-1H-indol-4-yl]oxy]acetic acid,

(L) [[3-(2-amino-1,2-dioxoethyl)-1-([1,1′-biphenyl]-2-ylmethyl)-2-propyl-1H-indol-4-yl]oxy]acetic acid,

(M) [[3-(2-Amino-1,2-dioxoethyl)-2-cyclopropyl-1-(phenylmethyl)-1H-indol-4-yl]oxy]acetic acid,

(N) [[3-(2-Amino-1,2-dioxoethyl)-1-([1,1′-biphenyl]-2-ylmethyl)-2-cyclopropyl-1H-indol-4-yl]oxy]acetic acid,

(O) 4-[[3-(2-Amino-1,2-dioxoethyl)-2-ethyl-1-(phenylmethyl)-1H-indol-5-yl]oxy]butanoic acid,

(P) mixtures of (A) through (P) in any combination.

Particularly useful as sPLA₂ inhibitors are prodrugs of the compounds of formula (I) and named compounds (A) thru (O). The preferred prodrugs are the aromatic and aliphatic esters, such as the methyl ester, ethyl ester, n-propyl ester, isopropyl ester, n-butyl ester, sec-butyl, tert-butyl ester, N,N-diethylglycolamido ester, and morpholino-N-ethyl ester. Methods of making ester prodrugs are disclosed in U.S. Pat. No. 5,654,326. Additional methods of prodrug synthesis are disclosed in U.S. Provisional Patent Application Serial No. 60/063,280 filed Oct. 27, 1997 (titled, N,N-diethylglycolamido ester Prodrugs of Indole sPLA₂ Inhibitors), the entire disclosure of which is incorporated herein by reference; U.S. Provisional Patent Application Serial No. 60/063,646 filed Oct. 27, 1997 (titled, Morpholino-N-ethyl Ester Prodrugs of Indole sPLA₂ Inhibitors), the entire disclosure of which is incorporated herein by reference; and U.S. Provisional Patent Application Serial No. 60/063,284 filed Oct. 27, 1997 (titled, Isopropyl Ester Prodrugs of Indole sPLA₂ Inhibitors), the entire disclosure of which is incorporated herein by reference.

Most preferred in the practice of the method of the invention are the acid, sodium salt, methyl ester, and morpholino-N-ethyl ester forms of [[3-(2-Amino-1,2-dioxoethyl)-2-ethyl-1-(phenylmethyl)-1H-indol-4-yl]oxy]acetic acid as represented by the following formulae:

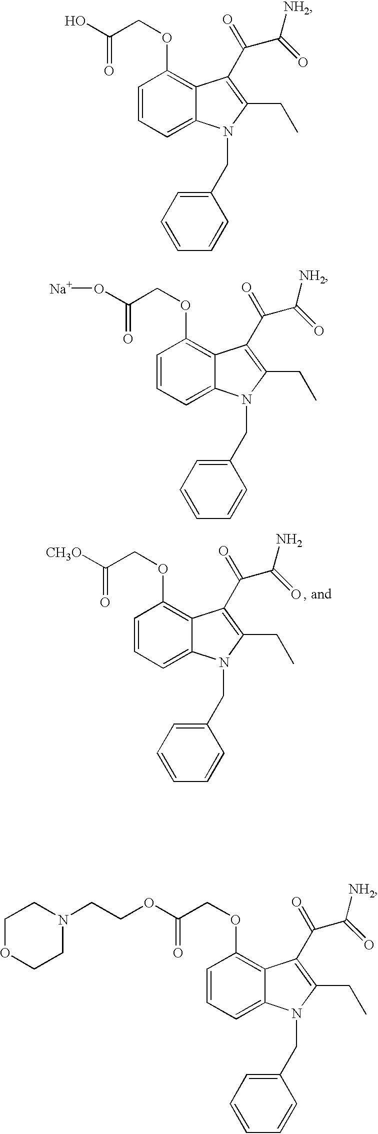

Another highly preferred compound is the indole-3-glyoxylamide morpholino ethyl ester of represented by the formula:

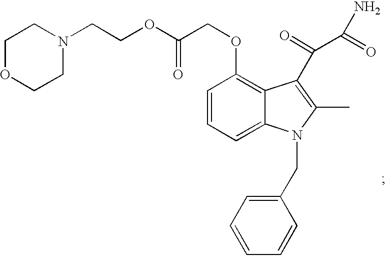

the preparation of which is further described in U.S. provisional patent application S No. 60/063,646 filed Oct. 27, 1997.

Synthesis methods for 1H-indole-3-glyoxylamide sPLA₂ inhibitors are additionally depicted in the following reaction scheme:

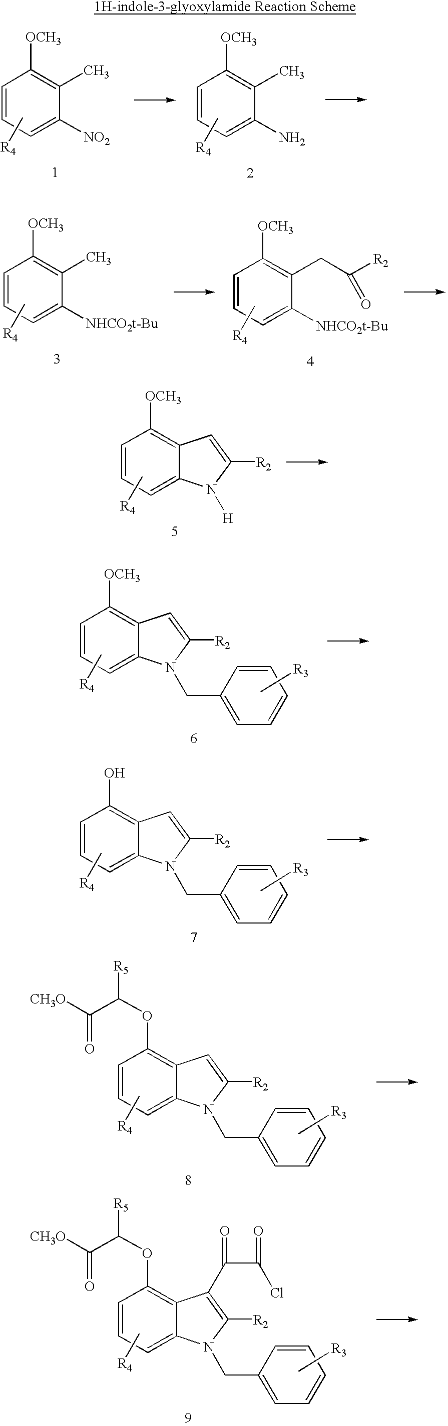

Explanation of Reaction Scheme:

To obtain the glyoxylamides substituted in the 4-position with an acidic function through an oxygen atom, the reactions outlined in scheme 1 are used (for conversions 1 through 5, see ref. Robin D. Clark, Joseph M. Muchowski, Lawrence E. Fisher, Lee A. Flippin, David B. Repke, Michel Souchet, Synthesis, 1991, 871-878, the disclosures of which are incorporated herein by reference) The ortho-nitrotoluene, 1, is readily reduced to the 2-methylaniline, 2, using Pd/C as catalyst. The reduction can be carried out in ethanol or tetrahydrofuran (THF) or a combination of both, using a low pressure of hydrogen. The aniline, 2, on heating with di-tert-butyl dicarbonate in THF at reflux temperature is converted to the N-tert-butylcarbonyl derivative, 3, in good yield. The dilithium salt of the dianion of 3 is generated at −40 to −20° C. in THF using sec-butyl lithium and reacted with the appropriately substituted N-methoxy-N-methylalkanamide. This product, 4, may be purified by crystallization from hexane, or reacted directly with trifluoroacetic acid in methylene chloride to give the 1,3-unsubstituted indole 5. The 1,3-unsubstituted indole 5 is reacted with sodium hydride in dimethylformamide at room temperature (20-25° C.) for 0.5-1.0 hour. The resulting sodium salt of 5 is treated with an equivalent of arylmethyl halide and the mixture stirred at a temperature range of 0-100° C., usually at ambient room temperature, for a period of 4 to 36 hours to give the 1-arylmethylindole, 6. This indole, 6, is O-demethylated by stirring with boron tribromide in methylene chloride for approximately 5 hours (see ref. Tsung-Ying Shem and Charles A Winter, Adv. Drug Res., 1977, 12, 176, the disclosure of which is incorporated herein by reference). The 4-hydroxyindole, 7, is alkylated with an alpha bromoalkanoic acid ester in dimethylformamide (DMF) using sodium hydride as a base, with reactions conditions similar to that described for the conversion of 5 to 6. The a-[(indol-4-yl)oxy]alkanoic acid ester, 8, is reacted with oxalyl chloride in methylene chloride to give 9, which is not purified but reacted directly with ammonia to give the glyoxamide 10. This product is hydrolyzed using 1N sodium hydroxide in MeOH. The final glyoxylamide, 11, is isolated either as the free carboxylic acid or as its sodium salt or in both forms.

The most preferred compound, [[3-(2-Amino-1,2-dioxoethyl)-2-ethyl-1-(phenylmethyl)-1H-indol-4-yl]oxy]acetic acid (as well as its sodium salt and methyl ester) useful in the practice of the method of the invention, may be prepared by the following procedure:

Preparation of [[3-(2-Amino-1,2-dioxoethyl)-2-ethyl-1-(phenylmethyl)-1H-indol-4-yl]oxy]acetic acid, a compound represented by the formula:

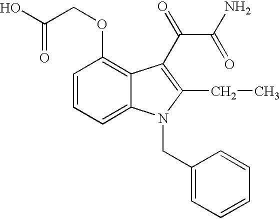

Part A. Preparation of 2-Ethyl-4-methoxy-1H-indole.

A solution of 140 mL (0.18 mol) of 1.3M sec-butyl lithium in cyclohexane was added slowly to N-tert-butoxycarbonyl-3-methoxy-2-methylaniline (21.3 g, 0.09 mol) in 250 mL of THF keeping the temperature below −40° C. with a dry ice-ethanol bath. The bath was removed and the temperature allowed to rise to 0° C. and then the bath replaced. After the temperature had cooled to −60° C., 18.5 g (0.18 mol) of N-methoxy-N-methylpropanamide in an equal volume of THF was added dropwise. The reaction mixture was stirred 5 minutes, the cooling bath removed and stirred an additional 18 hours. It was then poured into a mixture of 300 mL of ether and 400 mL of 0.5N HCl. The organic layer was separated, washed with water, brine, dried over MgSO₄, and concentrated at reduced pressure to give 25.5 g of a crude of 1-[2-(tert-butoxycarbonylamino)-6-methoxyphenyl]-2-butanone. This material was dissolved in 250 mL of methylene chloride and 50 mL of trifluoroacetic acid and stirred for a total of 17 hours. The mixture was concentrated at reduced pressure and ethyl acetate and water added to the remaining oil. The ethyl acetate was separated, washed with brine, dried (MgSO₄) and concentrated. The residue was chromatographed three times on silica eluting with 20% EtOAc/hexane to give 13.9 g of 2-ethyl-4-methoxy-1H-indole.

Analyses for C₁₁H₁₃NO:

| | | | |
|---|---|---|---|
| Calculated: | C, 75.40; | H, 7.48; | N, 7.99; |
| Found: | C, 74.41; | H, 7.64; | N, 7.97. |

Part B. Preparation of 2-Ethyl-4-methoxy-1-(phenylmethyl)-1H-indole.

2-Ethyl-4-methoxy-1H-indole (4.2 g, 24 mmol) was dissolved in 30 mL of DMF and 960 mg (24 mmol) of 60% NaH/minerial oil was added. After 1.5 hours, 2.9 mL(24 mmol) of benzyl bromide was added. After 4 hours, the mixure was diluted with water and extracted twice with ethyl acetate. The combined ethyl acetate was washed with brine, dried (MgSO₄) and concentrated at reduced pressure. The residue was chromatographed on silica gel and eluted with 20% EtOAc/hexane to give 3.1 g (49% yield) of 2-ethyl-4-methoxy-1-(phenylmethyl)-1H-indole.

Part C. Preparation of 2-Ethyl-4-hydroxy-1-(phenylmethyl)-1H-indole.

3.1 g (11.7 mmol) of 2-ethyl-4-methoxy-1-(phenylmethyl)-1H-indole was O-demethylated by treating it with 48.6 mL of 1M BBr₃ in methylene chloride with stirring at room temperature for 5 hours, followed by concentration at reduced pressure. The residue was dissolved in ethyl acetate, washed with brine and dried (MgSO₄). After concentrating at reduced pressure, the residue was chromatographed on silica gel eluting with 20% EtOAc/hexane to give 1.58 g (54% yield) of 2-ethyl-4-hydroxy-1-(phenylmethyl)-1H-indole, mp, 86-90° C.

Analyses for C₁₇H₁₇NO:

| | | | |
|---|---|---|---|
| Calculated: | C, 81.24; | H, 6.82; | N, 5.57; |
| Found: | C, 81.08; | H, 6.92; | N, 5.41. |

Part D. Preparation of [[2-Ethyl-1-(phenylmethyl)-1H-indol-4-yl]oxy]acetic Acid Methyl Ester.

2-ethyl-4-hydroxy-1-(phenylmethyl)-1H-indole (1.56 g, 6.2 mmol) was added to a mixture of 248 mg (6.2 mmol) of 60% NaH/mineral oil in 20 mL DMF and stirred for 0.67 hour.

Then 0.6 mL(6.2 mmol) of methyl bromoacetate was added and stirring was continued for 17 hours. The mixture was diluted with water and extracted with ethyl acetate. The ethyl acetate solution was washed with brine, dried (MgSO₄), and concentrated at reduced pressure. The residue was chromatographed on silica gel eluting with 20% EtOAc/hexane, to give 1.37 g (69% yield) of [[2-ethyl-1-(phenylmethyl)-1H-indol-4-yl]oxy]acetic acid methyl ester, 89-92° C.

Analyses for C₂₀H₂₁NO₃:

| | | | |
|---|---|---|---|
| Calculated: | C, 74.28; | H, 6.55; | N, 4.33; |
| Found: | C, 74.03; | H, 6.49; | N, 4.60. |

Part E. Preparation of [[3-(2-Amino-1,2-dioxoethyl)-2-ethyl-1-(phenylmethyl)-1H-indol-4-yl]oxy]acetic Acid Methyl Ester.

Oxalyl chloride (0.4 mL, 4.2 mmol) was added to 1.36 g (4.2 mmol) of [[2-ethyl-1-(phenylmethyl)-1H-indol-4-yl]oxy]acetic acid methyl ester in 10 mL of methylene chloride and the mixture stirred for 1.5 hours. The mixture was concentrated at reduced pressure and residue taken up in 10 mL of methylene chloride. Anhydrous ammonia was bubbled in for 0.25 hours, the mixture stirred for 1.5 hours and evaporated at reduced pressure. The residue was stirred with 20 mL of ethyl acetate and the mixture filtered. The filtrate was concentrated to give 1.37 g of a mixture of [[3-(2-amino-1,2-dioxoethyl)-2-ethyl-1-(phenylmethyl)-1H-indol-4-yl]oxy]acetic acid methyl ester and ammonium chloride. This mixture melted at 172-187° C.

Part F. Preparation of [[3-(2-Amino-1,2-dioxoethyl)-2-ethyl-1-(phenylmethyl)-1H-indol-4-yl]oxy]acetic Acid.

A mixture of 788 mg (2 mmol) of [3-(2-amino-1,2-dioxoethyl)-2-ethyl-1-(phenylmethyl)-1H-indol-4-yl]oxy]1-acetic acid methyl ester, 10 mL of In NaOH and 30 mL of MeOH is heated to maintain reflux for 0.5 hour, stirred at room temperature for 0.5 hour and concentrated at reduced pressure. The residue is taken up in ethyl acetate and water, the aqueous layer separated and made acidic to pH 2-3 with 1N HCl. The precipitate is filtered and washed with ethyl acetate to give 559 mg (74% yield) of [[3-(2-amino-1,2-dioxoethyl)-2-ethyl-1-(phenylmethyl)-1H-indol-4-yl]oxy]acetic acid, mp, 230-234° C.

Analyses for C₂₁H₂₀N₂O₅:

| | | | |
|---|---|---|---|
| Calculated: | C, 65.96; | H, 5.80; | N, 7.33; |
| Found: | C, 66.95; | H, 5.55; | N, 6.99. |

b) 1H-indole-3-hydrazide sPLA₂ inhibitors useful in practicing the method of the invention are described in U.S. Pat. No. 5,578,634; the entire disclosure of which is incorporated herein by reference. The method of the invention is for treatment of a mammal, including a human, afflicted with sepsis, said method comprising administering to said human a therapeutically effective amount of the described as 1H-indole-3-acetic acid hydrazides represented by the formula (Ib), and pharmaceutically acceptable salts, and prodrugs thereof;

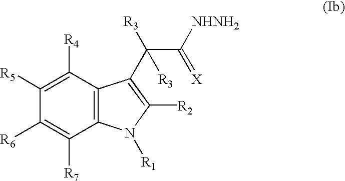

wherein;

X is oxygen or sulfur;

R₁ is selected from groups (i), (ii) and (iii) where;

(i) is C₄-C₂₀ alkyl, C₄-C₂₀ alkenyl, C₄-C₂₀ alkynyl, C₄-C₂₀ haloalkyl, C₄-C₁₂ cycloalkyl, or

(ii) is aryl or aryl substituted by halo, —CN, —CHO, —OH, —SH, C₁-C₁₀ alkylthio, C₁-C₁₀ alkoxy, C₁-C₁₀ alkyl, carboxyl, amino, or hydroxyamino;

(iii) is

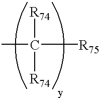

where y is from 1 to 8, R₇₄ is, independently, hydrogen or C₁-C₁₀ alkyl, and R₇₅ is aryl or aryl substituted by halo, —CN, —CHO, —OH, nitro, phenyl, —SH, C₁-C₁₀ alkylthio, C₁-C₁₀ alkoxy, C₁-C₁₀ alkyl, amino, hydroxyamino or a substituted or unsubstituted 5- to 8-membered heterocyclic ring;

R₂ is halo, C₁-C₃ alkyl, ethenyl, C₁-C₂ alkylthio, C₁-C₂ alkoxy, —CHO, —CN;

each R₃ is independently hydrogen, C₁-C₃ alkyl, or halo;

R₄, R₅, R₆, and R₇ are each independently hydrogen, C₁-C₁₀ alkyl, C₁-C₁₀ alkenyl, C₁-C₁₀ alkynyl, C₃-C₈ cycloalkyl, aryl, aralkyl, or any two adjacent hydrocarbyl groups in the set R₄, R₅, R₆, and R₇ combined with the ring carbon atoms to which they are attached to form a 5- or 6-membered substituted or unsubstituted carbocyclic ring; or C₁-C₁₀ haloalkyl, C₁-C₁₀ alkoxy, C₁-C₁₀ haloalkoxy, C₄-C₈ cycloalkoxy, phenoxy, halo, hydroxy, carboxyl, —SH, —CN, —S(C₁-C₁₀ alkyl), arylthio, thioacetal, —C(O)O(C₁-C₁₀ alkyl), hydrazino, hydrazido, —NH₂, —NO₂, —NR₈₂R₈₃, and —C(O)NR₈₂R₈₃, where, R₈₂ and R₈₃ are independently hydrogen, C₁-C₁₀ alkyl, C₁-C₁₀ hydroxyalkyl, or taken together with N, R₈₂ and R₈₃ form a 5- to 8-membered heterocyclic ring; or a group having the formula;

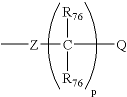

where,

each R₇₆ is independently selected from hydrogen, C₁-C₁₀ alkyl, hydroxy, or both R₇₆ taken together are ═O;

p is 1 to 8,

Z is a bond, —O—, —N(C₁-C₁₀ alkyl)—, —NH, or —S—; and

Q is —CON(R₈₂R₈₃), -5-tetrazolyl, —SO₃H,

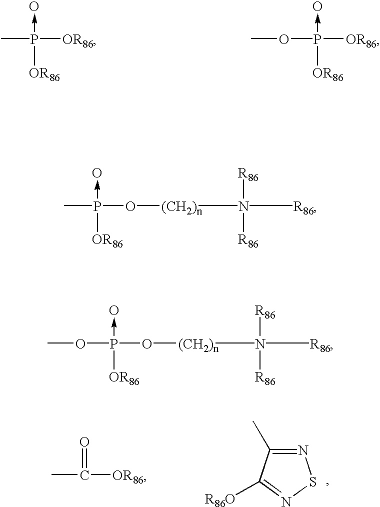

where R₈₆ is independently selected from hydrogen, a metal, or C₁-C₁₀ alkyl.

The synthesis of the 1H-indole-3-acetic acid hydrazides of structure (I) can be accomplished by known methods such as outlined in the following reaction schemes:

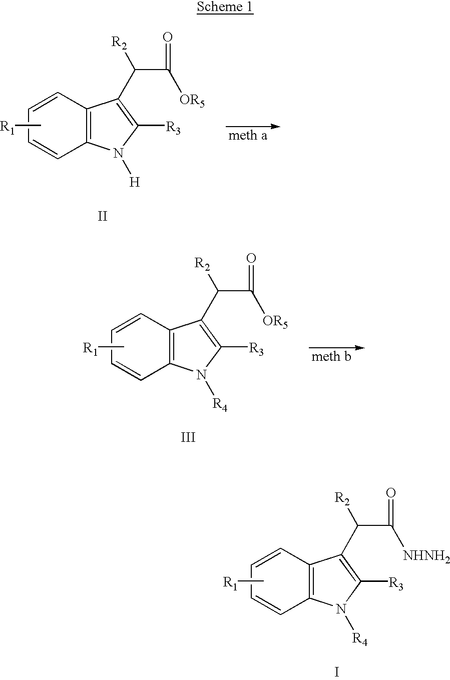

The 1H-indole-3-acetic acid ester can be readily alkylated by an alkyl halide or arylalkyl halide in a solvent such as N,N-dimethylformamide(DMF) in the presence of a base(meth a) to give the intermediate 1-alkyl-1H-indole-3-acetic acid esters, III. Bases such as potassium t-butoxide and sodium hydride were particularity useful. It is advantageous to react the indole, II, with the base to first form the salt of II and then add the alkylating agent. Most alkylations can be carried out at room temperature. Treatment of the 1-alkyl-1H-indole-3-acetic acid esters, III, with hydrazine or hydrazine hydrate in ethanol(meth b) gives the desired 1-alkyl-1H-indole-3-acetic acid hydrazides, I. This condensation to form I is usually carried out at the reflux temperature of the solvent for a period of 1 to 24 hours.

c) 1H-indole-3-acetamide sPLA₂ inhibitors and methods of making these inhibitors are set out in U.S. Pat. No. 5,684,034, the entire disclosure of which is incorporated herein by reference. These inhibitors are useful ingredients in the compositons of the invention and the method of the invention for treatment of a mammal, including a human, afflicted with sepsis.

Useful inhibitors are represented by formula (IIb), and pharmaceutically acceptable salts and prodrug derivatives thereof,

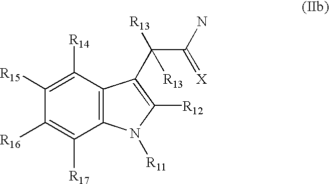

wherein;

X is oxygen or sulfur;

R₁₁ is selected from groups (i), (ii) (iii) and (iv) where;

(i) is C₆-C₂₀ alkyl, C₆-C₂₀ alkenyl, C₆-C₂₀ alkynyl, C₆-C₂₀ haloalkyl, C₄-C₁₂ cycloalkyl, or

(ii) is aryl or aryl substituted by halo, nitro, —CN, —CHO, —OH, —SH, C₁-C₁₀ alkyl, C₁-C₁₀ alkylthio, C₁-C₁₀ alkoxyl, carboxyl, amino, or hydroxyamino; or

(iii) is —(CH₂)ₙ—(R₈₀), or —(NH)—(R₈₁), where n is 1 to 8, and R₈₀ is a group recited in (i), and R₈₁ is selected from a group recited in (i) or (ii);

(iv) is

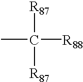

where R₈₇ is hydrogen or C₁-C₁₀ alkyl, and R₈₈ is selected from the group; phenyl, naphthyl, indenyl, and biphenyl, unsubstituted or substituted by halo, —CN, —CHO, —OH, —SH, C₁-C₁₀ alkylthio, C₁-C₁₀ alkoxyl, phenyl, nitro, C₁-C₁₀ alkyl, C₁-C₁₀ haloalkyl, carboxyl, amino, hydroxyamino; or a substituted or unsubstituted 5 to 8 membered heterocyclic ring;

R₁₂ is halo, C₁-C₂ alkylthio, or C₁-C₂ alkoxy;

each R₁₃ is independently hydrogen, halo, or methyl;

R₁₄, R₁₅, R₁₆, and R₁₇ are each independently hydrogen, C₁-C₁₀ alkyl, C₁-C₁₀ alkenyl, C₁-C₁₀ alkynyl, C₃-C₈ cycloalkyl, aryl, aralkyl, or any two adjacent hydrocarbyl groups in the set R₁₄, R₁₅, R₁₆, and R₁₇, combine with the ring carbon atoms to which they are attached to form a 5 or 6 membered substituted or unsubstituted carbocyclic ring; or C₁-C₁₀ haloalkyl, C₁-C₁₀ alkoxy, C₁-C₁₀ haloalkoxy, C₄-C₈ cycloalkoxy, phenoxy, halo, hydroxy, carboxyl, —SH, —CN, C₁-C₁₀ alkylthio, arylthio, thioacetal, —C(O)O(C₁-C₁₀ alkyl), hydrazide, hydrazino, hydrazido, —NH₂, —NO₂, —NR₈₂R₈₃, and —C(O)NR₈₂R₈₃, where, R₈₂ and R₈₃ are independently hydrogen, C₁-C₁₀ alkyl, C₁-C₁₀ hydroxyalkyl, or taken together with N, R₈₂ and R₈₃ form a 5- to 8-membered heterocyclic ring; or

a group having the formula;

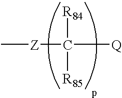

where,

R₈₄ and R₈₅ are each independently selected from hydrogen, C₁-C₁₀ alkyl, hydroxy, or R₈₄ and R₈₅ taken together are ═O;

p is 1 to 5,

Z is a bond, —O—, —N(C₁-C₁₀ alkyl)—, —NH—, or —S—; and

Q is —CON(R₈₂R₈₃), -5-tetrazolyl, —SO₃H,

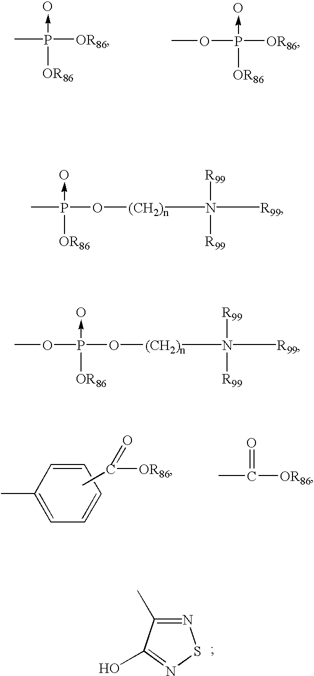

where n is 1 to 8, R₈₆ is independently selected from hydrogen, a metal, or C₁-C₁₀ alkyl, and R₉₉ is selected from hydrogen or C₁-C₁₀ alkyl.

The synthesis of the 1H-indole-3-acetamides of structure (IIb) useful in the method of the invention can be accomplished by known methods. A procedure useful for the syntheses of these compounds is shown in the following reaction scheme:

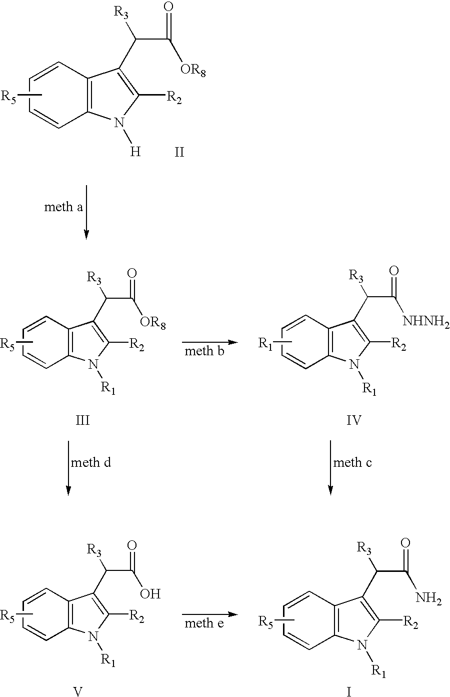

The 1H-indole-3-acetamide II may be alkylated by an alkyl halide or arylalkyl halide in a solvent such as N,N-dimethylformamide (DMF) in the presence of a base (method a) to give intermediate 1-alkyl-1H-indole-3-acetic acid esters, III. Bases such as potassium t-butoxide and sodium hydride are useful. It is advantageous to react the indole, II, with the base to first form the salt of II and then add alkylating agent. Treatment of the 1-alkyl-1H-indole-3-acetic acid esters, III, with hydrazine or hydrazine hydrate in ethanol (method b) gives the desired 1-alkyl-1H-indole-3-acetic acid hydrazides, IV. This condensation to form IV may be carried out at the reflux temperature of the solvent for a period of 1 to 24 hours. The acetic acid hydrazides, IV, are hydrogenated to give the acetamides, I, by heating with Raney nickel in ethanol (method c). The intermediate acetic acid esters, III, can be first hydrolyzed to the acetic acid derivatives, V (method d), which on treatment with an alkyl chloroformate followed by anhydrous ammonia, also give amides, I (method e).

d) 1H-indole-1-functional sPLA₂ inhibitors of the hydrazide, amide, or glyoxylamide types as described in U.S. Pat. No. 5,641,800, the entire disclosure of which is incorporated herein by reference. These inhibitors are useful ingredients in the compositons of the invention and the method of the invention for treatment of a mammal, including a human, afflicted with sepsis.

A 1H-indole-1-acetamide or a pharmaceutically acceptable salt, solvate or prodrug derivative thereof; wherein said compound is represented by the formula (Ic);

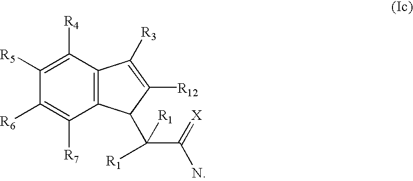

wherein for Formula (Ic);

X is oxygen or sulfur;

each R₁ is independently hydrogen, or C₁-C₃ alkyl;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₆ and R₇ are independently selected from hydrogen, a non-interfering substituent, or the group, -(Lₐ)-(acidic group); wherein -(Lₐ)-, is an acid linker having an acid linker length of 1 to 10; provided, that at least one of R₆ and R₇ must be the group, -(La)-(acidic group);

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

1H-indole-1-hydrazide compounds useful as sPLA₂ inhibitors in the practice of the method and formulation of the compositions of the invention are as follows:

A 1H-indole-1-hydrazide compound or a pharmaceutically acceptable salt, solvate or prodrug derivative thereof; wherein said compound is represented by the formula (IIc);

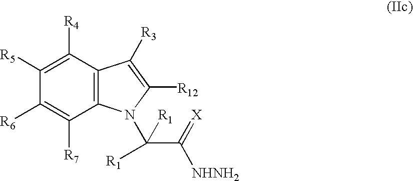

wherein for formula (IIc);

X is oxygen or sulfur;

each R₁ is independently hydrogen, or C₁-C₃ alkyl;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituent; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 † to 3 atoms other than hydrogen;

R₆ and R₇ are independently selected from hydrogen, a non-interfering substituent, or the group, -(Lₐ)-(acidic group); wherein -(Lₐ)-, is an acid linker having an acid linker length of 1 to 10; provided, that at least one of R₆ and R₇ must be the group, -(La)-(acidic group);

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

e) Indolizine sPLA₂ inhibitors and their method of preparation are described in U.S. patent application Ser. No. 08/765,566, filed Jul. 20, 1995 (titled, “Synovial Phospholipase A2 Inhibitor Compounds Having an Indolizine Type Nucleus, Parmaceutical Formulations Containing Said compounds, and Therapeutic Methods of Using said Compounds”), the entire disclosure of which is incorporated herein by reference; and also in European Patent Publication No. 0772596, published May 14, 1997. These inhibitors are useful in the formulation of the compositions of the invention and in the practice of the method of the invention is for treatment of a mammal, including a human, afflicted with sepsis.

Useful 1H-indole-1-functional compounds or pharmaceutically acceptable salts, solvates or prodrug derivatives are represented by the formula (Id);

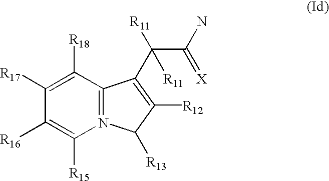

wherein;

X is oxygen or sulfur;

each R₁₁ is independently hydrogen, C₁-C₃ alkyl, or halo;

R₁₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₁₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₁₇ and R₁₈ are independently selected from hydrogen, a non-interfering substituent, or the group, -(Lₐ)-(acidic group); wherein -(Lₐ)-, is an acid linker having an acid linker length of 1 to 10; provided, that at least one of R₁₇ and R₁₈ must be the group, -(Lₐ)-(acidic group); and

R₁₅ and R₁₆ are each independently selected from hydrogen, non-interfering substituent, carbocyclic radical, carbocyclic radical substituted with non-interfering substituents, heterocyclic radical, and heterocyclic radical substituted with non-interfering substituents.

Particularly preferred 1H-indole-1-functional compounds useful as sPLA₂ inhibitors in the practice of the method of the invention are as follows: An indolizine-1-acetic acid hydrazide compound or a pharmaceutically acceptable salt, solvate or prodrug derivative thereof where said compound is represented by the formula (IId);

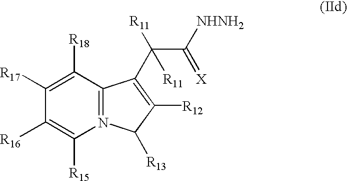

Particularly preferred 1H-indole-1-functional compounds useful as sPLA₂ inhibitors in the practice of the method of the invention are as follows:

An indolizine-1-glyoxylamide compound or a pharmaceutically acceptable salt, solvate or prodrug derivative thereof; wherein said compound is represented by the formula (IIId);

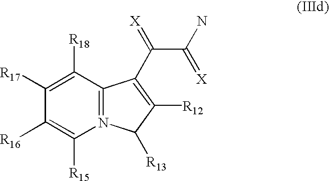

Another preferred 1H-indole-1-functional compounds useful as sPLA₂ inhibitors in the practice of the method of the invention are as follows:

An indolizine-3-acetamide compound or a pharmaceutically acceptable salt, solvate or prodrug derivative thereof; wherein said compound is represented by the formula (IVd), as set out below:

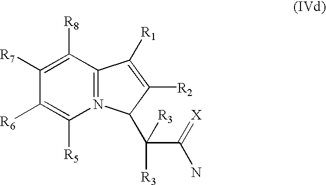

wherein;

X is selected from oxygen or sulfur;

each R₃ is independently hydrogen, C₁-C₃ alkyl, or halo;

R₁ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₅ and R₆ are independently selected from hydrogen, a non-interfering substituent, or the group, -(Lₐ)-(acidic group); wherein -(Lₐ)-, is an acid linker having an acid linker length of 1 to 10; provided, that at least one of R₅ and R₆ must be the group, -(Lₐ)-(acidic group);

R₇ and R₈ are each independently selected from hydrogen, non-interfering substituent, carbocyclic radical, carbocyclic radical substituted with non-interfering substituents, heterocyclic radical, and heterocyclic radical substituted with non-interfering substituents.

Particularly preferred 1H-indole-1-functional compounds useful as sPLA₂ inhibitors in the practice of the method of the invention are as follows: An indolizine-3-hydrazide compound or a pharmaceutically acceptable salt, solvate or prodrug derivative thereof; wherein said compound is represented by the formula (Vd), as set out below:

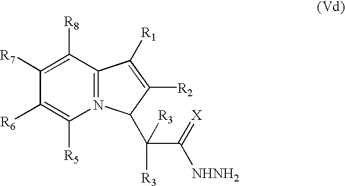

Particularly preferred 1H-indole-1-functional compounds useful as sPLA₂ inhibitors in the practice of the method of the invention are as follows:

An indolizine-3-glyoxylamide compound or a pharmaceutically acceptable salt, solvate or prodrug derivative thereof; wherein said compound is represented by the formula (VId), as set out below:

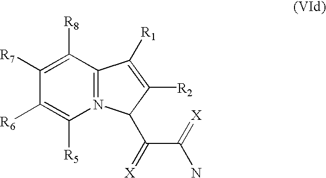

Particularly preferred 1H-indole-1-functional compounds useful as sPLA₂ inhibitors in the practice of the method of the invention are as follows:

An indolizine-1-acetamide functional compound or a pharmaceutically acceptable salt, solvate or prodrug derivative thereof; wherein said compound is selected from the group represented by the following formulae:

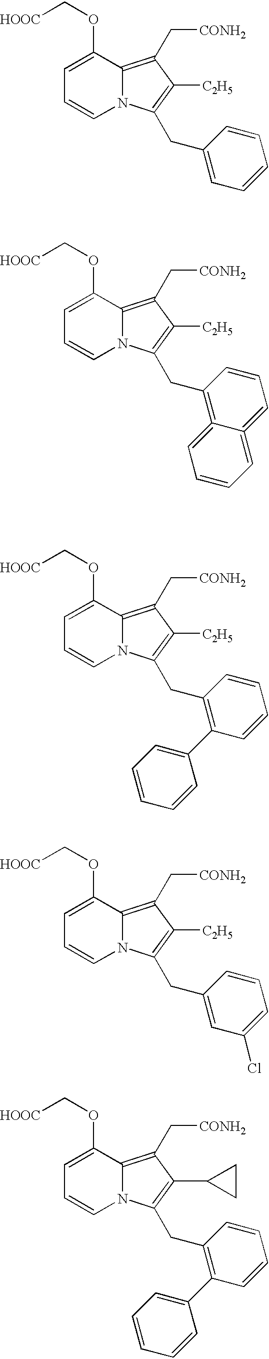

and mixtures of the above compounds.

Other particularly preferred 1H-indole-1-functional compounds useful as sPLA₂ inhibitors in the practice of the method of the invention are as follows:

An indolizine-1-glyoxylamide functional compound and a pharmaceutically acceptable salt, solvate or prodrug derivative thereof; wherein said compound is selected from the group represented by the following formulae:

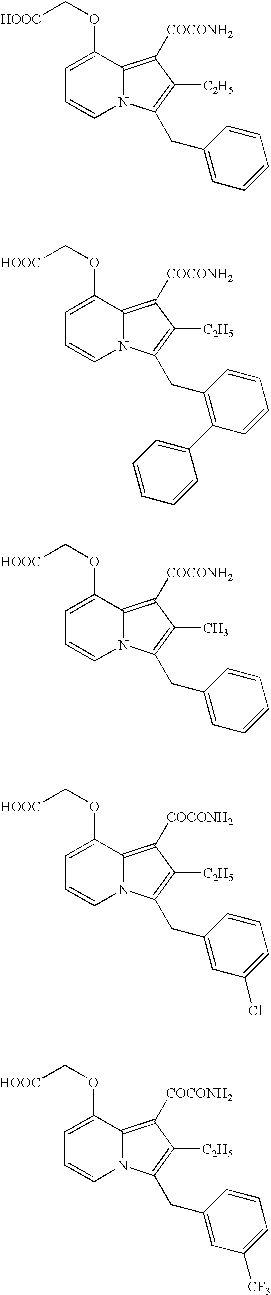

and mixtures of the above compounds.

The indolizine compounds may be made by one of more of the following reaction schemes:

The following abbreviations are used:

| | |
|---|---|
| Bn | benzyl |
| THF | tetrahydrofuran |
| LAH | lithium aluminum hydride |
| LDA | lithium diiopropyl amine |
| DBU | 1,8-diazabicyclo 5.4.0] undec-7-une |

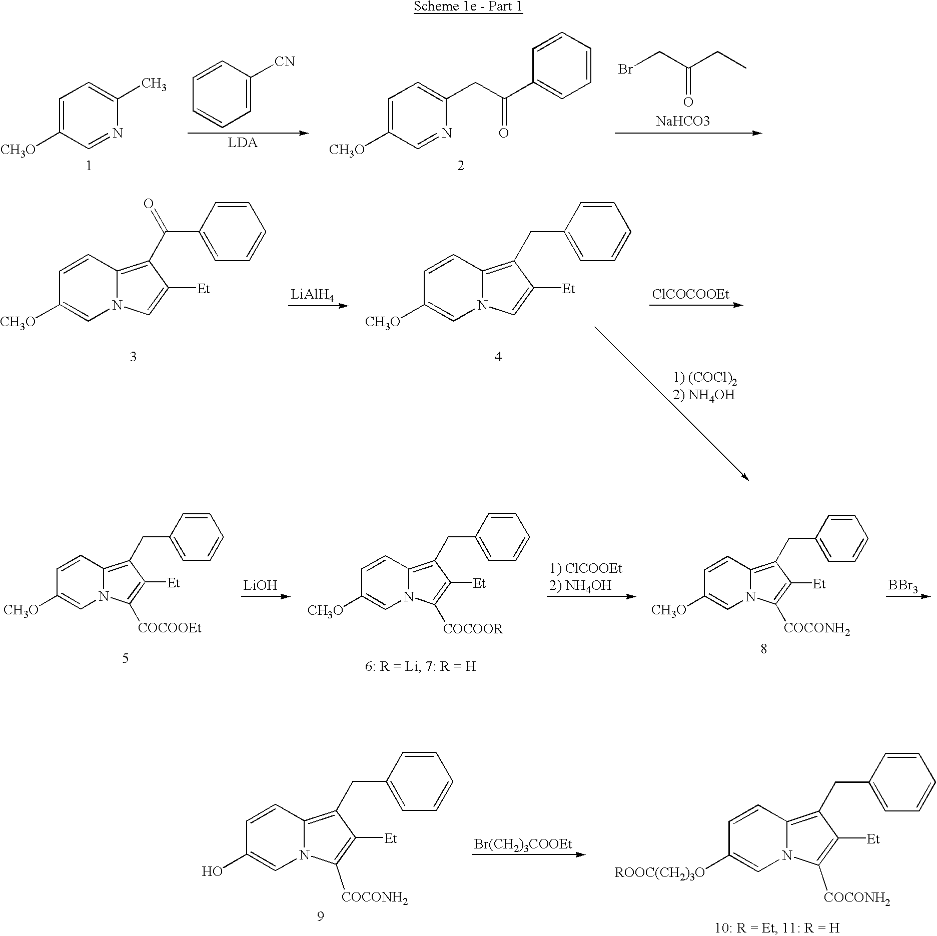

The anion of 2-methyl-5-methoxypyridine is formed in THF using lithium diisopropyl amide and reacted with benzonitrile to produce 2. Alkylation of the nitrogen of 2tby 1-bromo-2-butanone followed by base catalyzed cyclization forms 3 which is reduced by LAH to 4. Sequential treatment of 4 with oxalyl chloride and ammonia gives 8. Alternatively, 4 is acylated with ethyl oxalyl chloride to give 5 which is converted to 6 with lithium hydroxide and then to 8 by sequential treatment with ethyl chloroformate and ammonium hydroxide. Demethylation of 8 by BBr₃ yields 9 which is O-alkylated using base and ethyl 4-bromobutyrate to form 10. Hydrolysis of 10 by aqueous base produces 11.

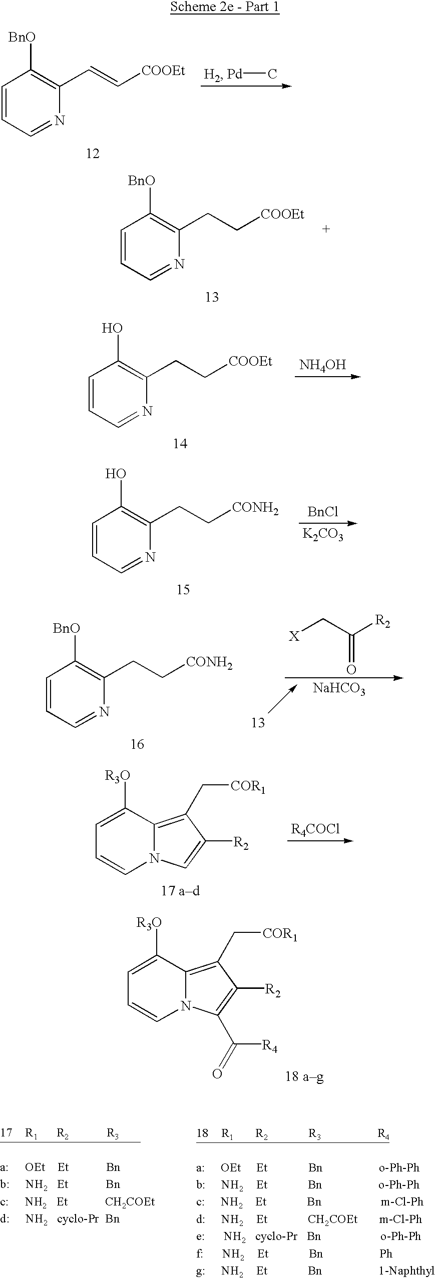

Compound 12 (N. Desidiri, A. Galli, I. Sestili, and M. L. Stein, Arch. Pharm. (Weinheim) 325, 29, (1992)) is reduced by hydrogen in the presence of Pd/C to 14 which 10 gives 15 on ammonolysis using ammonium hydroxide. O-alkylation of 15 using benzyl chloride and base affords 16. Alkylation of the nitrogen atom of 13 or 16 by 1-bromo-2-ketones followed by base catalyzed cyclization yields indolizines 17 which are acylated by aroyl halides to form 18.

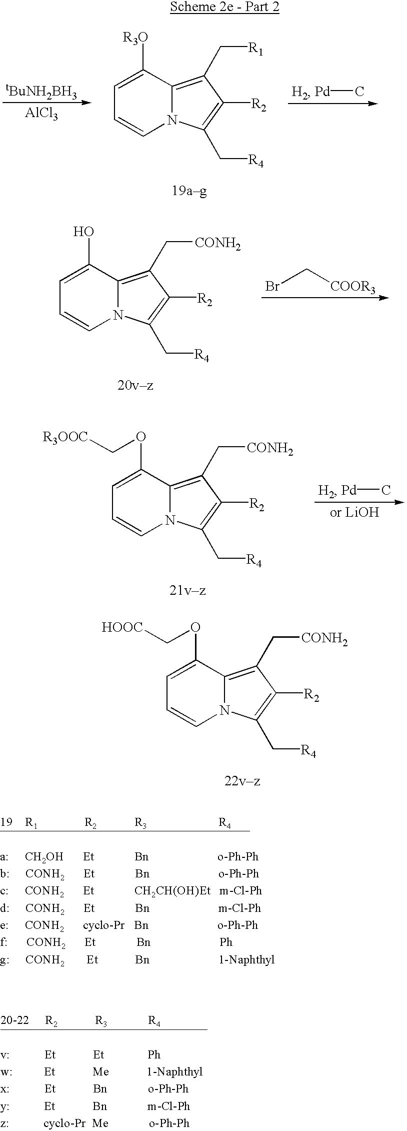

Reduction of 18 by tert-butylamine-borohydride and aluminum chloride yields 19 which is reduced by hydrogen in the presence of Pd/C to give 20. O-alkylation of 20 by benzyl bromoacetate and base forms 21 which is converted to the acid 22 by debenzylation using hydrogen in the presence of Pd/C.

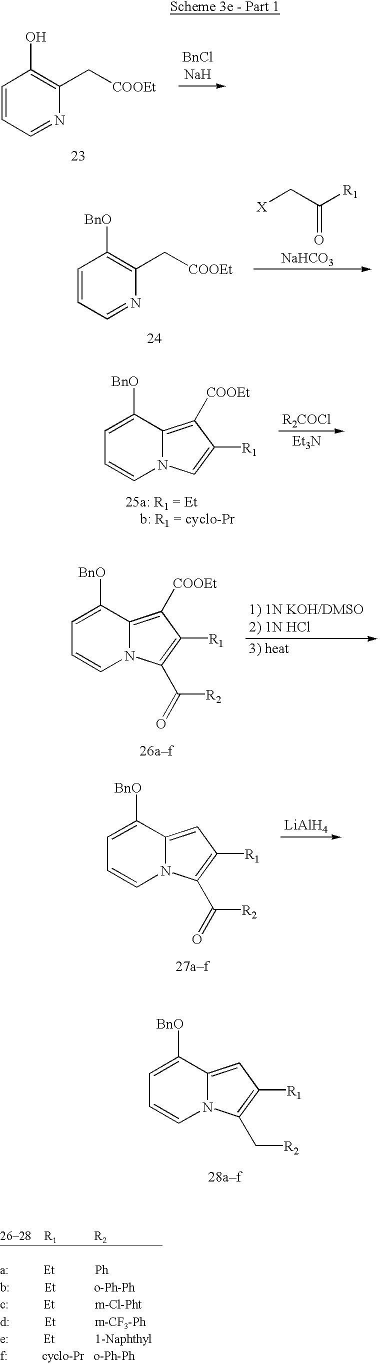

Compound 23 (N. Desideri F. Manna, M. L. Stein, G. Bile, W. Filippeelli, and E. Marmo, Eur. J. Med. Chem. Chim. Ther., 18, 295, (1983)) is O-alkylated using sodium hydride and benzyl chloride to give 24. N-alkylation of 24 by 1-bromo-2-butanone or chloromethylcyclopropyl ketone and subsequent base catalyzed cyclization gives 25 which is acylated by aroyl halide to give 26. Hydrolysis of the ester function of 26 followed by acidification forms an acid which is thermally decarboxylated to give 27. Reduction of the ketone function of 27 by LAH yields indolizines 28.

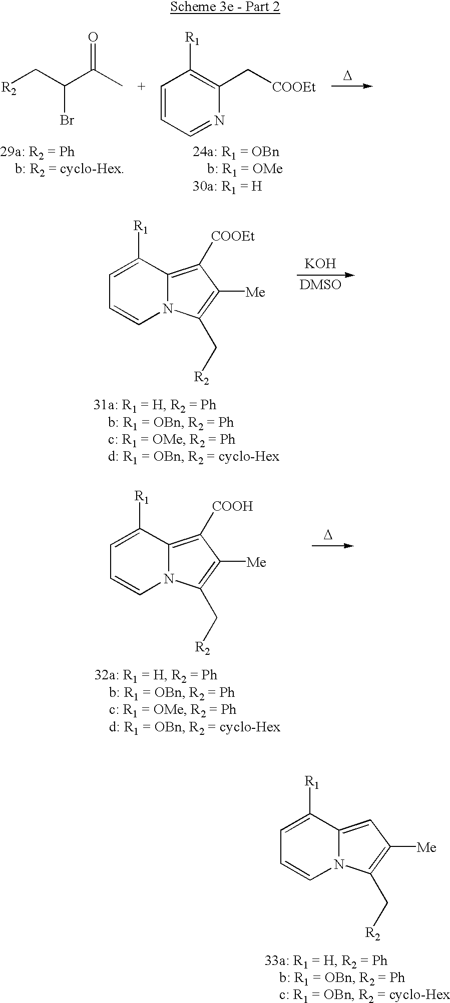

Heating a mixture of 3-bromo-4-phenyl-butan-2-one or 3-bromo-4-cyclohexyl-butan-2-one and ethyl pyridine-2-acetate, or a substituted derivative, in the presence of base yields indolizine 31. Treatment of 31 with aqueous base in DMSO at elevated temperature followed by acidification gives 32 which is thermally decarboxylated to 33.

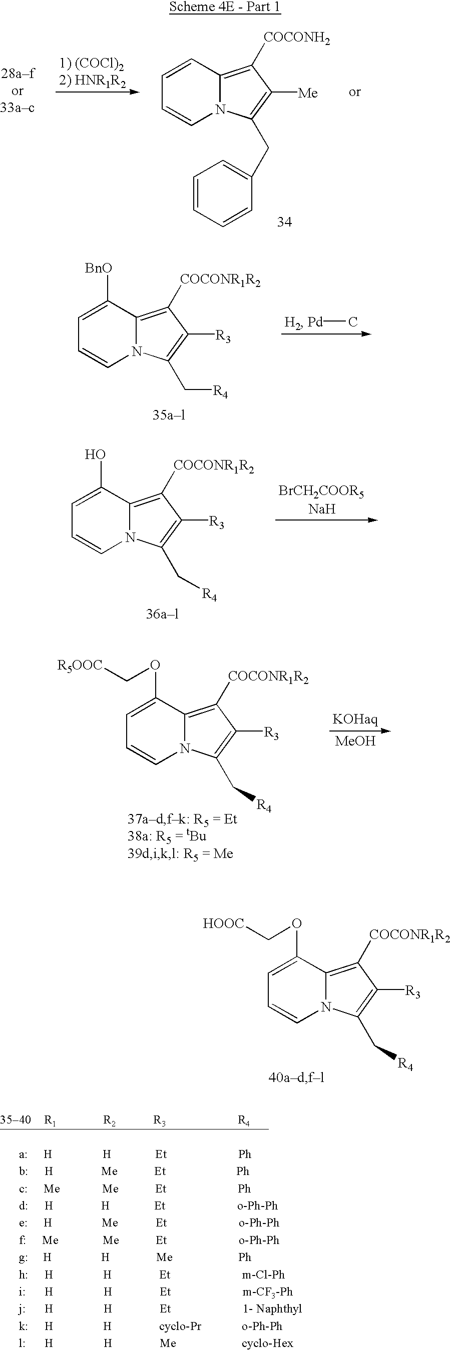

Sequential treatment of 28 or 33 with oxalyl chloride and ammonium hydroxide forms 35 which is debenzylated by hydrogen in the presence of Pd/C to give 36. Indolizines 36 are O-alkylated using sodium hydride and bromoacetic acid esters to form 37, 38, or 39 which are converted to indolizines 40 by hydrolysis with aqueous base followed by acidification.

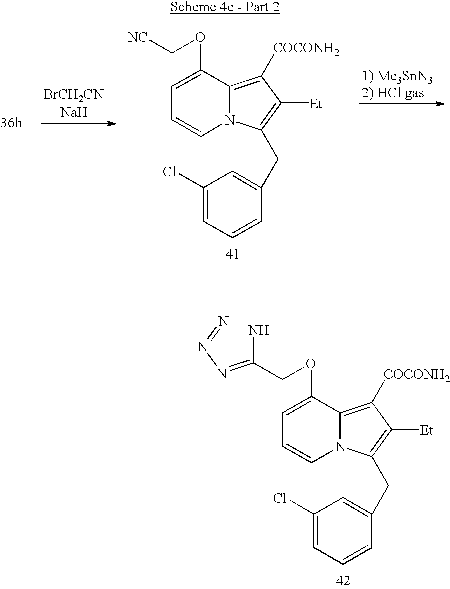

The O-alkylation of 36 h produces nitrite 41 which is converted to 42 on reaction with trialkyltin azide.

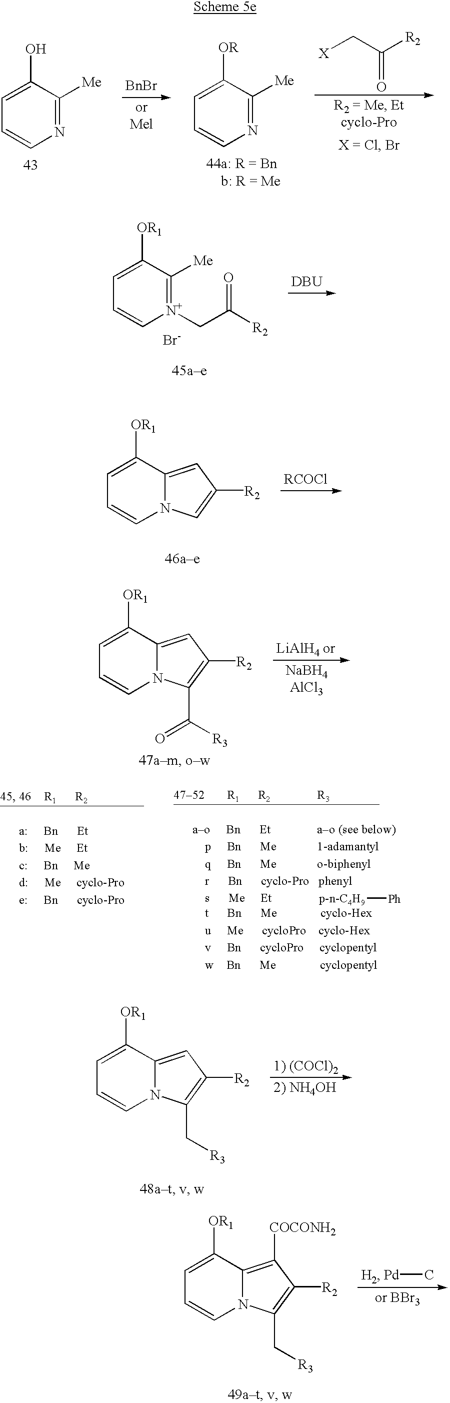

The hydroxypyridine is O-alkylated to give 44 which is heated with 2-haloketones to produce 45. Treatment of 45 with base causes cyclization to 46 which on heating with acid chlorides yields acylindolizines 47 which are reduced by aluminum hydride to the corresponding alkylindolizines 48. Sequential treatment of 48 with oxalyl chloride and then ammonia gives 49. Cleavage of the ether functionality of 49 yields 50. The oxyacetic ester derivatives 51 are formed by O-alkylation of 50 and then hydrolyzed to the oxyacetic acids 52.

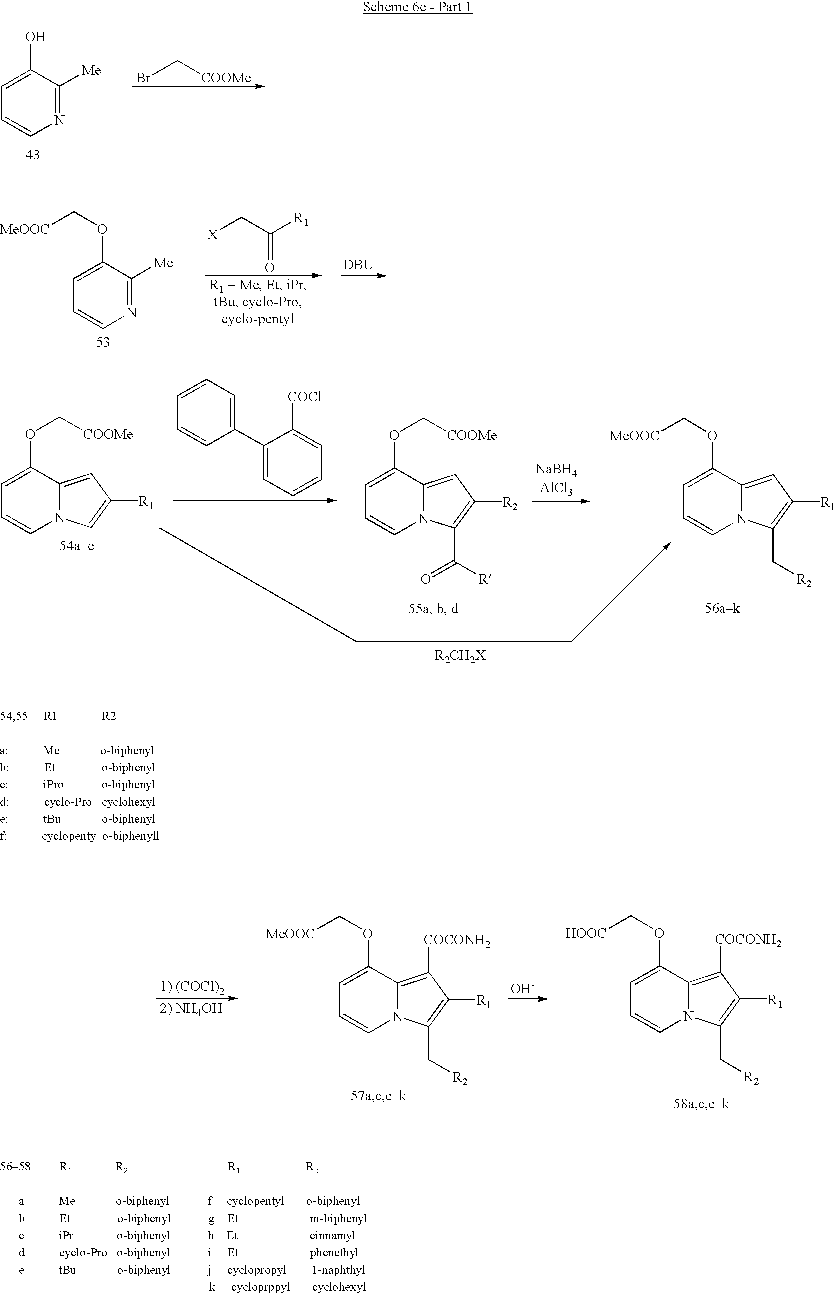

Pyridine 43 is O-alkylated to produce 53. Heating 53 with 2-haloketones gives intermediate N-alkylated pyridinium compounds which are cyclized to 54 on treatment with base. Heating 54 with acyl chlorides gives the acylindolizines 55 which are reduced to the alkylindolizines 56 by sodium borohydride-aluminum chloride. Alternatively, 56 are produced by C-alkylation of 54 using alkyl halides. Sequential treatment of 56 with oxalyl chloride and then ammonia gives 57 which are hydrolyzed to produce 58. Compound 58b is converted to its sodium salt 59a which yields 59b-k on reaction with the appropriate alkyl halide.

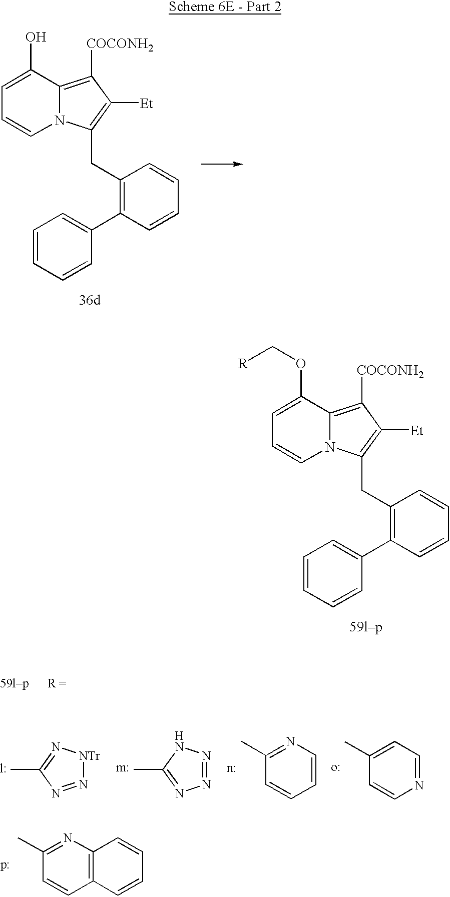

Compound 36b is O-alkylated to give 591-p.

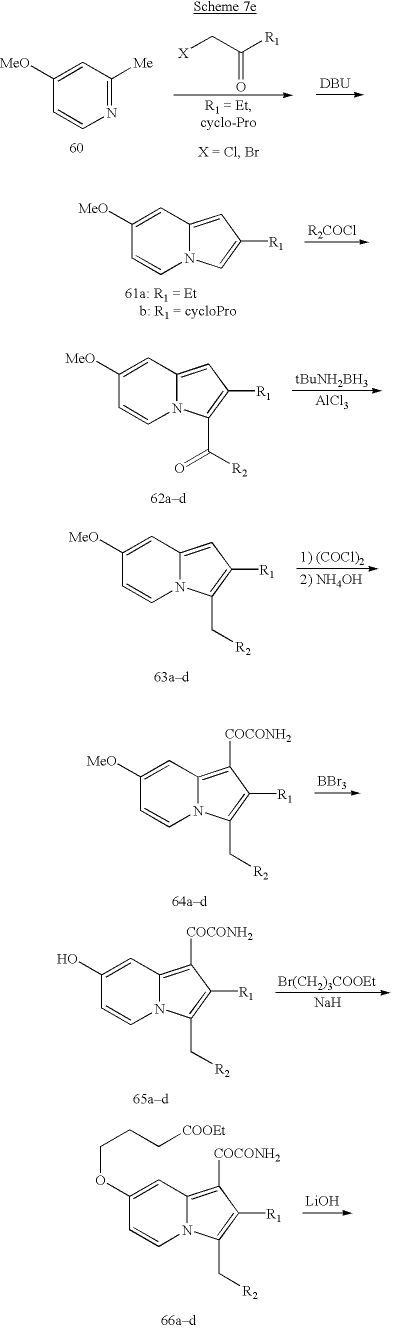

Pyridine 60 is N-alkylated by 2-haloketones to produce intermediate pyridinium compounds which are cyclized by base to give 61. Reaction of 61 with acyl chlorides produces 62 which are reduced to 63 by tert butylamine-borane and aluminum chloride. Sequential treatment of 63 with oxalyl chloride and then ammonia yields 64 which are O-demethylated by BBr₃ to give 65. The sodium salt of 65 is reacted with ethyl 4-bromobutyrate to give 66 which is hydrolyzed to the acid 67.

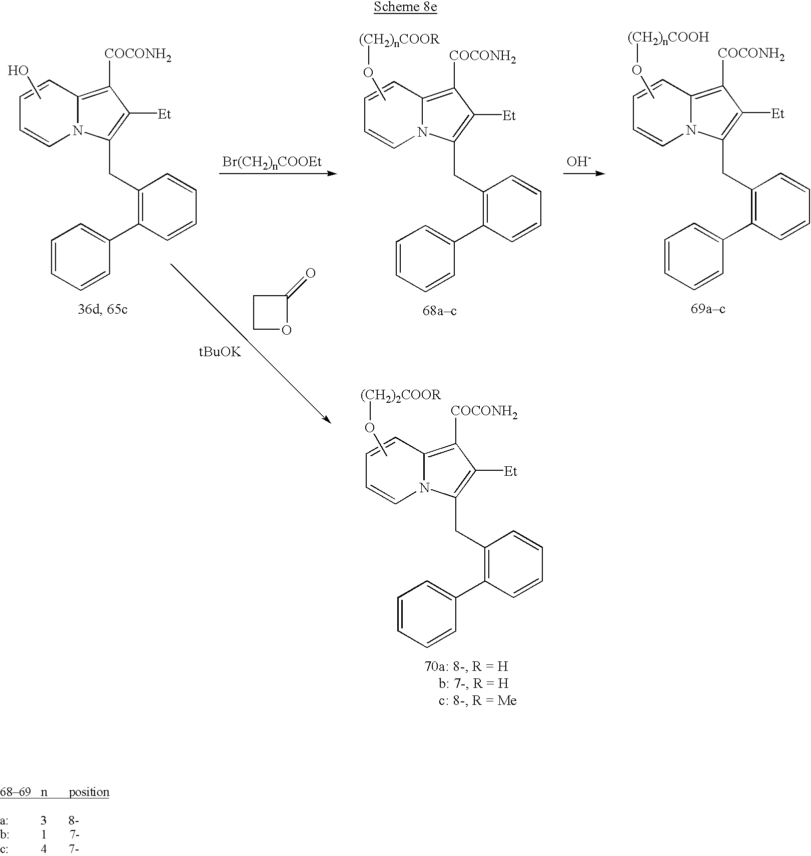

Compounds 36d and 65c are O-alkylated by omega-bromocarboxylic esters to give 68 which are hydrolyzed to the acids 69. Compounds 36d and 65c produce 70 on treatment with propiolactone and base.

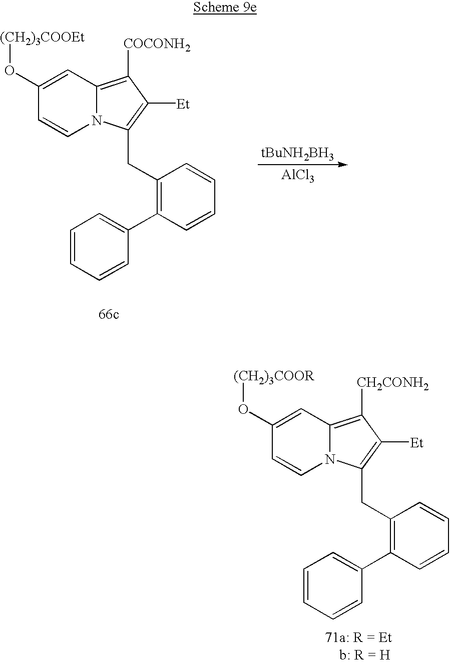

Compounds 66 are reduced to 71 by tert-butylamine-borane and aluminum chloride.

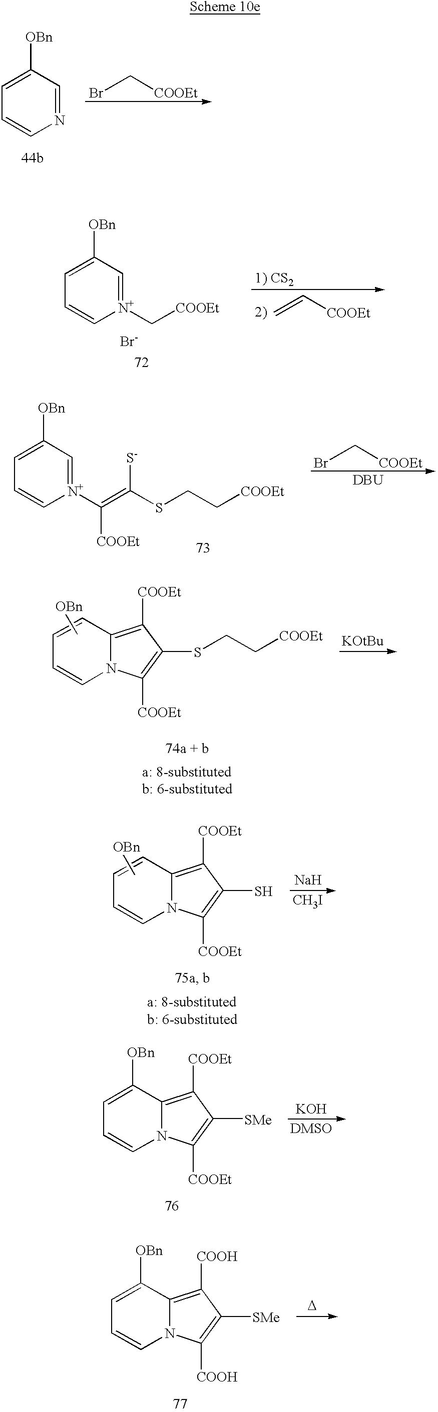

Pyridine 44b reacts with ethyl bromoacetate to produce 72 which is treated with CS₂ and base and then with ethyl acrylate to form 73. Reaction of 73 with base and ethyl bromoacetate yields a mixture of regioisomers 74a+b, 6- and 8-benzyloxy compounds. Base treatment of 74a+b eliminates ethyl acrylate to form 75 which is separated from the isomer of 6-benzyloxy derivative and S-alkylated to give 76. Hydrolysis of 76 forms 77 which is thermally decarboxylated to yield 78. Compound 78 is C-alkylated to form 79 which on sequential treatment with oxalyl chloride and then ammonia forms 80. Ether cleavage of 80 gives 81 whose sodium salt is alkylated by methyl bromoacetate to form 82 which are hydrolyzed to acids 83.

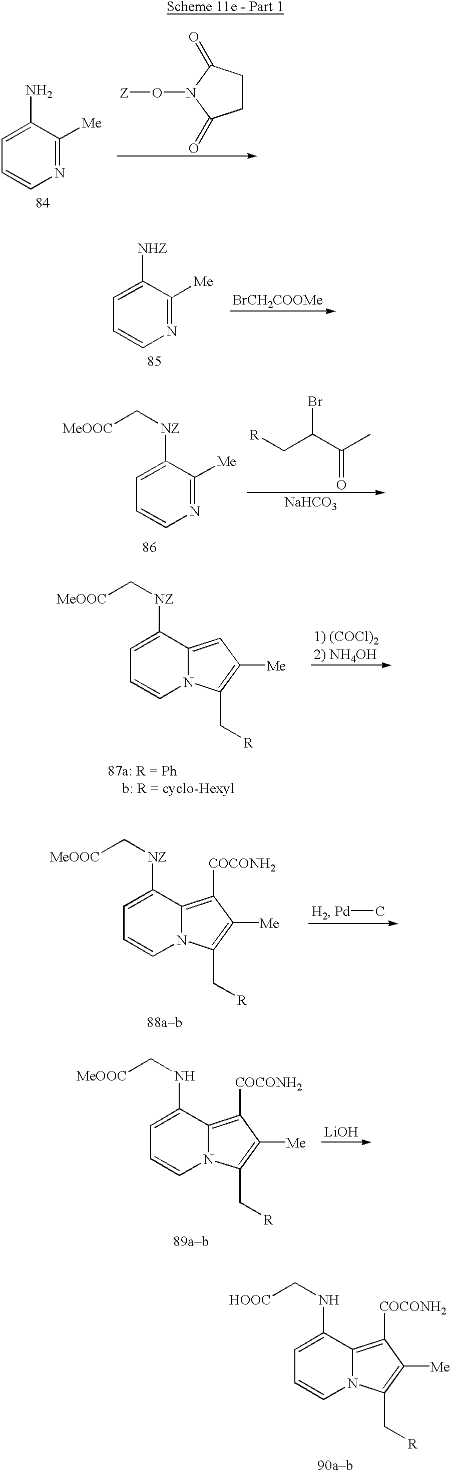

Aminopicoline 84 is converted to its N-CBZ derivative 85 whose anion is alkylated by methyl bromoacetate to produce 86. Reaction of 86 with methyl alpha-bromoalkyl ketones in the presence of base yields 87. Sequential treatment of 87 with oxalyl chloride and then ammonia gives 88 which is converted to 89 by hydrogenolysis of the N-CBZ function. Hydrolysis of 89 yields acids 90.

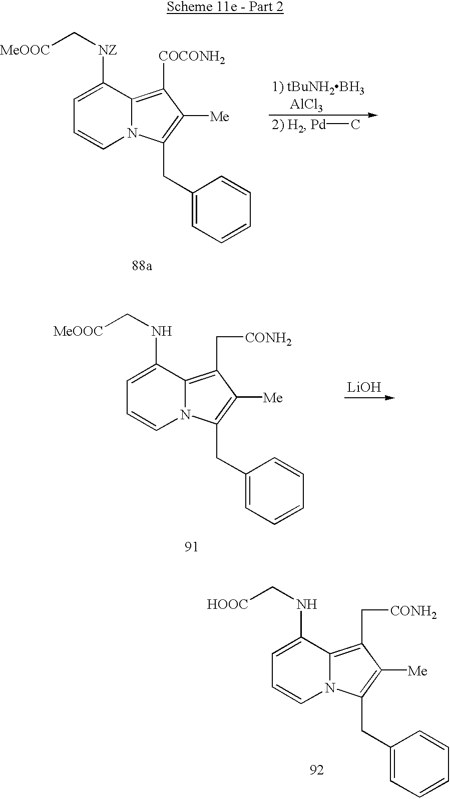

Compounds 88 are reduced by tert-butylamine-borane and aluminum chloride to 91 which are hydrolyzed to acids 92.

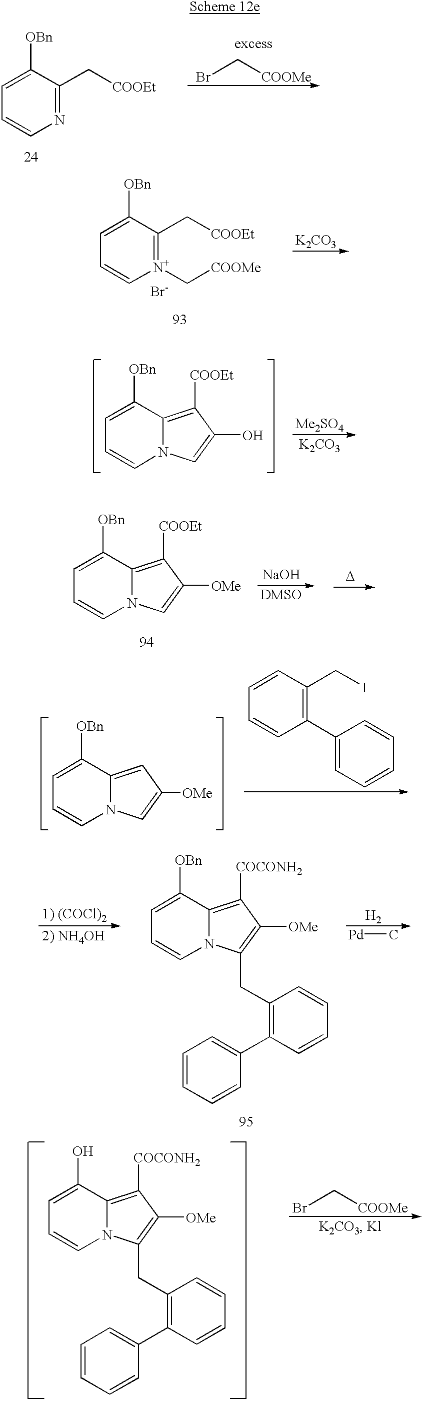

Pyridine 24 is N-alkylated by methyl bromoacetate, cyclized with base, and o-methylated using dimethysulfate to give 94. Hydrolysis of the ester function of 94 followed by thermal decarboxylation yields 2-methoxy-8-benzyloxyindolizine which is C-alkylated at position 3 and then reacted sequentially with oxalyl chloride and ammonia to produce 95. Hydrogenolysis of the 8-benzyloxy group followed by O-alkylation gives 96 which is hydrolyzed to 97.

f) Indene sPLA₂ inhibitors as described in U.S. patent application Ser. No. 08/776,618 filed Jul. 20, 1995, (titled, Synovial Phospholipase A₂ Inhibitor Compounds having an Indene Type Nucleus, Pharmaceutical Formulations Containing said Compounds, and Therapeutic Methods of Using Said Compounds”), the entire disclosure of which is incorporated herein by reference. These inhibitors are useful in making the compositions of the invention and practicing the method of the invention for the treatment of sepsis.

The method of the invention is for treatment of a mammal, including a human, afflicted with sepsis, said method comprising administering to said human a therapeutically effective amount of an indene-1-acetamide compound or a pharmaceutically acceptable salt, solvate or prodrug derivative thereof; wherein said compound is represented by the formula (If);

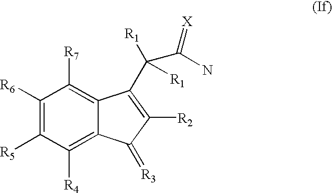

wherein;

X is oxygen or sulfur;

each R₁ is independently hydrogen, C₁-C₃ alkyl, or halo;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₆ and R₇ are independently selected from hydrogen, a non-interfering substituent, or the group, -(La)-(acidic group); wherein -(La)-, is an acid linker having an acid linker length of 1 to 10; provided, that at least one of R₆ and R₇ must be the group, -(La)-(acidic group); and

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

Suitable indene compounds also include the following:

An indene-1-acetic acid hydrazide compound or a pharmaceutically acceptable salt, solvate or prodrug derivative thereof; wherein said compound is represented by the formula (IIf);

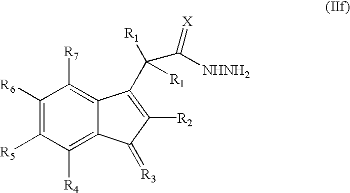

wherein:

X is oxygen or sulfur;

each R₁ is independently hydrogen, C₁-C₃ alkyl, or halo;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₆ and R₇ are independently selected from hydrogen, a non-interfering substituent, or the group, -(La)-(acidic group); wherein -(La)-, is an acid linker having an acid linker length of 1 to 10; provided, that at least one of R₆ and R₇ must be the group, -(La)-(acidic group); and

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

Suitable indene compounds for use in the method of the invention also include the following:

An indene-1-glyoxylamide compound or a pharmaceutically acceptable salt, solvate or prodrug derivative thereof; wherein said compound is represented by the formula (IIIf);

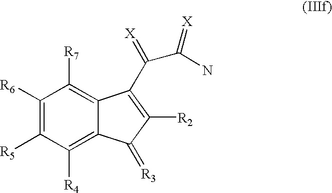

X is oxygen or sulfur;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, -O-(C₁-C₂ alkyl), -S-(C₁-C₂ alkyl), or a non-interfering substituent having a total of 1 to 3 atoms other than hydrogen;

R₆ and R₇ are independently selected from hydrogen, a non-interfering substituent, or the group, -(La)-(acidic group); wherein -(La)-, is an acid linker having an acid linker length of 1 to 10; provided, that at least one of R₆ and R₇ must be the group, -(Lₐ)-(acidic group);

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

The method of making the indene compounds is as follows:

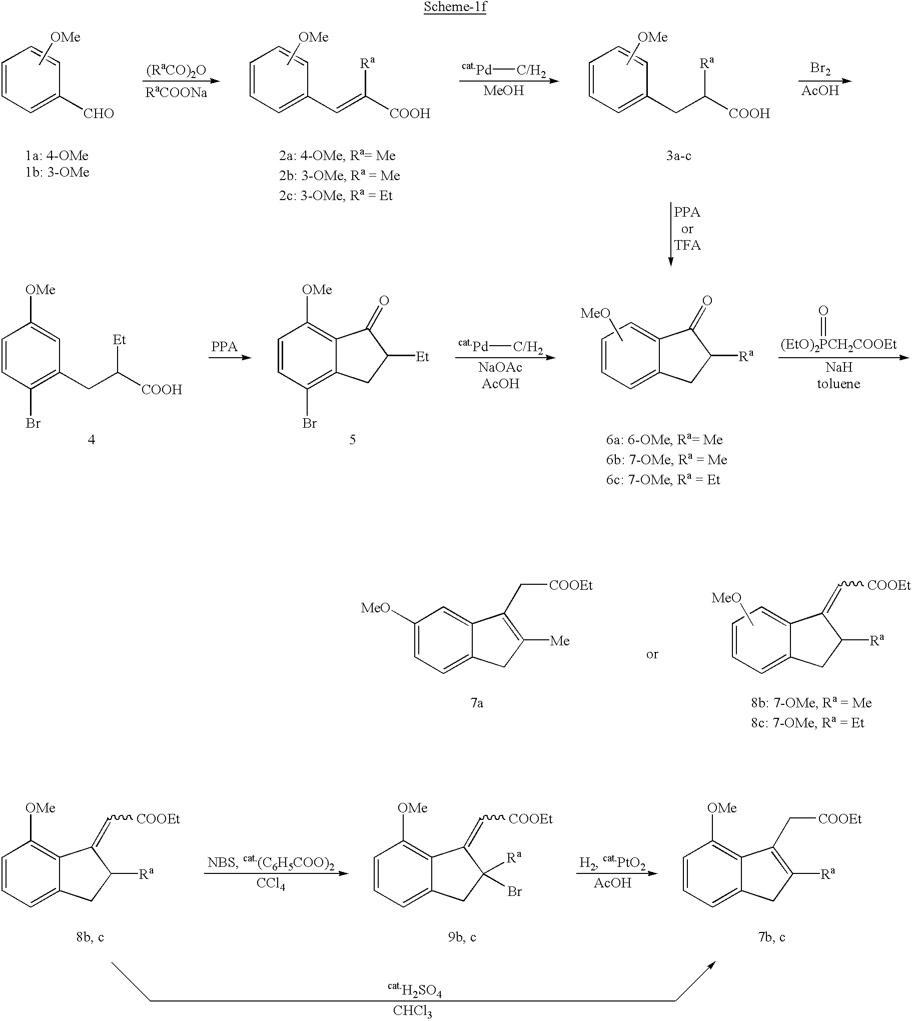

A mixture of an anisaldehyde 1, propionic anhydride, and sodium propionate is heated to produce 2 which is reduced by hydrogen in the presence of Pd/C to give 3. Acid cyclization of 3 yields 6. Alternatively, the aromatic position para to the methoxy group of 3 is blocked by bromination to give 4 which is cyclized to 5 by acid and then debrominated using hydrogen and Pd/C to give 6. Reaction of 6 with the anion of triethyl phosphonoacetate produces 7 and/or 8. Radical bromination of 8 gives 9, which on reduction with hydrogen in the presence of PtO₂ yields 7. Alternatively, treatment of 8 with acid gives 7.

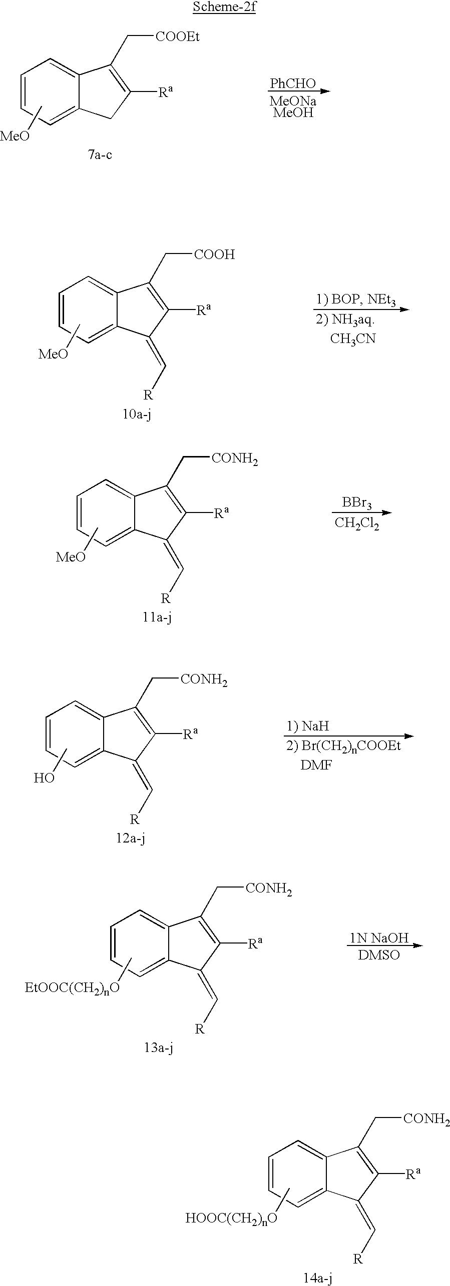

Compound 7 is condensed with benzaldehyde and its derivatives in the presence of base to give 10. Indenes 10 are converted to an active ester using benzotriazo-1-yloxytris(dimethylamino) hexafluorophosphonate and then reacted with ammonium hydroxide to form 11. Demethylation of 11 with BBr₃ forms 12 which is O-alkylated using sodium hydride and an omega-bromoalkanoic acid ester to produce 13. Aqueous base hydrolysis of 13 yields 14.

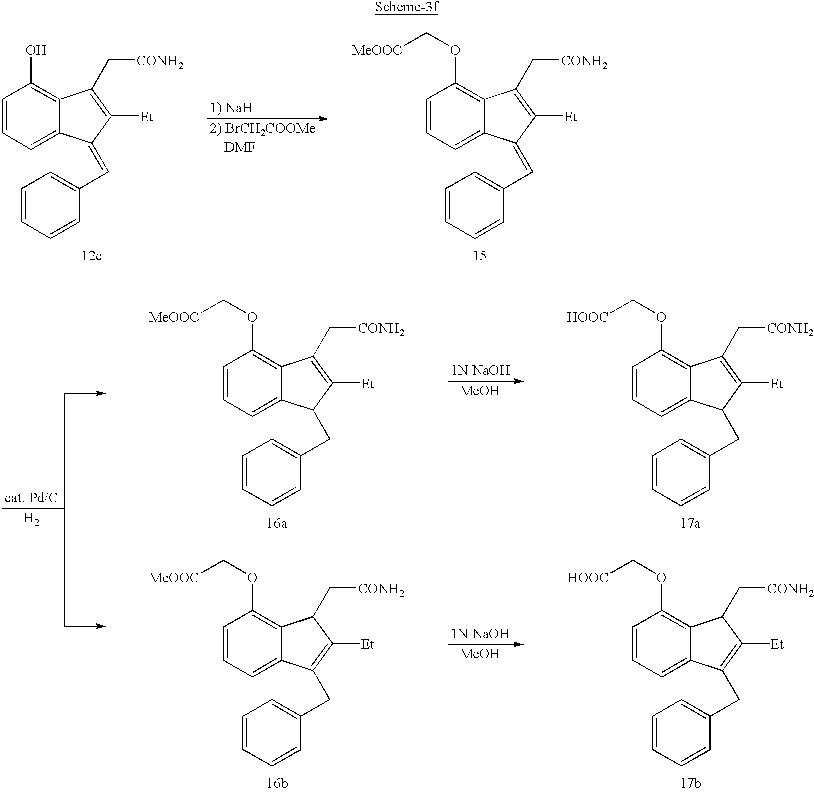

Compound 12c is O-alkylated using sodium hydride and methylbromoacetate to product 15 which is reduced by hydrogen in the presence of Pd/C to give a mixture of isomers 16a and 16b. Aqueous base hydrolysis of 16a and 16b gives 17a and 17b, respectively.

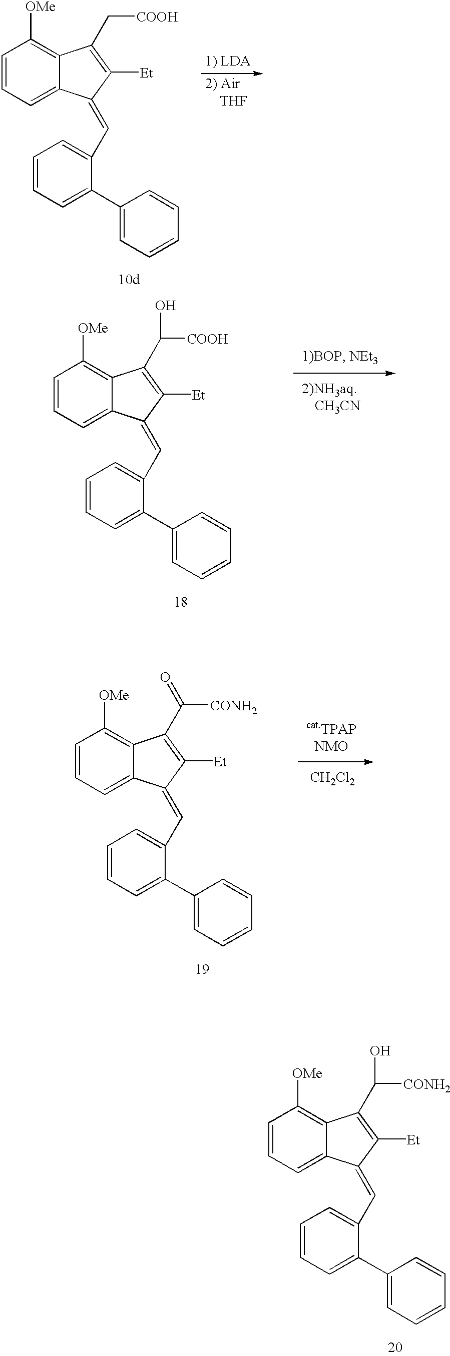

Compound 10d is treated with lithium diisopropylamine, then air is bubbled into the solution to give 18. The indene 18 is converted to an active ester using benzotriazo-1-yloxytris(dimethylamino)hexafluorophosphonate and then reacted with ammonium hydroxide to form the hydroxy acetamide 19. Compound 19 is oxidized to 20 using N-methylmorpholine N-oxide in the presence of tetrapropylammonium perruthenate.

g) Carbazole and tetrahydrocarbazole sPLA₂ inhibitors and methods of making these compounds are set out in U.S. patent application Ser. No. 09/063,066 filed Apr. 21, 1998 (titled, “Substituted Carbazoles and 1,2,3,4-Tetrahydrocarbazoles”), the entire disclosure of which is incorporated herein by reference. These inhibitors are useful in making the compositons of the invention and practicing the method of the invention for treating a mammal affliced with sepsis.

Useful carbazole or tetrahydrocarbazole inhibitors are represented by the following formulae:

A compound of the formula (Ie)

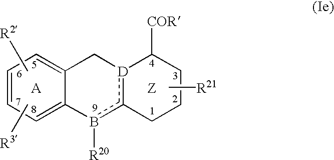

wherein;

A is phenyl or pyridyl wherein the nitrogen is at the 5-, 6-, 7- or 8-position;

one of B or D is nitrogen and the other is carbon;

Z is cyclohexenyl, phenyl, pyridyl, wherein the nitrogen is at the 1-, 2-, or 3-position, or a 6-membered heterocyclic ring having one heteroatom selected from the group consisting of sulfur or oxygen at the 1-, 2- or 3-position, and nitrogen at the 1-, 2-, 3- or 4-position; [symbol] is a double or single bond;

R²⁰ is selected from groups (a), (b) and (c) where;

(a) is —(C₅-C₂₀)alkyl, —(C₅-C₂₀)alkenyl, —(C₅-C₂₀)alkynyl, carbocyclic radicals, or heterocyclic radicals, or

(b) is a member of (a) substituted with one or more independently selected non-interfering substituents; or

(c) is the group -(L)-R⁸⁰; where, -(L)- is a divalent linking group of 1 to 12 atoms selected from carbon, hydrogen, oxygen, nitrogen, and sulfur; wherein the combination of atoms in -(L)- are selected from the group consisting of (i) carbon and hydrogen only, (ii) one sulfur only, (iii) one oxygen only, (iv) one or two nitrogen and hydrogen only, (v) carbon, hydrogen, and one sulfur only, and (vi) and carbon, hydrogen, and oxygen only; and where R⁸⁰ is a group selected from (a) or (b);

R²¹ is a non-interfering substituent;

R1′ is —NHNH₂, —NH₂ or —CONH₂;

R2′ is selected from the group consisting of —OH, and —O(CH₂)ₜR⁵′ where

R⁵′ is H, —CN, —NH₂, —CONH₂, —CONR⁹R¹⁰ —NHSO₂R¹⁵; —CONHSO₂R¹⁵, where R¹⁵ is —(C₁-C₆)alkyl or —CF₃; phenyl or phenyl substituted with —CO₂H or —CO₂(C₁-C₄)alkyl; and -(La)-(acidic group), wherein -(La)- is an acid linker having an acid linker length of 1 to 7 and t is 1-5;

R³′ is selected from non-interfering substituent, carbocyclic radicals, carbocyclic radicals substituted with non-interfering substituents, heterocyclic radicals, and heterocyclic radicals substituted with non-interfering substituents; or a pharmaceutically acceptable racemate, solvate, tautomer, optical isomer, prodrug derivative or salt thereof;

provided that; when R³′ is H, R²⁰ is benzyl and m is 1 or 2; R^{2′} cannot be —O(CH₂)ₘH; and

provided that when D is nitrogen, the heteroatom of Z is selected from the group consisting of sulfur or oxygen at the l-, 2- or 3-position and nitrogen at the 1-, 2-, 3- or 4-position.

Preferred in the compositions and method of the invention are compounds represented by the formula (IIe):

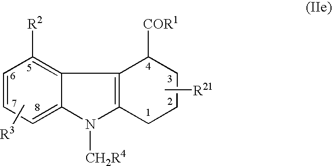

wherein;

Z is cyclohexenyl, or phenyl;

R²¹ is a non-interfering substituent;

R¹ is —NHNH₂ or —NH₂;

R² is selected from the group consisting of —OH and —O(CH₂)ₘ^{R5} where

R⁵ is H, —CO₂H, —CONH₂, —CO₂(C₁-C₄ alkyl);

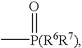

where R⁶ and R⁷ are each independently —OH or —O(C₁-C₄)alkyl; —SO₃H, —SO₃(C₁-C₄ alkyl), tetrazolyl, —CN, —NH₂, —NHSO₂R15; —CONHSO₂R15, where R15 is —(C₁-C₆)alkyl or —CF₃, phenyl or phenyl substituted with —CO₂H or —CO₂(C₁-C₄)alkyl where m is 1-3;

R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;

R⁴ is H, —(C₅-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl;

or a pharmaceutically acceptable racemate, solvate, tautomer, optical isomer, prodrug derivative or salt, thereof.

Preferred specific compounds including all salts and prodrug derivatives thereof, for the compositions and method of the invention are as follows:

9-benzyl-5,7-dimethoxy-1,2,3,4-tetrahydrocarbazole-4-carboxylic acid hydrazide;

9-benzyl-5,7-dimethoxy-1,2,3,4-tetrahydrocarbazole-4-carboxamide;

[9-benzyl-4-carbamoyl-7-methoxy-1,2,3,4-tetrahydrocarbazol-5-yl]oxyacetic acid sodium salt;

[9-benzyl-4-carbamoyl-7-methoxycarbazol-5-yl]oxyacetic acid;

methyl [9-benzyl-4-carbamoyl-7-methoxycarbazol-5-yl]oxyacetic acid;

9-benzyl-7-methoxy-5-cyanomethyloxy-1,2,3,4-tetrahydrocarbazole-4-carboxamide;

9-benzyl-7-methoxy-5-(1H-tetrazol-5-yl-methyl)oxy)-1,2,3,4-tetrahydrocarbazole-4-carboxamide;

{9-[(phenyl)methyl]-5-carbamoyl-2-methyl-carbazol-4-yl}oxyacetic acid;

{9-[(3-fluorophenyl)methyl]-5-carbamoyl-2-methyl-carbazol-4-yl}oxyacetic acid;

{9-[(3-methylphenyl)methyl]-5-carbamoyl-2-methyl-carbazol-4-yl}oxyacetic acid;

{9-[(phenyl)methyl]-5-carbamoyl-2-(4-trifluoromethylphenyl)-carbazol-4-yl}oxyacetic acid;

9-benzyl-5-(2-methanesulfonamido)ethyloxy-7-methoxy-1,2,3,4-tetrahydrocarbazole-4-carboxamide;

9-benzyl-4-(2-methanesulfonamido)ethyloxy-2-methoxycarbazole-5-carboxamide;

9-benzyl-4-(2-trifluoromethanesulfonamido)ethyloxy-2-methoxycarbazole-5-carboxamide;

9-benzyl-5-methanesulfonamidoylmethyloxy-7-methoxy-1,2,3,4-tetrahydrocarbazole-4-carboxamide;

9-benzyl-4-methanesulfonamidoylmethyloxy-carbazole-5-carboxamide;

[5-carbamoyl-2-pentyl-9-(phenylmethyl)carbazol-4-yl]oxyacetic acid;

[5-carbamoyl-2-(1-methylethyl)-9-(phenylmethyl)carbazol-4-yl]oxyacetic acid;

[5-carbamoyl-9-(phenylmethyl)-2-[(tri(-1-methylethyl)silyl)oxymethyl]carbazol-4-yl]oxyacetic acid;

[5-carbamoyl-2-phenyl-9-(phenylmethyl)carbazol-4-yl]oxyacetic acid[5-carbamoyl-2-(4-chlorophenyl)-9-(phenylmethyl)carbazol-4-yl]oxyacetic acid;

[5-carbamoyl-2-(2-furyl)-9-(phenylmethyl)carbazol-4-yl]oxyacetic acid;

[5-carbamoyl-9-(phenylmethyl)-2-[(tri(-1-methylethyl)silyl)oxymethyl]carbazol-4-yl]oxyacetic acid, lithium salt;

{9-[(phenyl)methyl]-5-carbamoylcarbazol-4-yl}oxyacetic acid;

{9-[(3-fluorophenyl)methyl]-5-carbamoylcarbazol-4-yl}oxyacetic acid;

{9-[(3-phenoxyphenyl)methyl]-5-carbamoylcarbazol-4-yl}oxyacetic acid;

{9-[(2-Fluorophenyl)methyl]-5-carbamoylcarbazol-4-yl}oxyacetic acid;

{9-[(2-trifluoromethylphenyl)methyl]-5-carbamoylcarbazol-4-yl}oxyacetic acid;

{9-[(2-benzylphenyl)methyl]-5-carbamoylcarbazol-4-yl}oxyacetic acid;

{9-[(3-trifluoromethylphenyl)methyl]-5-carbamoylcarbazol-4-yl}oxyacetic acid;

{9-[(1-naphthyl)methyl]-5-carbamoylcarbazol-4-yl}oxyacetic acid;

{9-[(2-cyanophenyl)methyl]-5-carbamoylcarbazol-4-yl}oxyacetic acid;

{9-[(3-cyanophenyl)methyl]-5-carbamoylcarbazol-4-yl}oxyacetic acid;

{9-[(2-methylphenyl)methyl]-5-carbamoylcarbazol-4-yl}oxyacetic acid;

{9-[(3-methylphenyl)methyl]-5-carbamoylcarbazol-4-yl}oxyacetic acid;

{9-[(3,5-dimethylphenyl)methyl]-5-carbamoylcarbazol-4-yl}oxyacetic acid;

{9-[(3-iodophenyl)methyl]-5-carbamoylcarbazol-4-yl}oxyacetic acid;

{9-[(2-Chlorophenyl)methyl]-5-carbamoylcarbazol-4-yl}oxyacetic acid;

{9-[(2,3-difluorophenyl)methyl]-5-carbamoylcarbazol-4-yl}oxyacetic acid;

{9-[(2,6-difluorophenyl)methyl]-5-carbamoylcarbazol-4-yl}oxyacetic acid;

{9-[(2,6-dichlorophenyl)methyl]-5-carbamoylcarbazol-4-yl}oxyacetic acid;

{9-[(3-trifluoromethoxyphenyl) methyl]-5-carbamoylcarbazol-4-yl}oxyacetic acid;

{9-[(2-biphenyl)methyl]-5-carbamoylcarbazol-4-yl}oxyacetic acid;

{9-[(2-Biphenyl)methyl]-5-carbamoylcarbazol-4-yl}oxyacetic acid;

the {9-[(2-Biphenyl)methyl]-5-carbamoylcarbazol-4-yl}oxyacetic acid;

[9-Benzyl-4-carbamoyl-1,2,3,4-tetrahydrocarbaole-5-yl]oxyacetic acid;

{9-[(2-Pyridyl)methyl]-5-carbamoylcarbazol-4-yl}oxyacetic acid;

{9-[(3-Pyridyl)methyl]-5-carbamoylcarbazol-4-yl}oxyacetic acid;

[9-benzyl-4-carbamoyl-8-methyl-1,2,3,4-tetrahydrocarbazol-5-yl]oxyacetic acid;

[9-benzyl-5-carbamoyl-1-methylcarbazol-4-yl]oxyacetic acid;

[9-benzyl-4-carbamoyl-8-fluoro-1, 2,3,4-tetrahydrocarbazol-5-yl]oxyacetic acid;

[9-benzyl-5-carbamoyl-1-fluorocarbazol-4-yl]oxyacetic acid;

[9-benzyl-4-carbamoyl-8-chloro-1,2,3,4-tetrahydrocarbazol-5-yl]oxyacetic acid;

[9-benzyl-5-carbamoyl-1-chlorocarbazol-4-yl]oxyacetic acid;

[9-[(Cyclohexyl)methyl]-5-carbamoylcarbazol-4-yl]oxyacetic acid;

[9-[(Cyclopentyl)methyl]-5-carbamoylcarbazol-4-yl]oxyacetic acid;

5-carbamoyl-9-(phenylmethyl)-2-[[(propen-3-yl)oxy]methyl]carbazol-4-yl]oxyacetic acid;

[5-carbamoyl-9-(phenylmethyl)-2-[(propyloxy)methyl]carbazol-4-yl]oxyacetic acid;

9-benzyl-7-methoxy-5-((carboxamidomethyl)oxy)-1,2,3,4-tetrahydrocarbazole-4-carboxamide;

9-benzyl-7-methoxy-5-cyanomethyloxy-carbazole-4-carboxamide;

9-benzyl-7-methoxy-5-((1H-tetrazol-5-yl-methyl)oxy)-carbazole-4-carboxamide;

9-benzyl-7-methoxy-5-((carboxamidomethyl)oxy)-carbazole-4-carboxamide; and

[9-Benzyl-4-carbamoyl-1,2,3,4-tetrahydrocarbaole-5-yl]oxyacetic acid

or a pharmaceutically acceptable racemate, solvate, tautomer, optical isomer, prodrug derivative or salt, thereof.

Other desirable carbazole inhibitors suitable for the compositions and method of thein invention are selected from those represented by the formula (XXX):

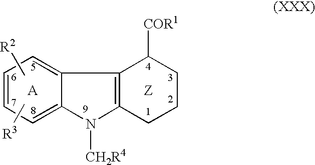

wherein:

R¹ is —NHNH₂, or —NH₂;

R² is selected from the group consisting of —OH and —O(CH₂)ₘR⁵ where

R⁵ is H, —CO₂H, —CO₂(C₁-C₄ alkyl);

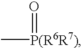

where R⁶ and R⁷ are each independently —OH or —O(C₁-C₄)alkyl; —SO₃H, —SO₃(C₁-C₄ alkyl), tetrazolyl, —CN, —NH₂ —NHSO₂R¹⁵; —CONHSO₂R¹⁵, where R¹⁵ is —(C₁-C₆)alkyl or —CF₃, phenyl or phenyl substituted with —CO₂H or —CO₂(C₁-C₄)alkyl where m is 1-3;

R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;

R⁴ is H, —(C₅-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl;

A is phenyl or pyridyl wherein the nitrogen is at the 5-, 6-, 7- or 8-position;

Z is cyclohexenyl, phenyl, pyridyl wherein the nitrogen is at the 1-, 2- or 3-position or a 6-membered heterocyclic ring having one heteroatom selected from the group consisting of sulfur or oxygen at the 1-, 2- or 3-position and nitrogen at the 1-, 2-, 3- or 4-position, or

wherein one carbon on the heterocyclic ring is optionally substituted with ═O;

or a pharmaceutically acceptable racemate, solvate, tautomer, optical isomer, prodrug derivative or salt thereof;

provided that one of A or Z is a heterocyclic ring.

Further desirable specific cabazole and tetrahydrocarbazole inhibitors suitable for the compositions and method of the invention are selected from the following:

(R,S)-(9-benzyl-4-carbamoyl-1-oxo-3-thia-1,2,3,4-tetrahydrocarbazol-5-yl)oxyacetic acid; (R,S)-(9-benzyl-4-carbamoyl-1-oxo-3-thia-1,2,3,4-tetrahydrocarbazol-5-yl)oxyacetic acid; [N-benzyl-1-carbamoyl-1-aza-1,2,3,4-tetrahydrocarbazol-8-yl]oxyacetic acid; 4-methoxy-6-methoxycarbonyl-10-phenylmethyl-6,7,8,9-tetrahydropyrido[1,2-a]indole; (4-carboxamido-9-phenylmethyl-4,5-dihydrothiopyrano[3,4-b]indol-5-yl)oxyacetic acid; 3,4-dihydro-4-carboxamidol-5-methoxy-9-phenylmethylpyrano[3,4-b]indole; 2-[(2,9 bis-benzyl-4-carbamoyl-1,2,3,4-tetrahydro-beta-carbolin-5-yl)oxy]acetic acid or a pharmaceutically acceptable racemate, solvate, tautomer, optical isomer, prodrug derivative or salt thereof.

The most preferred cabozole/tetrahydrocarbazole inhibitors for the compositions and method of treating sepsis are represented by the formulae (Xe) and (XIe) below:

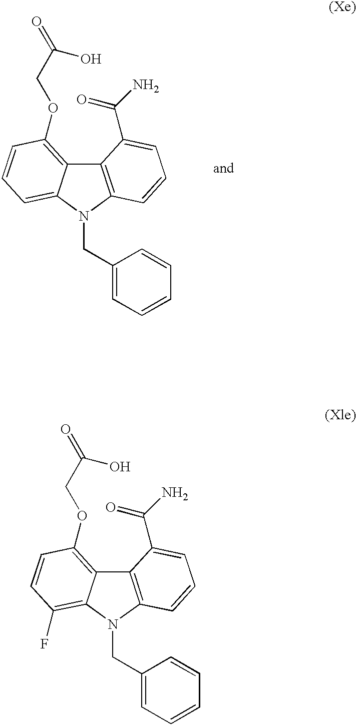

For all of the above compounds of the carbazole or tetrahydrocarbazole type it is advantageous to use them in their (i)acid form, or (ii) pharmaceutically acceptable (e.g., Na, K) form, or (iii) and prodrugs derivatives (e.g., methyl ester, ethyl ester, n-butyl ester, morpholino ethyl ester).

Prodrugs are derivatives of sPLA₂ inhibitors used in the method of the invention which have chemically or metabolically cleavable groups and become by solvolysis or under physiological conditions the compounds of the invention which are pharmaceutically active in vivo. Derivatives of the compounds of this invention have activity in both their acid and base derivative forms, but the acid derivative form often offers advantages of solubility, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Specific preferred prodrugs are ester prodrugs inclusive of methyl ester, ethyl ester, n-propyl ester, isopropyl ester, n-butyl ester, sec-butyl, tert-butyl ester, N,N-diethylglycolamido ester, and morpholino-N-ethyl ester. Methods of making ester prodrugs are disclosed in U.S. Pat. No. 5,654,326. Additional methods of prodrug synthesis are disclosed in U.S. Provisional Patent Application Serial No. 60/063,280 filed Oct. 27, 1997 (titled, N,N-diethylglycolamido ester Prodrugs of Indole sPLA2 Inhibitors), the entire disclosure of which is incorporated herein by reference; U.S. Provisional Patent Application Serial No. 60/063,646 filed Oct. 27, 1997 (titled, Morpholino-N-ethyl Ester Prodrugs of Indole sPLA2 Inhibitors), the entire disclosure of which is incorporated herein by reference; and U.S. Provisional Patent Application Serial No. 60/063,284 filed Oct. 27, 1997 (titled, Isopropyl Ester Prodrugs of Indole sPLA₂ Inhibitors), the entire disclosure of which is incorporated herein by reference.

Carbazole and tetrahydrocarbazole sPLA₂ inhibitor compounds useful for practicing the method of the invention may be made by the following general methods:

The compounds of formula Ie where Z is cyclohexene are prepared according to the following reaction Schemes Ig(a)and (c).

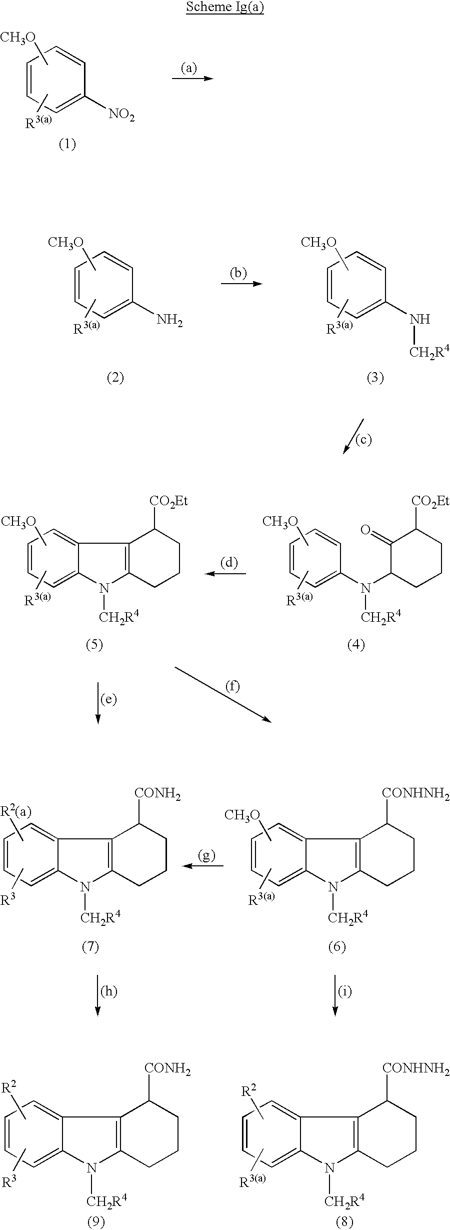

wherein;

R¹ is —NH₂, R³(a) is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁-C₆)alkenyl; or —(CH₂)ₙR⁸ where R⁸ is H, —CONH₂, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8;

when R¹ is —NHNH₂, R³(a) is H, —O(C₁-C₄)alkyl, halo, —(C₁-C₆)alkyl, phenyl, —(C₁-C₄)alkylphenyl; phenyl substituted with —(C₁-C₆)alkyl, halo or —CF_{3; —CH}₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁-C₆)alkenyl; or —(CH₂)ₙR⁸ where R⁸ is H, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8;

R^{2(a)} is —OCH₃ or —OH.

An appropriately substituted nitrobenzene (1) can be reduced to the aniline (2) by treatment with a reducing agent, such as hydrogen in the presence of Pd/C, preferably at room temperature.

Compound (2) is N-alkylated at temperatures of from about 0 to 20° C. using an alkylating agent such as an appropriately substituted aldehyde and sodium cyanoborohydride to form (3). Alternately, an appropriately substituted benzyl halide may be used for the first alkylation step. The resulting intermediate is further N-alkylated by treatment with 2-carbethoxy-6-bromocyclohexanone, preferably at temperatures of about 80° C. to yield (4) or by treatment with potassium hexamethyldisilazide and the bromoketoester.

The product (4) is cyclized to the tetrahydrocarbazole (5) by refluxing with ZnCl₂ in benzene for from about 1 to 2 days, preferably at 80° C. (Ref 1). Compound (5) is converted to the hydrazide (6) by treatment with hydrazine at temperatures of about 100° C., or to the amide (7) by reacting with methylchloroaluminum amide in benzene. (Ref 2) Alternatively, (7) may be produced by treatment of (6) with Raney nickel active catalyst.

It will be readily appreciated that when R^{3(a)} is:

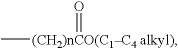

conversion to the amide will also be achieved in this procedure.

Compounds (6) and (7) may be dealkylated, preferably at 0° C. to room temperature, with a dealkylating agent, such as boron tribromide or sodium thioethoxide, to give compound (7) where R^{2(a)} is —OH, which may then be further converted to compound (9), by realkylating with a base, such as sodium hydride, and an alkylating agent, such as Br(CH₂)ₘR⁵, where R⁵ is the carboxylate or phosphonic diester or nitrile as defined above. Conversion of R² to the carboxylic acid may be accomplished by treatment with an aqueous base. When R² is nitrile, conversion to the tetrazole may be achieved by reacting with tri-butyl tin azide or conversion to the carboxamide may be achieved by reacting with basic hydrogen peroxide. When R² is the phosphonic diester, conversion to the acid may be achieved by reacting with a dealkylating agent such as trimethylsilyl bromide. The monoester may be accomplished by reacting the diester with an aqueous base.

When R² and R³ are both methoxy, selective demethylation can be achieved by treating with sodium ethanethiolate in dimethylformamide at 100° C.

Ref 1 Julia, M.; Lenzi, J. Preparation d'acides tetrahydro-1,2,3,4-carbazole-1 ou-4. Bull.Soc.Chim.France, 1962, 2262-2263.

Ref 2 Levin, J. I.; Turos, E.; Weinreb, S. M. An alternative procedure for the aluminum-mediated conversion of esters to amides. Syn. Comm., 1982, 12, 989-993.

An alternative synthesis of intermediate (5) is shown in Scheme I(b), as follows.

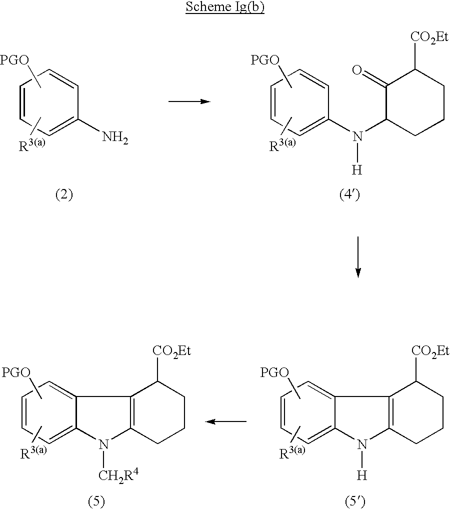

where PG is a protecting group;

R^{3a} is as defined in Scheme 1, above.

The aniline (2) is N-alkylated with 2-carbethoxy-6-bromocyclohexanone in dimethyl formamide in the presence of sodium bicarbonate for 8-24 hours at 50° C. Preferred protecting groups include methyl, carbonate, and silyl groups, such as t-butyldimethylsilyl. The reaction product (4′) is cyclized to (5′) using the ZnCl₂ in benzene conditions described in Scheme I(a), above. N-alkylation of (5′) to yield (5) is accomplished by treatment with sodium hydride and the appropriate alkyl halide in dimethylformamide at room temperature for 4-8 hours.

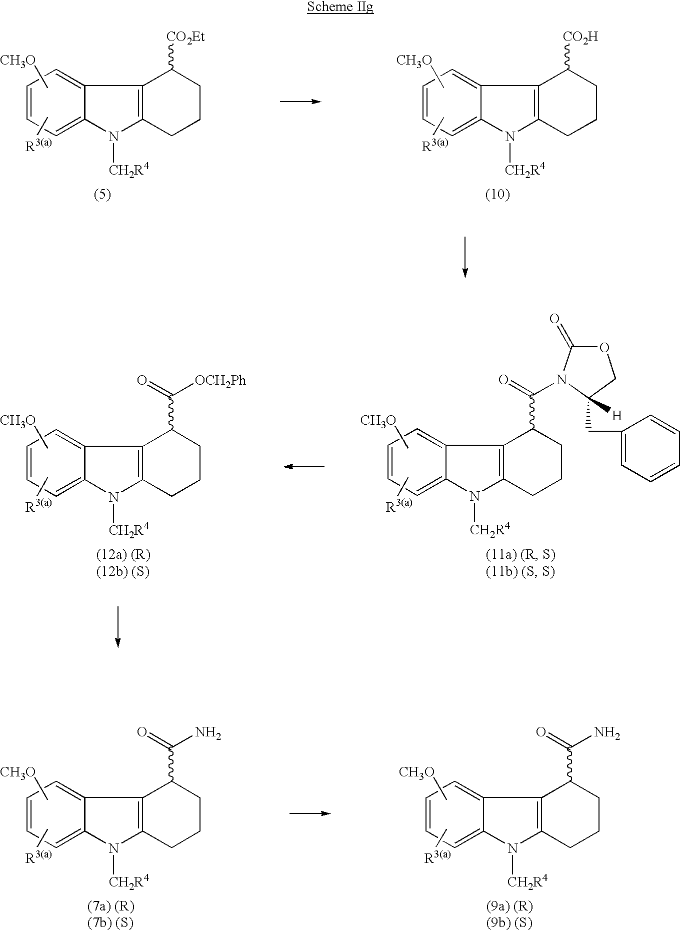

R^{3(a)} is as defined in Scheme Ig.

As discussed in Scheme I above, carbazole (5) is hydrolyzed to the carboxylic acid (10) by treatment with an aqueous base, preferably at room temperature to about 100° C. The intermediate is then converted to an acid chloride utilizing, for example, oxalyl chloride and dimethylformamide, and then further reacted with a lithium salt of (S) or (R)-4-alkyl-2-oxazolidine at a temperature of about −75° C., to give (11a) and (11b), which are separable by chromatography.

The diastereomers are converted to the corresponding enantiomeric benzyl esters (12) by brief treatment at temperatures of about 0° C. to room temperature with lithium benzyl oxide. (Ref 3) The esters (12) are then converted to (7) preferably by treatment with methylchloroaluminum amide (Ref 2, above) or, alternately, by hydrogenation using, for example, hydrogen and palladium on carbon, as described above, to make the acid and then reacting with an acyl azide, such as diphenylphosphoryl azide followed by treatment with ammonia. Using the procedure described above in Scheme I, compound (9a) or (9b) may be accomplished.

Ref 3 Evans, D. A.; Ennis, M. D.; Mathre, D. J. Asymmetric alkylation reactions of chiral imide enolates. A practical approach to the enantioselective synthesis of alpha-substituted carboxylic acid derivatives. J. Am. Chem. Soc., 1982, 104, 1737-1738.

Compounds of formula Ie where Z is phenyl can be prepared as follows in Schemes III(a)-(f), below.

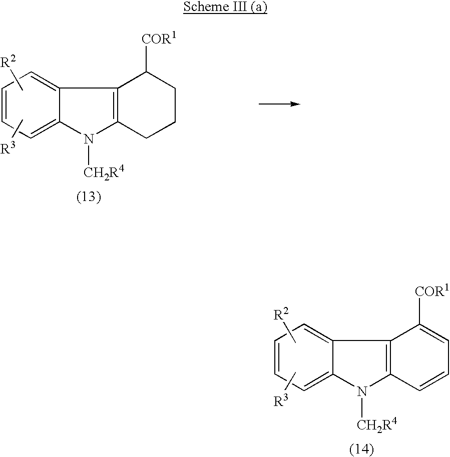

A 1,2,3,4-tetrahydrocarbazole-4-carboxamide or 4-carboxhydrazide (13) is dehydrogenated by refluxing in a solvent such as carbitol in the presence of Pd/C to produce the carbazole-4-carboxamide. Alternately, treatment of (13) with DDQ in an appropriate solvent such as dioxane yields carbozole (14).

Depending on the substituent pattern oxidation as described above may result in de-alkylation of the nitrogen. For example when R³ is substituted at the 8-position with methyl, oxidation results in dealkylation of the nitrogen which may be realkylated by treatment with sodium hydride and the appropriate alkyl halide as described in Scheme I(a) above to prepare the deired product (14).

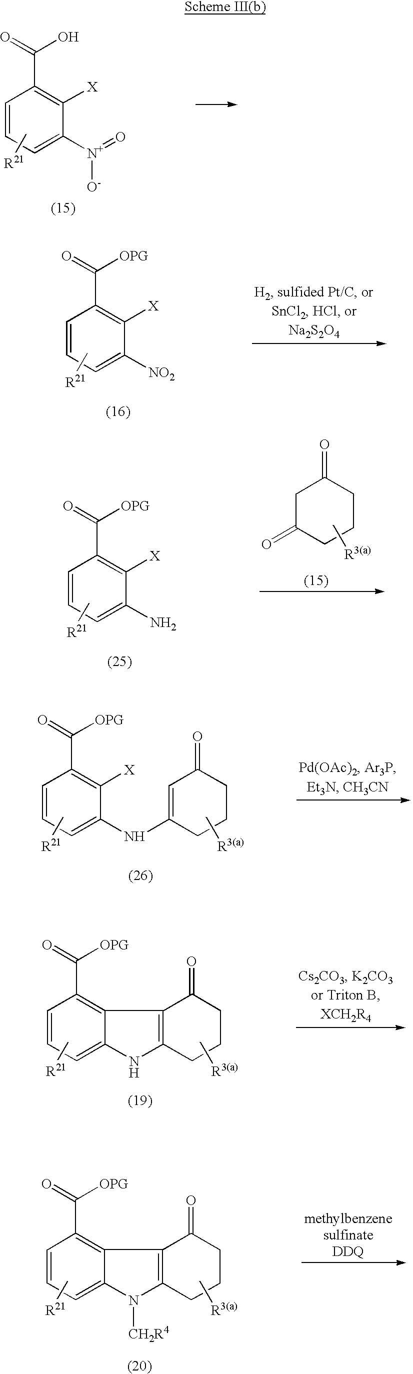

Benzoic acid derivative(16) where X is preferably chlorine, bromine or iodine and the protecting group is preferably —CH₃, are reduced to the corresponding aniline (25) with a reducing agent, such as stannous chloride in the presence of acid under the general conditions of Sakamoto et al, Chem Pharm. Bull. 35 (5), 1823-1828 (1987).

Alternatively, reduction with sodium dithionite in the presence of a base, such as sodium carbonate in a noninterferring solvent, such as water, ethanol, and/or tetrahydrofuran affords starting material (16).

Alternatively, reduction by hydrogenation over a sulfided platinum catalyst supported on carbon with hydrogen at 1 to 60 atmospheres in a noninterfering solvent, preferably ethyl acetate, to form a starting material (16).

The reactions are conducted at temperatures from about 0 to 100° C. preferably at ambient temperature, and are substantially complete in about 1 to 48 hours depending on conditions.

The aniline (25) and dione (15) are condensed under dehydrating conditions, for example, using the general procedure of Iida, et al., (Ref 5), with or without a noninterfering solvent, such as toluene, benzene, or methylene chloride, under dehydrating conditions at a temperature about 10 to 150° C. The water formed in the process can be removed by distillation, azetropic removal via a Dean-Stark apparatus, or the addition of a drying agent, such as molecular sieves, magnesium sulfate, calcium carbonate, sodium sulfate, and the like.

The process can be performed with or without a catalytic amount of an acid, such a p-toluenesulfonic acid or methanesulfonic acid. Other examples of suitable catalysts include hydrochloric acid, phenylsulfonic acid, calcium chloride, and acetic acid.

Examples of other suitable solvents include tetrahydrofuran, ethyl acetate, methanol, ethanol, 1,1,2,2-tetrachloroethane, chlorobenzene, bromobenzene, xylenes, and carbotetrachloride.

The condensation of the instant process is preferably carried out neat, at a temperature about 100 to 150° C. with the resultant water removed by distillation via a stream of inert gas, such as, nitrogen or argon.

The reaction is substantially complete in about 30 minutes to 24 hours.

Intermediate (26) may then be readily cyclized in the presence of a palladium catalyst, such as Pd(OAc)₂ or Pd(PPh₃)₄ and the like, a phosphine, preferably a trialkyl- or triarylphosphine, such as triphenylphosphine, tri-o-tolylphosphine, or tricyclohexylphosphine, and the like, a base, such as, sodium bicarbonate, triethylamine, or diisopropylethylamine, in a noninterfering solvent, such as, acetonitrile, triethylamine, or toluene at a temperature about 25 to 200° C. to form (19).

Examples of other suitable solvents include tetrahydrofuran, benzene, dimethylsulfoxide, or dimethylformamide.

Examples of other suitable palladium catalysts include Pd(PPh₃)Cl₂, Pd(OCOCF₃)₂, [(CH₃C₆H₄)₃P]₂PdCl₂, [(CH₃CH₂)₃P]₂PdCl₂, [(C₆H₁₁)₃P]₂PdCl₂, and [(C₆H₅)₃P]₂PdBr₂.

Examples of other suitable phosphines include triisopropylphosphine, triethylphosphine, tricyclopentylphosphine, 1,2-bis(diphenylphosphino)ethane, 1,3-bis(diphenylphosphino)propane, and 1,4-bis(diphenylphosphino)butane.

Examples of other suitable bases include tripropyl amine, 2,2,6,6-tetramethylpiperidine, 1,5-diazabicyclo[2.2.2]octane (DABCO), 1,8-diazabicyclo[5.4.0]undec-7-ene (DBU), 1,5-diazabicyclo[4.3.0]non-5-ene, (DBN) sodium carbonate, potassium carbonate, and potassium bicarbonate.

The cyclization of the instant process is preferably carried out with palladium(II)acetate as catalyst in the presence of either triphenylphosphine, tri-o-tolylphosphine, 1,3-bis(diphenylphosphino)propane, or tricyclohexylphosphine in acetonitrile as solvent and triethylamine as base at a temperature about 50 to 150° C. The reaction is substantially complete in about 1 hour to 14 days.

Alternatively, a preferred process for cyclization consists of the reaction of intermediate (26) with a palladacycle catalyst such as trans-di(μ-acetato)-bis[o-(di-o-tolylphosphino)benzyl]dipalladium (II) in a solvent such as dimethylacetamide (DMAC) at 120-140° C. in the presence of a base such as sodium acetate.

Intermediate (19) may be alkylated with an alkylating agent XCH₂R₄, where X is halo in the presence of a base to form (20). Suitable bases include potassium carbonate, sodium carbonate, lithium carbonate, cesium carbonate, sodium bicarbonate, potassium bicarbonate, potassium hydroxide, sodium hydroxide, sodium hydride, potassium hydride, lithium hydride, and Triton B (N-benzyltrimethylammonium hydroxide).

The reaction may or may not be carried out in the presence of a crown ether. Potassium carbonate and Triton B are preferred. The amount of alkylating agent is not critical, however, the reaction is best accomplished using an excess of alkyl halide relative to the starting material.

A catalytic amount of an iodide, such as sodium iodide or lithium iodide may or may not be added to the reaction mixture. The reaction is preferably carried out in an organic solvent, such as, acetone, dimethylformamide, dimethylsulfoxide, or acetonitrile. Other suitable solvents include tetrahydrofuran, methyl ethyl ketone, and t-butyl methyl ether.

The reaction is conducted at temperatures from about −10 to 100° C. preferably at ambient temperature, and is substantially complete in about 1 to 48 hours depending on conditions. Optionally, a phase transfer reagent such as tetrabutylammonium bromide or tetrabutylammonium chloride may be employed.

Intermediate (20) May by dehydrogenated by oxidation with 2,3-dichloro-5,6-dicyano-1,4-benzoquinone in a noninterfering solvent to form (21).

Suitable solvents include methylene chloride, chloroform, carbon tetrachloride, diethyl ether, methyl ethyl ketone, and t-butyl methyl ether. Toluene, benzene, dioxane, and tetrahydrofuran are preferred solvents. The reaction is carried out at a temperature about 0 to 120° C. Temperatures from 50 to 120° C. are preferred. The reaction is substantially complete in about 1 to 48 hours depending on conditions.

Intermediate (21) may be aminated with ammonia in the presence of a noninterfering solvent to form a(22). Ammonia may be in the form of ammonia gas or an ammonium salt, such as ammonium hydroxide, ammonium acetate, ammonium trifluoroacetate, ammonium chloride, and the like. Suitable solvents include ethanol, methanol, propanol, butanol, tetrahydrofuran, dioxane, and water. A mixture of concentrated aqueous ammonium hydroxide and tetrahydrofuran or methanol is preferred for the instant process. The reaction is carried out at a temperature about 20 to 100° C. Temperatures from 50 to 60° C. are preferred. The reaction is substantially complete in about 1 to 48 hours depending on conditions.

Alkylation of (22) is achieved by treatment with an alkylating agent of the formula XCH₂R⁹ where X is halo and R⁷⁰ is —CO₂R⁷¹, —SO₃R⁷¹, —P(O)(OR⁷¹)₂, or —P(O)(OR⁷¹)H, where R⁷¹ is an acid protecting group or a prodrug function, in the presence of a base in a noninterfering solvent to form (23). Methyl bromoacetate and t-butyl bromoacetate are the preferred alkylating agents.

Suitable bases include potassium carbonate, sodium carbonate, lithium carbonate, cesium carbonate, sodium bicarbonate, potassium bicarbonate, potassium hydroxide, sodium hydroxide, sodium hydride, potassium hydride, lithium hydride, and Triton B (N-benzyltrimethylammonium hydroxide). The reaction may or may not be carried out in the presence of a crown ether. Cesium carbonate and Triton B are preferred.

The amount of alkylating agent is not critical, however, the reaction is best accomplished using an excess of alkyl halide relative to the starting material. The reaction is preferably carried out in an organic solvent, such as, acetone, dimethylformamide, dimethylsulfoxide, or acetonitrile. Other suitable solvents include tetrahydrofuran, methyl ethyl ketone, and t-butyl methyl ether.

The reaction is conducted at temperatures from about −10 to 100° C. preferably at ambient temperature, and is substantially complete in about 1 to 48 hours depending on conditions. Optionally, a phase transfer reagent such as tetrabutylammonium bromide or tetrabutylammonium chloride may be employed.

Intermediate (23) may be optionally hydrolyzed with a base or acid to form desired product (24) and optionally salified.

Hydrolysis of (23) is achieved using a base such as sodium hydroxide, potassium hydroxide, lithium hydroxide, aqueous potassium carbonate, aqueous sodium carbonate, aqueous lithium carbonate, aqueous potassium bicarbonate, aqueous sodium bicarbonate, aqueous lithium bicarbonate, preferably sodium hydroxide and a lower alcohol solvent, such as, methanol, ethanol, isopropanol, and the like. Other suitable solvents include acetone, tetrahydrofuran, and dioxane.

Alternatively, the acid protecting group may be removed by organic and inorganic acids, such as trifluoroacetic acid and hydrochloric acid with or without a noninterferring solvent. Suitable solvents include methylene chloride, tetrahydrofuran, dioxane, and acetone. The t-butyl esters are preferably removed by neat trifluoroacetic acid.

The reaction is conducted at temperatures from about −10 to 100° C. preferably at ambient temperature, and is substantially complete in about 1 to 48 hours depending on conditions.

The starting material (16) is prepared by esterifying compound (15) with a alkyl halide=XPG; where X is halo and PG is an acid protecting group, in the presence of a base, preferably potassium carbonate or sodium cabonate, in a noninterferring solvent, preferably dimethylformamide or dimethylsulfoxide. The preferred alkyl halide is methyl iodide. The reaction is conducted at temperatures from about 0 to 100° C. preferably at ambient temperature, and is substantially complete in about 1 to 48 hours depending on conditions.

Alternatively the starting material (16) may be prepared by condensation with an alcohol HOPG, where PG is an acid protecting group, in the presence of a dehydrating catalyst such as, dicyclohexylcarbodiimide (DCC) or carbonyl diimidazole.

In addition, U.S. Pat. No. 4,885,338 and Jpn. Kokai Tokkyo Koho 05286912, Nov 1993 Hesei teach a method for preparing 2-fluoro-5-methoxyaniline derivatives.

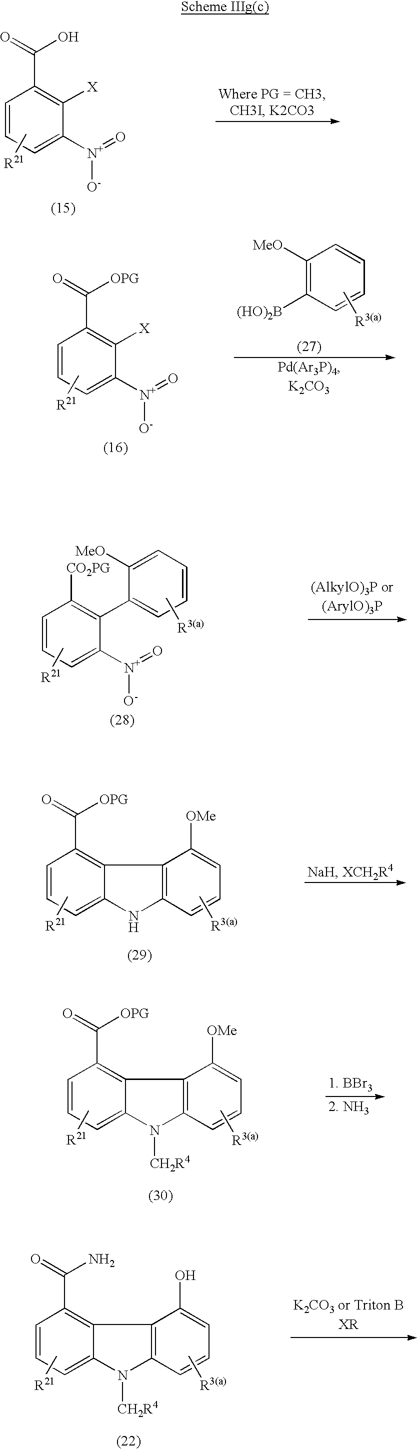

R is as defined in Scheme IIIg(b),

R^{3(a)} is as defined in Scheme Ig(a), above; and

X is halo.

Benzoic acid derivatives (16) (X=Cl, Br, or I) and boronic acid derivative (27) (either commercially available or readily prepared by known techniques from commercially available starting materials) are condensed under the general procedure of Miyaura, et al., (Ref 8a) or Trecourt, et al., (Ref 8b) in the presence of a palladium catalyst, such as Pd(Ph₃P)₄, a base, such as sodium bicarbonate, in an inert s6lvent, such as THF, toluene or ethanol, to afford compound (28).

Compound (28) is converted to the carbazole product (29) by treatment with a trialkyl or triaryl phosphite or phosphine, such as, triethylphosphite or triphenyl phosphine, according to the general procedure of Cadogan, et al. (Ref 6).

Compound (29) is N-alkylated with an appropriately substituted alkyl or aryl halide XCH₂R⁴ in the presence of a base, such as sodium hydride or potassium carbonate, in a noninterfering solvent, such as toluene, dimethylformamide, or dimethylsulfoxide to afford carbazole (30).

Compound (30) is converted to the corresponding amide (22) by treatment with boron tribromide or sodium thioethoxide, followed by ammonia or an ammonium salt, such as ammonium acetate, in an inert solvent, such as water or alcohol, or with methylchloroaluminum amide in an inert solvent, such as toluene, at a temperature between 0 to 110° C.

When R^{3(a)} is substituted at the 8-position with chloro, de-alkylation of (30) with boron tribromide results in de-benzylation of the nitrogen as described above. Alkylation may be readily accomplished in a two step process. First, an O-alkylation by treatment with a haloalkyl acetate such as methyl bromo acetate using sodium hydride in tetrahydrofuran, followed by N-alkylation using for example a base such as sodium hydride and an appropriately substituted alkyl or aryl halide in dimethoxy formamide. Compound (22) can be converted to product carbazole product (24) as described previously in Scheme IIIg(b) above.

Conversion to the desired prodrug may be accomplished by techniques known to the skilled artisan, such as for example, by treatment with a primary or secondary halide to make an ester prodrug.

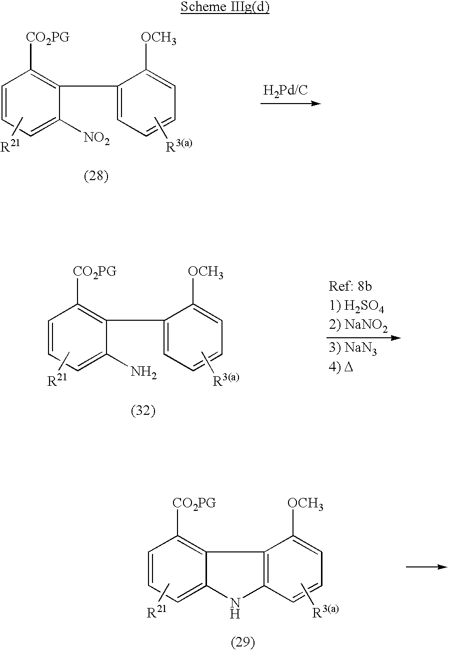

Alternatively, reduction of the nitro group of compound (28) with a reducing agent, such as hydrogen in the presence of palladium on carbon, in a noninterfering solvent, such as ethanol, at 1 to 60 atmospheres, at a temperature of 0 to 60° C. affords the corresponding aniline (32). Compound (32) is converted to the carbazole (29) according to the general procedure described by Trecourt, et al. (Ref 8b). The aniline is treated with sulfuric acid and sodium nitrite, followed by sodium azide to form an intermediate azide which is cyclized to carbazole (29) by heating in an inert sovent, such as toluene. Compound (29) is converted to carbazole product (24) as described previously in Schemes IIIg(b) and IIIg(c).

### REFERENCES

8) a. N. Miyaura, et al., Synth. Commun. 11, 513 (1981) b. F. Trecourt, et al., Tetrahedron, 51, 11743 6)

6) J. Cadogan et al., J. Chem. Soc., 4831 (1965)

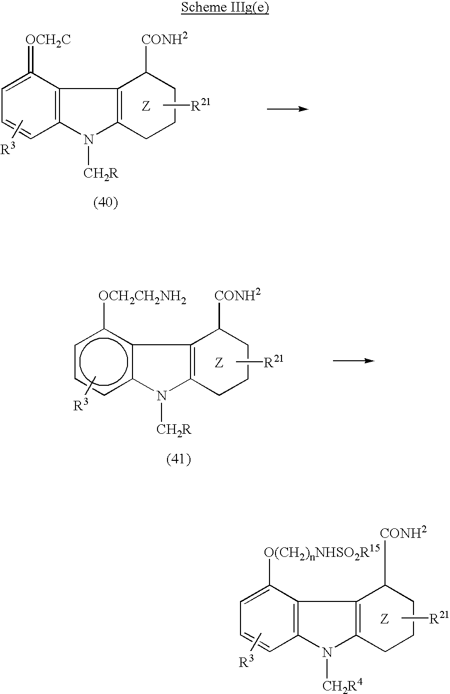

In an aprotic solvent, preferably tetrahydrofuran, reduction of (40) is achieved using a reducing agent such as aluminum trihydride. Preferably, the reaction is conducted under inert atmosphere such as nitrogen, at room temperature.

Sulfonylation may be achieved with an appropriate acylating agent in the presence of an acid scavenger such as triethyl amine.

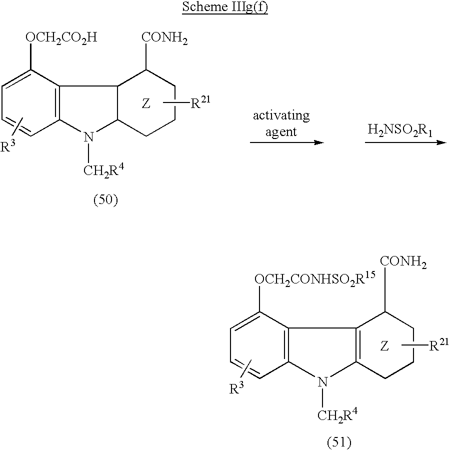

In a two-step, one-pot process, intermediate (50), prepared as described in Scheme I(a) above, is first activated with an activating agent such as carbonyl diimidazole. The reaction is preferably run in an aprotic polar or non-polar solvent such as tetrahydrofuran. Acylation with the activated intermediate is accomplished by reacting with H₂NSOR¹⁵ in the presence of a base, preferably diazabicycloundecene.

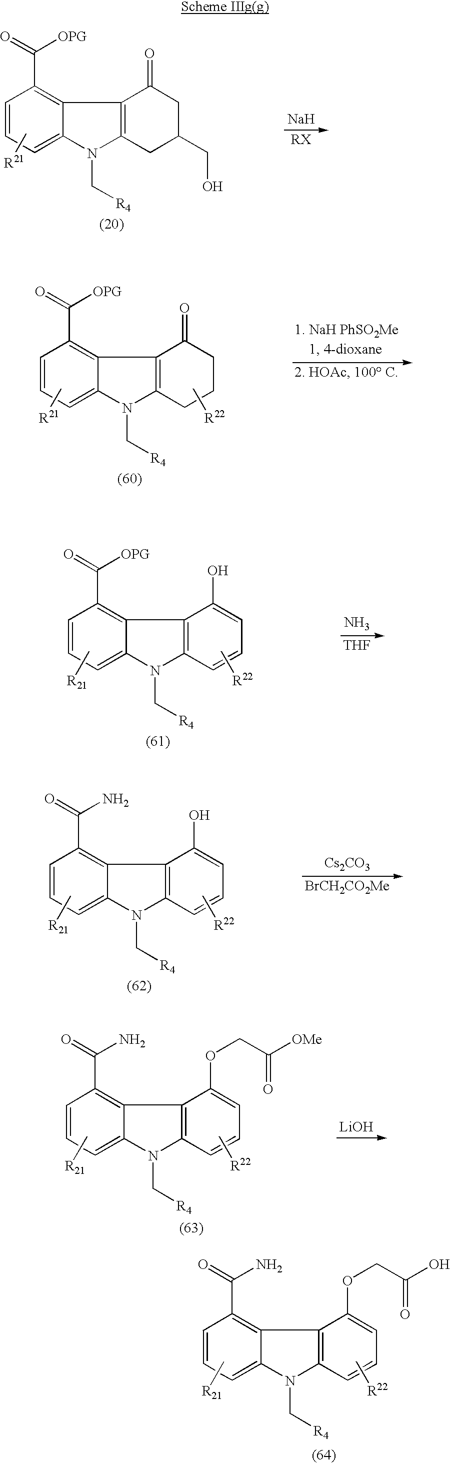

PG is an acid protecting group;

R²² is (C₁-C₆)alkoxy (C₁-C₆)alkyl is (C₁-C₆)alkoxy (C₁-C₆)alkenyl

Starting material (20) is O-alkylated with an alkyl halide or alkenyl halide, using a base such as NaH, in an aprotic polar solvent preferably anhydrous DMF, at ambient temperature under a nitrogen atmosphere. The process of aromatization from a cyclohexenone functionality to a phenol functionality can be performed by treating the tetrahydrocabazole intermediate (60) with a base such as NaH in the presence of methyl benzenesulfinate in an anhydrous solvent, such as 1,4-dioxane or DMF, to form the ketosulfoxide derivative. Upon heating at about 100° C. for 1-2 hours, the ketosulfoxide derivative (60) is converted to the phenol derivative (61). Conversion of the ester (61) to the amide (62) can be achieved by treating a solution of (61) in an aprotic polar solvent such as tetrahydrofuran with ammonia gas. Phenolic O-alkylation of (62) with, for example, methyl bromoacetate can be carried out in anhydrous DMF at ambient temperature using Cs₂CO₃ or K₂CO₃ as a base to form (63). Desired product (64) can be derived from the basic hydrolysis of ester (63) using LiOH or NaOH as a base in an H₂O/CH₃OH/THF solution at 50° C. for 1-2 hours.

When R²² is —(C₁-C₆)alkoxy(C₁-C₆)alkenyl, hydrogenation of the double bond can be performed by treating (63) in THF using PtO₂ as a catalysis under a hydrogen atmosphere. Desired product can then be derived as described above in Scheme III(g) from the basic hydrolysis of ester (63) using LiOH or NaOH as a base in an H₂O/CH₃OH/THF solution at 50° C. for 1-2 hours.

Compounds of formula le where the A ring is phenyl and the heteroatom in Z is sulfur, oxygen or nitrogen can be prepared as described in Schemes IV(a)-(f), below.

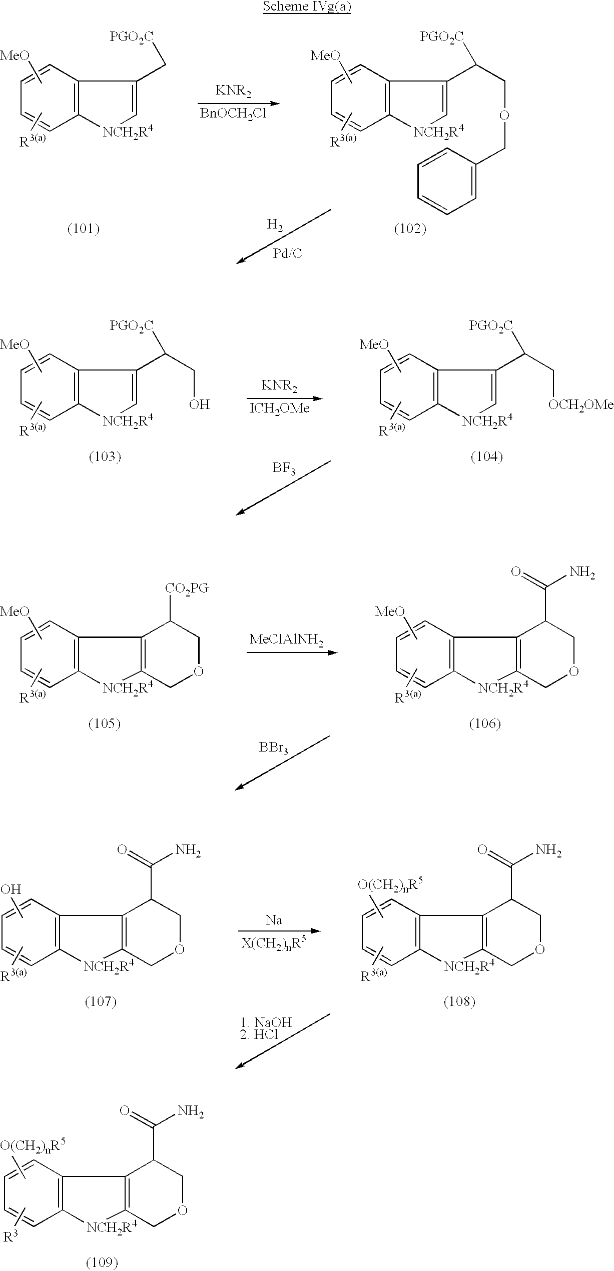

PG is an acid protecting group.

X is halo.

R³ (a) is H, —O(C₁-C₄)alkyl, halo, —(C₁-C₆)alkyl, phenyl, —(C₁-C₄)alkylphenyl; phenyl substituted with —(C₁-C₆)alkyl, halo or —CF³; —CH₂OSi(C₁-C₆)alkyl, furyl, thiophenyl, —(C₁-C₆)hydroxyalkyl; or —(CH₂)ₙR⁸ where R⁸ is H, —NR⁹R¹⁰, —CN or phenyl where R⁹ and R¹⁰ are independently —(C₁-C₄)alkyl or -phenyl(C₁-C₄)alkyl and n is 1 to 8;

An indole-3-acetic ester (101), Ref 10, is alkylated by treatment with alkalai metal amide and benzyloxymethyl chloride to give (102) which is converted to the alcohol (103) by catalytic hydrogenation. The alcohol is alkylated to provide the formaldehyde acetal (104) which is cyclized by Lewis acid to produce the pyrano[3,4-b]indole (105). The ester is converted to the amide (106) by methylchloroaluminum amide, and then to the phenol (107) with boron tribromide. The phenol is O-alkylated to give (108) which is hydrolyzed to the acid (109).

10) Dillard, R. et al., J, Med Chem. Vol 39, No. 26, 5119-5136.

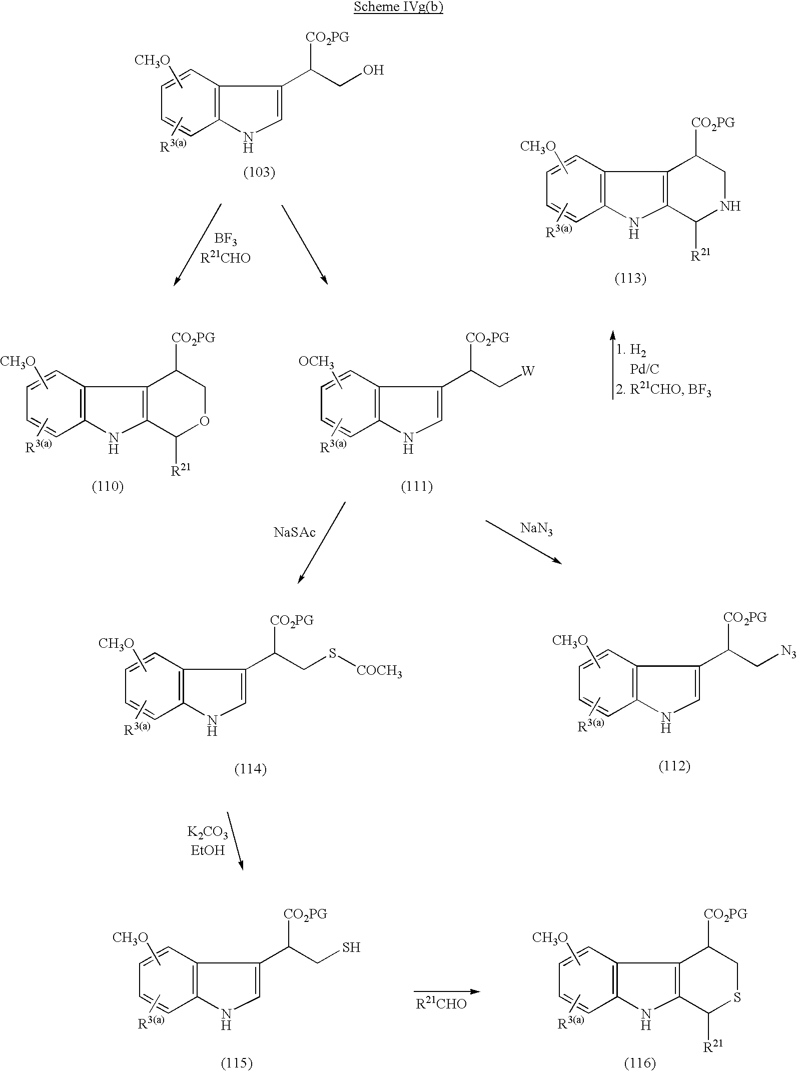

PG is an acid protecting group

W is halo, alkyl or aryl sulfonyl

R³ (a) is H, —O (C₁-C₄)alkyl, halo, —(C₁-C₆)alkyl, phenyl, —(C₁-C₄)alkylphenyl; phenyl substituted with —(C₁-C₆)alkyl, halo or —CF³; —CH₂OSi(C₁-C₆)alkyl, furyl, thiophenyl, —(C₁-C₆)hydroxyalkyl; or —(CH₂)ₙR⁸ where R⁸ is H, —NR⁹R¹⁰, —CN or phenyl where R⁹ and R¹⁰ are independently —(C₁-C₄)alkyl or -phenyl(C₁-C₄)alkyl and n is 1 to 8;

Reaction of this alcohol (103) with aldehyde and acid produces the pyranoindole (110).

Conversion of the hydroxyl function of (103) to a halide or sulfate functionality is achieved by treatment with triphenylphosphine and CH₃X (where X is a halogen) to make compounds of formula (111) where X is a halide; or by treatment with triethylamine and methanesulfonyl chloride to make the sulfonate. Displacement with the sodium salt of thiol acetic acid gives (114) which in turn is hydrolyzed by base to the thiol (115) which is reacted with an appropriately substituted aldehyde and acid to produce the thiopyranoindoles (116).

Intermediate (111) may also be reacted with sodium azide to give the azido derivative (112) which is reduced by hydrogen catalytically to give the amine which is converted to the carboline (113) with aldehyde and acid.

Intermediates (113), (110) and (116) may be N-alkylated, using sodium hydride and an appropriately substituted alkylhalide XCH₂R⁴.

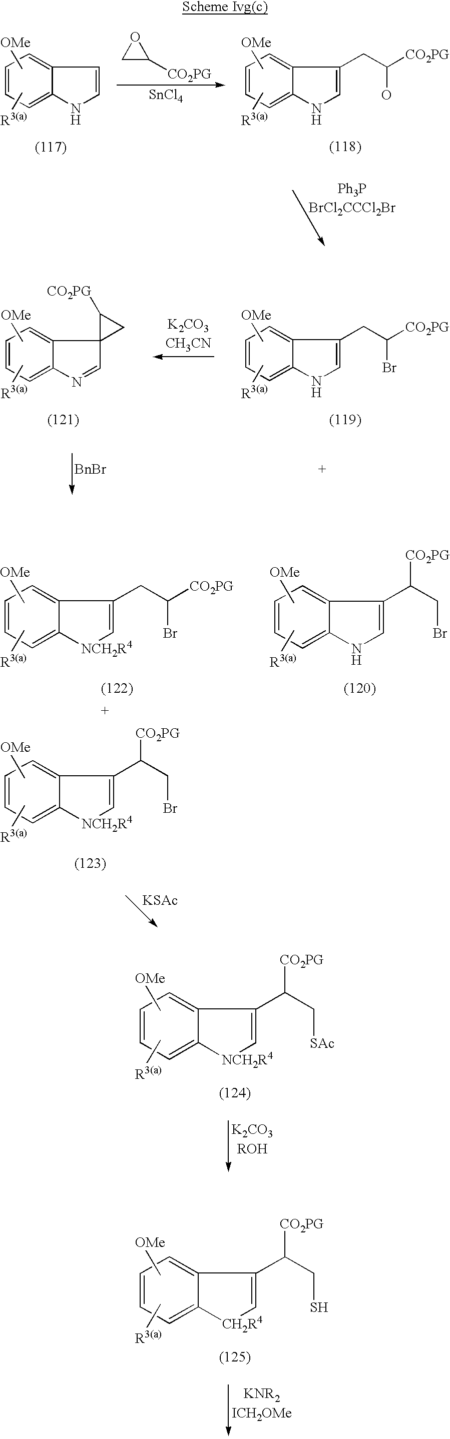

4-Methoxyindole (117) is converted to the indole acetic acid derivative (118) by alkylation with an epoxy propionate. Treatment of (118) with a brominating reagent affords the mixture of bromo isomers (119) and (120) which give the spiro compound (121) upon basic treatment. Heating (121) with benzyl bromide provides a mixture of the isomeric bromo compounds (122) and (123) which react with potassium thioacetate to give a mixture of isomers from which (124) may be separated. Solvolysis of the thioester produces the thiol (125) which is alkylated to give (126). Lewis acids convert (126) to the thiopyrano[3,4-b]indole (127). The ester function is converted to amide using methylchloroaluminum amide, the methyl ether cleaved by boron tribromide, and the product phenol O-alkylated with bromoacetic ester to give (130) which is hydrolyzed to (131).

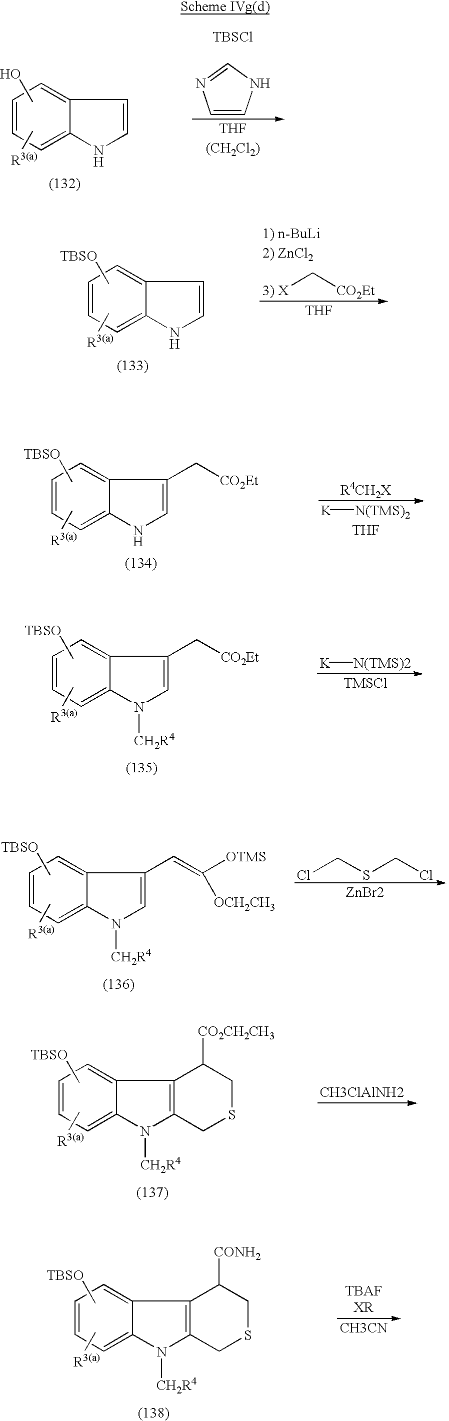

X is halo

R^{3(a)} is as defined in Scheme I(a) above; and

R is —(CH₂)mR⁵.

Protection of the oxygen by treatment of (132) with tert-butyldimethylsilyl chloride and imidazole in an aprotic polar solvent such as tetrahydrofuran or methylene chloride accomplishes (133).

Alkylation at the 3-position of the indole (133) is achieved by treatment with n-butyllithum then zinc chloride at temperatures starting at about 10° C. and warming to room temperature, followed by reaction with an appropriate haloalkyl ester such as methyl or ethyl bromoacetate. The reaction is preferably conducted at room temperature in an appropriate aprotic polar solvent such as tetrahydrofuran.

Alkylation of the indole-nitrogen can then be achieved by reacting (134) with a suitable alkyl halide in the presence of potassium bis(trimethylsilyl)amide to prepare (135).

The ester functionality of (135) is converted to a trimethylsilylketene acetal (136) by treatment with potassium bis(trimethylsilyl)amide and trimethylsilyl chloride. Treatment of the ketene acetal (136) with bis(chloromethyl)sulfide and zinc bromide in methylene chloride affords the cyclized product (137). Conversion to amide (138) can be accomplished by a Weinreb reaction with methylchloroaluminum amide. Removal of the oxygen protecting group with a fluoride source, such as tetrabutylammonium fluoride (TBAF), and concommitant reaction of the resulting anion with, for example, ethyl bromoacetate yields the ester (139). Deprotection of the ester yields the desired acid (140).

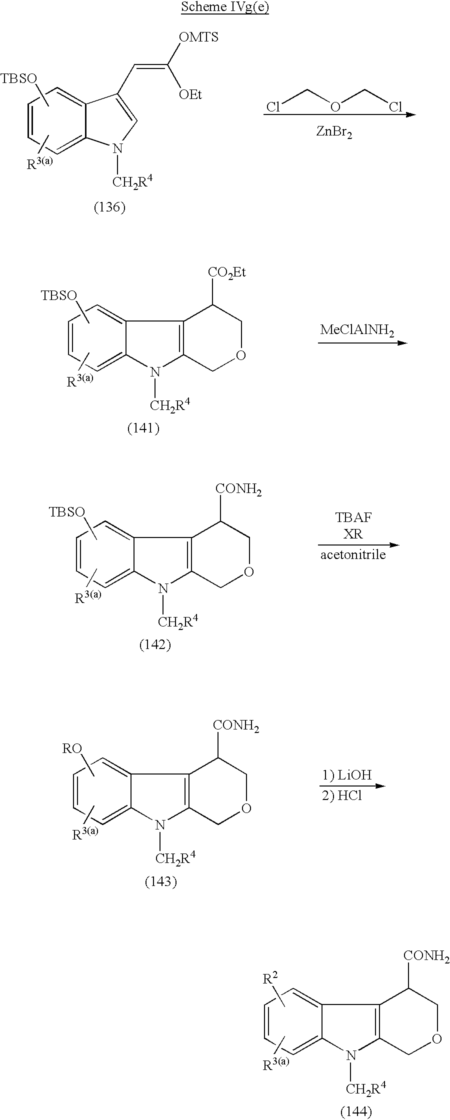

R^{3(a)} is as described in Scheme I(a) and

R is as described in Scheme IV(d).

Treatment of the ketene acetal (136) with bis(chloromethyl)ether and zinc bromide in methylene chloride affords the cyclized product (141). Conversion to amide (142) can be accomplished by a Weinreb reaction with methylchloroaluminum amide. Removal of the oxygen protecting group with a fluoride source, such as tetrabutylammonium fluoride, and concommitant reaction of the resulting anion with ethyl bromoacetate yields the ester (143). Deprotection of the ester yields the desired acid (144).

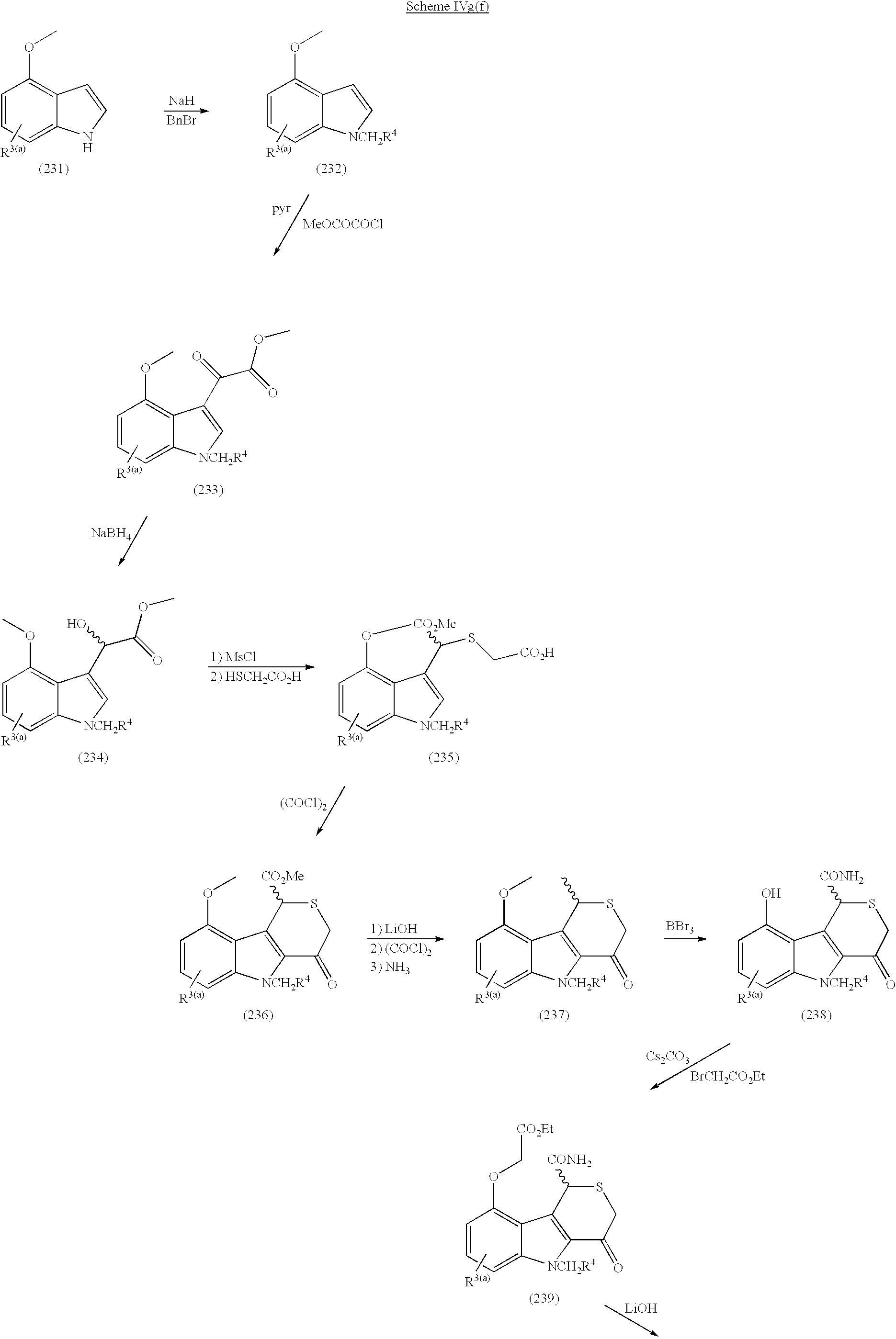

N-alkylation of commercially available 4-methoxy indole (231) under basic conditions using an alkyl halide affords the N-alkyl indole (232). Acylation with a suitable acid chloride provides the glyoxalate ester product (233) which can be reduced with a variety of hydride reducing agents to give intermediate alcohols (234). Conversion of the alcohol to a suitable leaving group and displacement with sulfur nucleophiles affords the thioether product (235). Conversion to the acid chloride and spontaneous cyclization affords the thioketone product (236). Cleavage of the ester can be effected under basic conditions to give the correponding acid which upon formation of the acid chloride and reaction with an appropriate amine gives the amide product (237). Cleavage of the methyl ether gives the phenol (238) which can be alkylated under basic conditions using alkyl halides to give the O-alkylated product (239). Cleavage of the ester under basic conditions gives the desired product (240). Alternatively, reduction of the benzylic ketone with a hydride reducing agent and subsequent deoxygenation of the resulting alcohol gives the deoxygenated product (244). Cleavage of the oxyacetic ester proceeds under basic conditions to give the desired oxyacetic acid (242).

Compounds where Z is an aromatic or heterocyclic ring containing nitrogen can be prepared as described in Schemes Vg(a)-(e), below.

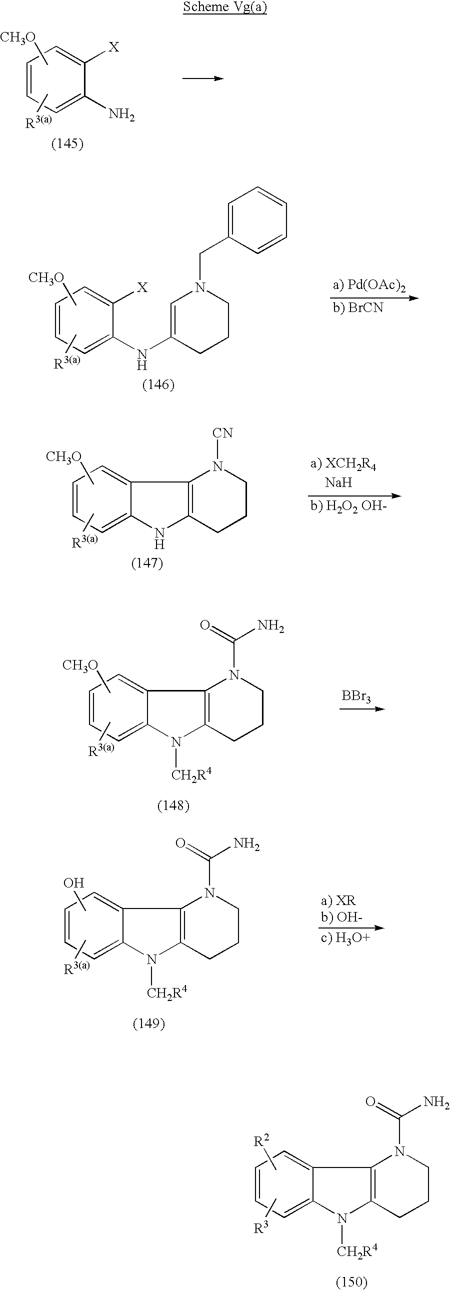

Substituted haloaniline (145) is condensed with N-benzyl-3-piperidone to provide enamine (146). Ring closure is effected by treatment of (146) with palladium (II) acetate and the resultant product is converted to (147) by treatment with cyanogen bromide. Alkylation of (147) is accomplished by treatment with the appropriate alkyl bromide using sodium hydride as base. Hydrolysis of this N-alkylated product with basic hydrogen peroxide under standard conditions provides (148). Demethylation of (148) is carried out by treatment with boron tribromide in methylene chloride. The resulting phenol (149) is converted by the standard sequence of O-alkylation with methyl bromoacetate in the presence of a base, hydrolysis with hydroxide to provide the intermediate salt which is then protonated in aqueous acid to provide desired δ-carboline (150).

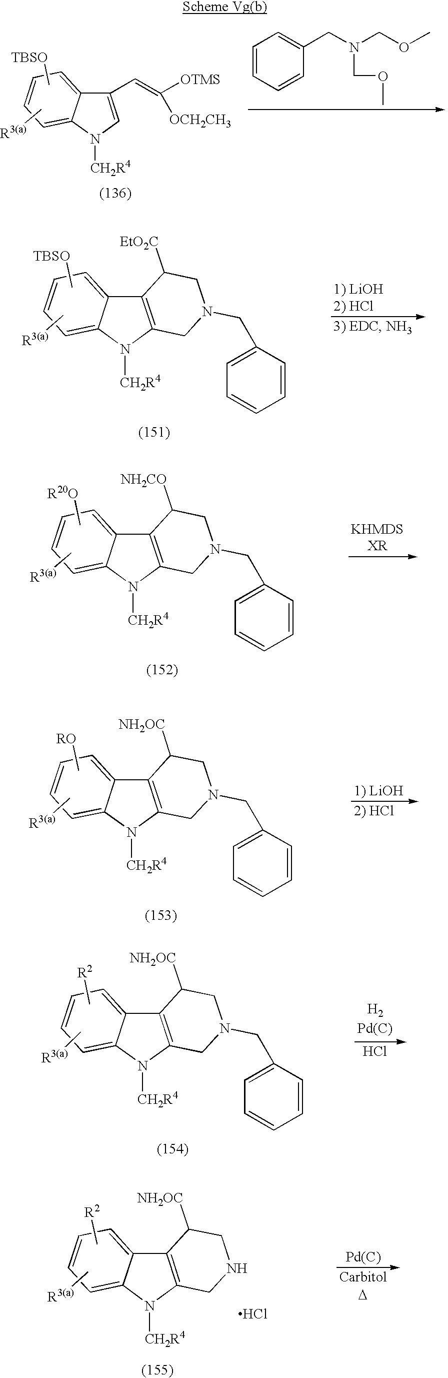

X is halo,

R is as defined in Scheme IV(d), and

R^{3(a)} is as defined in Scheme I(a).

Ketene acetal (136), prepared as described in Scheme IV(d), is reacted with benzyl bis(methoxyymethyl)amine in the presence of zinc chloride to give the tetrahydro-beta-carboline (151).

Treatment of (151) with lithium hydroxide, neutralization with hydrochloric acid and subsequent treatment with 1-(3-dimethylaminopropyl)-3-ethylcarbodiimide hydrochloride and ammonia provides the desilyated amide (152) where R²⁰ is hydrogen, which can be alkylated with, for example, ethylbromoacetate to give ester (153).

Alternatively, treatment of (115) with the appropriate Weinreb reagent provides amide (152) (R²⁰ is t-butyldimethylsilyl) which is desilylated with tetra-n-butylammonium fluoride and alkylated with, for example, ethyl bromoacetate to give ester (153). Lithium hydroxide-mediated hydrolysis gives acid (154), which may be hydrogenated over an appropriate catalyst in the presence of hydrochloride acid to give the tetrahydro-beta-carboline as the hydrochloride salt (155). Compound (155) may in turn be aromatized by refluxing in carbitol with palladium on carbon to provide beta-carboline (156).

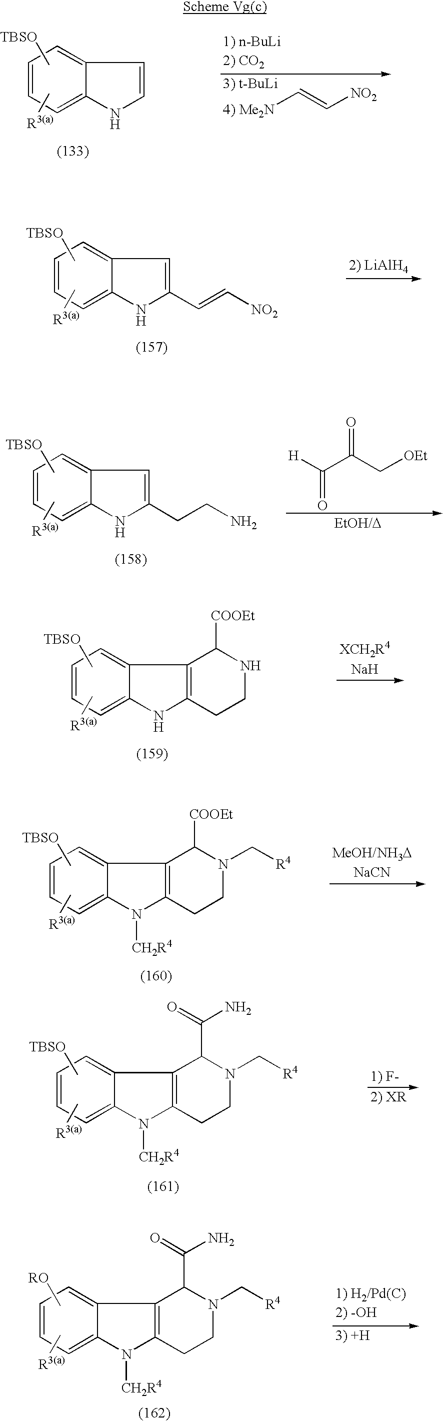

X is halo,

R is as defined in Scheme IV(d); and

R^{3(a)} is as defined in Scheme I(a).

In a one-pot reaction, indole (133) is successively treated with one equivalent n-butyllithium, carbon dioxide gas, one equivalent of t-butyllithium, and 1-dimethylamino-2-nitroethene to give (157). Nitroalkene (157) is reduced with lithium aluminum hydride to amine (158), which is cyclized with methyl glyoxylate (Ref. 9) in refluxing ethanol to give tetrahydrocarboline (159). Alkylation of both nitrogens of (159) leads to intermediate (160), which is treated with the appropriate Weinreb reagent to provide amide (161). Fluoride-assisted desilylation and alkylation with, for example, ethyl iodoacetate gives ester (162), which may be hydrogenated over a suitable catalyst and base-hydrolyzed to give acid (163). Aromatization of (163) to carboline (164) is achieved by refluxing in carbitol in the presence of palladium-on-carbon.

### REFERENCE 9

Kelley, T. R.; Schmidt, T. E.; Haggerty, J. G. A convenient preparation of methyl and ethyl glyoxylate, Synthesis, 1972, 544-5.

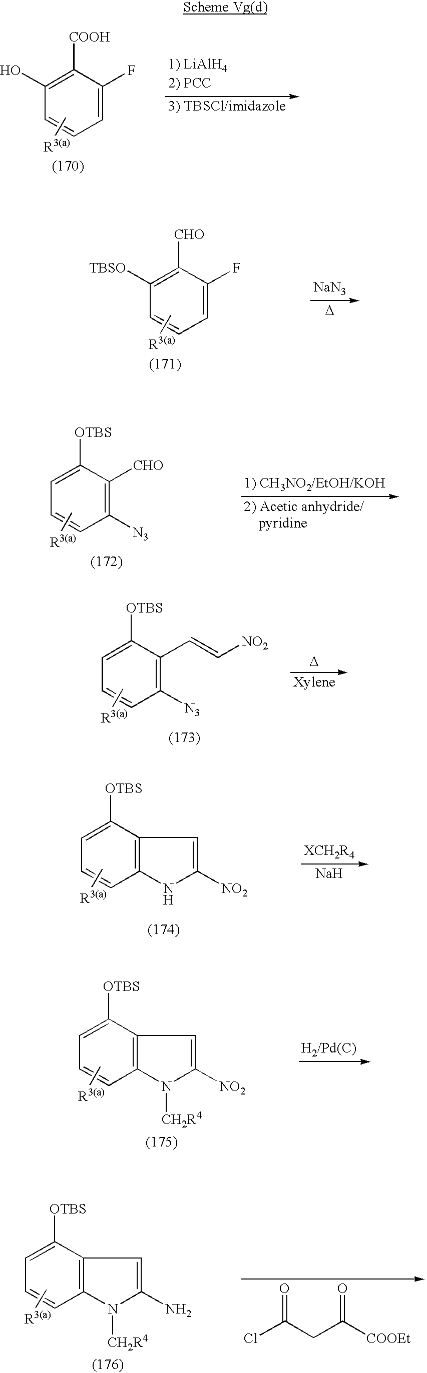

The commercially available acid (170) is reduced with lithium aluminum hydride, oxidized with pyridinium chlorochromate, and silylated with t-butyldimethylsilyl chloride to give (171). Treatment with sodium azide provides azide (172), which is reacted with nitromethane and potassium hydroxide in ethanol, followed by treatment with acetic anhydride and pyridine to give nitroolefin (173). Heating in xylene induces cyclization to produce indole (174). Alkylation with, for example, benzyl iodide and sodium hydride gives (175), which is hydrogenated in the presence of palladium-on-carbon to give amine (176). Acylation with the acid chloride of commercially available oxalacetic acid monoethyl ester gives (177), which is thermally cyclized to lactam (178). Selective reduction of the lactam carbonyl may be accomplished by treatment with NaBH₂S₃ to provide amine (179).

Protection of amine (179) with di-t-butyl dicarbonate and pyridine produces (180), which is converted via the appropriate Weinreb reagent to amide (181). Fluoride-assisted desilylation, alkylation, with, for example, ethyl iodoacetate and potassium carbonate, base hydrolysis, and acid hydrolysis produce the tetrahydro-alpha-carboline (182).

Alternatively, amine (179) may be aromatized by refluxing in carbitol or some other suitable high boiling solvent to give alpha-carboline (183), which is converted via the appropriate Weinreb reagent to amide (184). Fluoride-assisted desilylation, alkylation with ethyl iodoacetate and potassium carbonate, and base hydrolysis as described above provides alpha-carboline (185).

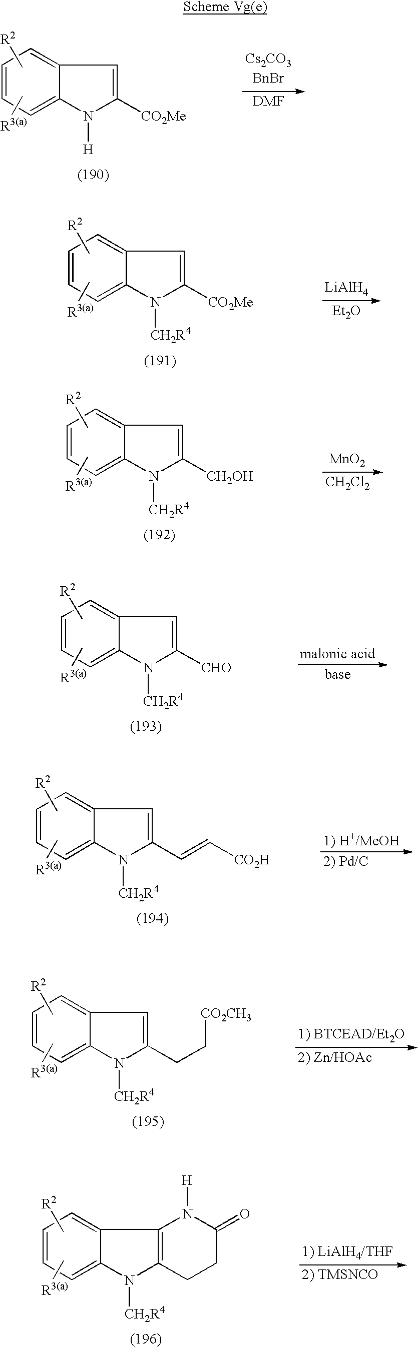

X is halo

R^{3(a)} is as defined above Scheme V(e) provides δ-carboline (198) by the indicated sequence of reactions. N-alkylation of 2-carboethoxyindole (190) followed by a standard two carbon homologation sequence provides 2-(3-propenoic acid)indoles (194). In this sequence, the condensation of aldehyde (193) with malonic acid utilized a mixture of pyridine and piperidine as the base. After methyl ester formation and hydrogenation (195), ring closure (196) was effected by treatment with bis(2,2,2-trichloroethyl)azodicarboxylate (BTCEAD) followed by zinc in acetic acid. Reduction of the cyclic amide with lithium aluminum hydride followed by treatment with trimethylsilylisocyanate provided the urea (197). Conversion to the desired d-carboline (198) was accomplished under the usual conditions of demethylation and subsequent alkylation and ester hydrolysis steps.

Reverse indoles, i.e., compounds where B is carbon and D is nitrogen can be prepared as described in Scheme VIg, below.

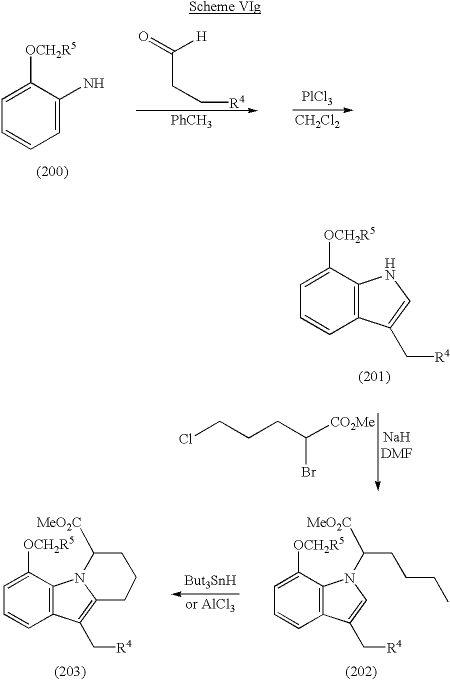

Aryl hydrazines (200) are condensed with substituted prpionaldehydes to form hydrazones which are cyclized to indoles (201) by treatment with phosphorous trichloride at room temperature (Ref 1). The indoles are N-alkylated on reaction with a base such as sodium hydride and an alph-bromo ester to give indoles (202) which are cyclized to tetrahydrocarbazoles (203) by Lewis acids (e.g., aluminum chloride) or by radical initiators (e.g., tributyltin hydride). Compounds (203) can be converted to carbazoles by, for example, refluxing in a solvent such as carbitol in the presence of Pd/C.

Compounds of formula I wherein A is pyridyl can be prepared as described in Schemes VIIg(a)-(b), below.

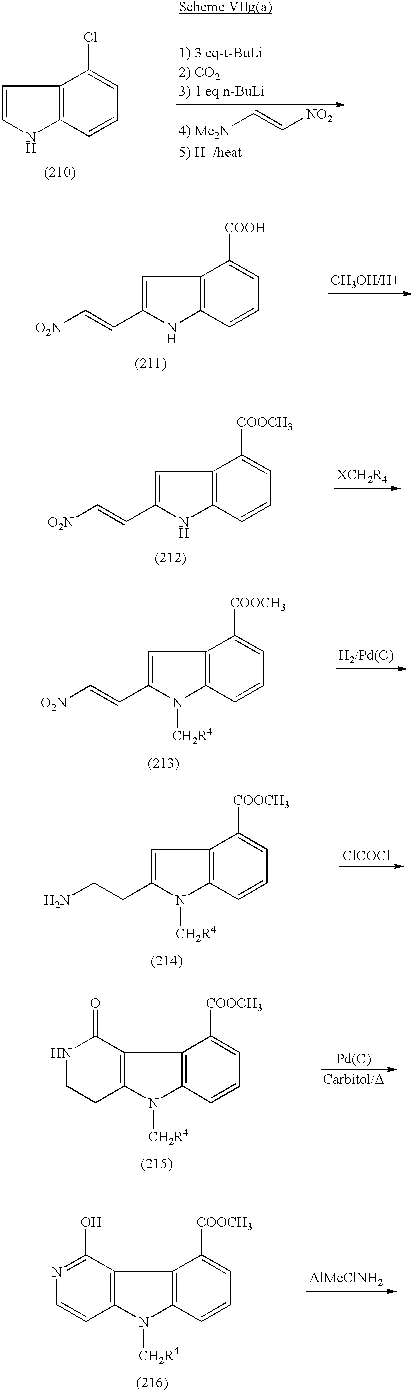

X is halo and

R is (CH₂)ₘR⁵.

Commercially available 4-chloroindole (210) is treated with 3 equivalents of t-butyllithium followed by carbon dioxide, 1 equivalent of n-butyllithium, 1-dimethylamino-2-nitroethene, and acid to provide carboxylic acid (211), which may be esterified to give (212). Alkylation at the 1-position followed by hydrogenation provides aminoethyl indole (214). Cyclization with phosgene to (215) followed by aromatization gives carboline (216). Treatment of (216) with the appropriate Weinreb reagent provides amide (217), which may be alkylated with, for example, ethyl bromoacetate and saponified with sodium hydroxide to give the carboline (218).

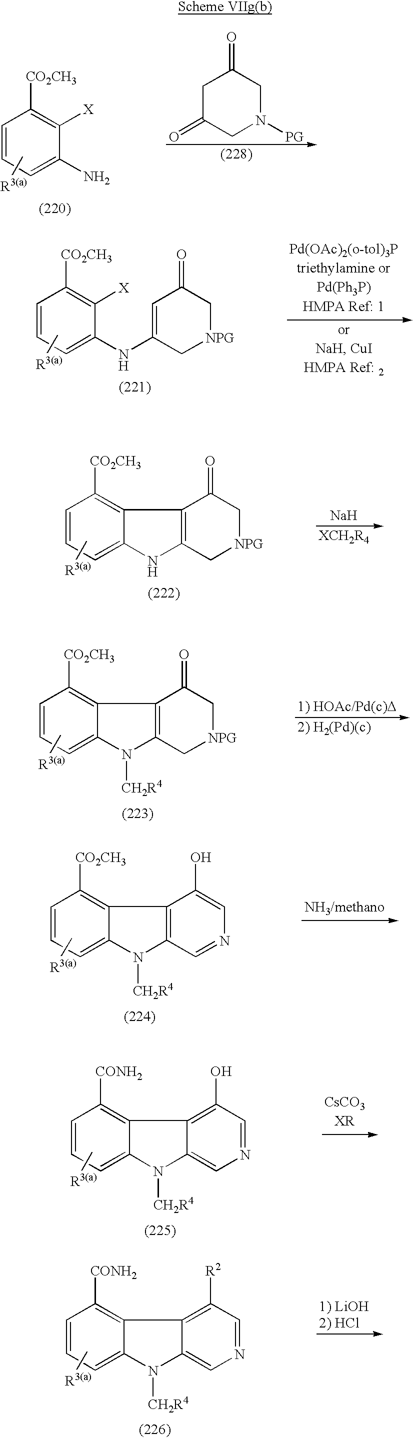

R3(a) is as defined in Scheme I(a),

X is halo, and

R is (CH₂)ₘR⁵.

The 1,3-dione structures (228) are either commercially available or readily prepared by known techniques from commercially available starting materials. Preparation of the aniline derivatives (220) (X=Cl, Br, or I) are accomplished by reducing an appropriately substituted benzoic acid derivative to the corresponding aniline by treatment with a reducing agent such as SnCl₂ in hydrochloric acid in an inert solvent such as ethanol or by hydrogenation using hydrogen gas and sulfided platinum or carbon or palladium on carbon. The amino group of (228) is protected with an appropriate protecting group, such as the, carboethoxyl, benzyl, CBZ (benzyloxycarbonyl) or BOC (tert-butoxycarbonyl) protecting group, and the like.

The dione (228) and aniline derivative (220) are condensed according to the general procedure of Chen, et al., (Ref 10) or Yang, et al., (Ref 11), with or without a noninterfering solvent, such as methanol, toluene, or methylene chloride, with or without an acid, such as p-toluenesulfonic acid or trifluoroacetic acid, with or without N-chlorosuccinimide and dimethyl sulfide, to afford the coupled product (221).

Compound (221) is cyclized under basic conditions with a copper (I) salt in an inert solvent according to the general procedure of Yang, et al., (Ref †8). The derivative (221) is treated with a base, such as sodium hydride, in an inert solvent, such as HMPA, at a temperature between 0 and 25° C. A copper (I) salt, such as copper (I) iodide, is added and the resultant mixture stirred at a temperature between 25 and 150° C. for 1 to 48 hours to afford compound (222).

Compound (221) may also be cyclized according to the general procedure of Chen, et al., (Ref 10). The derivative (221) is treated with a base, such as sodium bicarbonate, and a palladium catalyst, such as Pd(PPh₃)₄, in an inert solvent, such as HMPA, at a temperature between 25 and 150° C. to afford compound (222).

In a preferred method, intermediate (171) is treated with a transition metal catalyst, such as Pd(OAc)₂(O-tol)₃P in the presence of a base such as triethylamine using a cosolvent of DMF/acetonitrile to prepare (222).

Compound (222) is N-alkylated with an appropriately substituted benzyl halide in the presence of a base, such as sodium hydride or potassium carbonate, in a noninterfering solvent, such as dimethylformamide or dimethylsulfoxide to afford ketone (223). In a two step, one pot process(222) is aromatized by treatment with acetic acid and palladium on carbon in a noninterfering solvent, such as carbitol or cymene, followed by treatment with hydrogen gas and palladium on carbon to cleave the nitrogen protecting group and produce the phenolic derivative (224).

The ester (224) is converted to the corresponding amide (225) under standard conditions with ammonia (preferably) or an ammonium salt, such as ammonium acetate, in an inert solvent, such as water or alcohol, preferably methanol, or with MeClAlNH₂ in an inert solvent, such as toluene, at a temperature between 0 to 110° C. Alkylation of the phenolic oxygen of compound 38 with an appropriate haloester, such as methyl bromoacetate, in the presence of a base, such as cesium carbonate, potassium or sodium carbonate, in an inert solvent, such as dimethylformamide or dimethylsulfoxide affords the ester-amide (226). Other haloesters, such as ethyl bromoacetate, propyl bromoacetate, butyl bromoacetate, and the like can also be used to prepare the corresponding esters.

Saponification of compound (226), with lithium hydroxide in an inert solvent, such as methanol-water, affords (227). The intermediate and final products may isolated and purified by conventional techniques such as chromatography or recrystallization. Regioisomeric products and intermediates can be separated by standard methods, such as, recrystallization or chromatography.

### REFERENCES

10) L. -C. Chen et al., Synthesis 385 (1995)

11) S. -C. Yang et al., Heterocycles, 32, 2399 (1991)

h) Pyrazole sPLA₂ Inhibitors

The compositions and method of the invention may be prepared and practiced using pyrazole sPLA2 inhibitors, which are described (together with the method of making) in U.S. patent application Ser. No. 08/984,261, filed Dec. 3, 1997, the entire disclosure of which is incorporated herein by reference. Suitable pyrazole compounds are represented by formula (Ih)

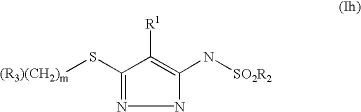

wherein:

R¹ is phenyl, isoquinolin-3-yl, pyrazinyl, pyridin-2-yl, pyridin-2-yl substituted at the 4-position with —(C₁-C₄)alkyl, (C₁-C₄)alkoxyl, —CN or —(CH₂)ₙCONH₂ where n is 0-2;

R² is phenyl; phenyl substituted with 1 to 3 substituents selected from the group consisting of —(C₁-C₄)alkyl, —CN, halo, —NO₂, CO₂(C₁-C₄)alkyl and —CF₃; naphthyl; thiophene or thiophene substituted with 1 to 3 halo groups;

R³ is hydrogen; phenyl; phenyl(C₂-C₆)alkenyl; pyridyl; naphthyl; quinolinyl; (C₁-C₄)alkylthiazolyl;

phenyl substituted with 1 to 2 substituents selected from the group consisting of —(C₁-C₄)alkyl, —CN, —CONH₂, —NO₂, —CF₃, halo, (C₁-C₄)alkoxy, CO₂(C₁-C₄)alkyl, phenoxy and SR⁴ where R⁴ is —(C₁-C₄)alkyl or halophenyl; phenyl substituted with one substituent selected from the group consisting of

—O(CH₂)ₚR⁵ where p is 1 to 3 and R⁵ is —CN, —CO₂H, —CONH₂, or tetrazolyl,

phenyl and

—OR⁶ where R⁶ is cyclopentyl, cyclohexenyl, or phenyl substituted with halo or (C₁-C₄)alkoxy;

or phenyl substituted with two substituents which, when taken together with the phenyl ring to which they are attached form a methylenedioxy ring; and

m is 1 to 5;

or a pharmaceutically acceptable salt thereof.

Particularly preferred are pyrazole type sPLA₂ inhibitors as follows:

A pyrazole compound of formula (I), supra, wherein:

R¹ is pyridine-2-yl or pyridine-2-yl substituted at the 4-position with —(C₁-C₄)alkyl, (C₁-C₄)alkoxy, —CN or —(CH₂)ₙCONH₂ where n is 0-2;

R² is phenyl substituted with 1 to 3 substituents selected from the group consisting of —(C₁-C₄)alkyl, —CN, halo, —NO₂, CO₂(C₁-C₄)alkyl and —CF₃; and

R³ is phenyl; phenyl(C₂-C₆)alkenyl; phenyl substituted with 1 or 2 substituents selected from the group consisting of —(C₁-C₄)alkyl, —CN, —CONH₂, —NO₂, —CF₃, halo, (C₁-C₄)alkoxy, CO₂(C₁-C₄)alkyl, phenoxy and SR₄ where R⁴ is —(C₁-C₄)alkyl or halo phenyl;

phenyl substituted with one substituent selected from the group consisting of —O(CH₂)ₚR⁵ where p is 1 to 3 and R⁵ is —CN, —CO₂H, —CONH₂ or tetrazolyl, phenyl and —OR⁶ where R⁶ is cyclopentyl, cyclohexenyl or phenyl substituted with halo or (C₁-C₄)alkoxy; or phenyl substituted with two substituents which when taken together with the phenyl ring to which they are attached form a methylenedioxy ring.

Specific suitable pyrazole type sPLA₂ inhibitors useful in the method of the invention are as follows: Compounds selected from the group consisting of 3-(2-chloro-6-methylphenylsulfonylamino) -4-(2-(4-acetamido)pyridyl)-5-(3-(4-fluorophenoxy)benzylthio)-(1H)-pyrazole and 3-(2,6-dichlorophenylsulfonylamino)-4-(2-(4-acetamido)pyridyl)-5-(3-(4-fluorophenoxy)benzylthio)-(1H)-pyrazole.

The pyrazole compounds of formula Ih are prepared as described in Scheme Ih below.

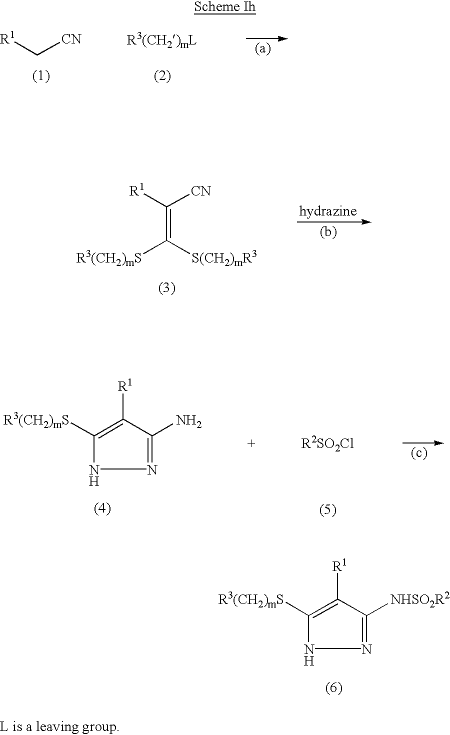

In an aprotic polar solvent, such as tetrahydrofuran, an acetonitrile compound (1) is deprotonated by treatment with an excess of a strong base, such as sodium hydride, preferably under an inert gas, such as nitrogen. The deprotonated intermediate is treated with carbon disulfide and then alkylated twice with an appropriately substituted alkyl halide (2) of the formula R³(CH₂)ₘL, where L is a leaving group, preferably bromine, and R³ and m are as defined above, to prepare intermediate compound (3). The reaction is conducted at ambient temperatures and is substantially complete in 1 to 24 hours.

Cyclization to form the amino substituted pyrazole (4) is achieved by reacting intermediate (3) with hydrazine at room temperature for from about 1 to 24 hours.

Selective sulfonylation of the amino group of intermediate (4) can be accomplished by treatment with a sulfonyl chloride (5) of the formula R²SO₂Cl, where R² is as defined above, to prepare product (6). The reaction is preferably conducted in a solvent, such as pyridine, at ambient temperature for a period of time of from 1 to 24 hours. Preparation of 2,6-dimethylphenylsulfonyl chloride can be accomplished as described in J. Org. Chem. 25, 1996 (1960). All other sulfonyl chlorides are commercially available.

i) Phenyl glyoxamide sPLA₂ inhibitors (and the method of making them) are described in U.S. patent application Ser. No. 08/979,446, filed Nov. 24, 1997 (titled, Phenyl Glyoxamides as sPLA₂ Inhibitors), the entire disclosure of which is incorporated herein by reference.

The compositions and method of the invention is for treatment of a mammal, including a human, afflicted with sepsis may be practiced using phenyl glyoxamide type sPLA₂ inhibitors described as follows:

A compound of the formula (Ii)

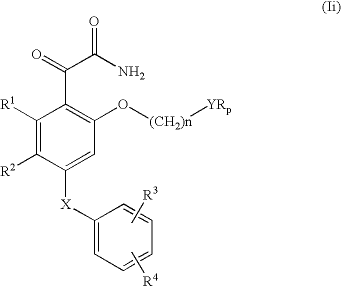

wherein:

X is —O— or —(CH₂)ₘ—, where m is 0 or 1;

Y is —CO₂—, —PO₃—, —SO₃—;

R is independently —H or —(C₁-C₄)alkyl;

R¹ and R² are each independently —H, halo or —(C₁-C₄)alkyl;

R³ and R⁴ are each independently —H, —(C₁-C₄)alkyl, (C₁-C₄)alkoxy, (C₁-C₄)alkylthio, halo, phenyl or phenyl substituted with halo;

n is 1-8; and

p is 1 when Y is —CO₂— or —SO₃— and 1 or 2 when Y is —PO₃—;

or a pharmaceutically acceptable salt thereof.

A specific suitable phenyl glyoxamide type sPLA₂ inhibitors is 2-(4-carboxybut-1-yl-oxy)-4-(3-phenylphenoxy)phenylglyoxamide.

Phenyl glyoxylamide compounds useful in the compositons and method of the invention are prepared as follows:

Compounds where R¹, R², R³ and R⁴ are H, and X, Y and n and p are as defined above can be prepared according to the following Scheme Ii.

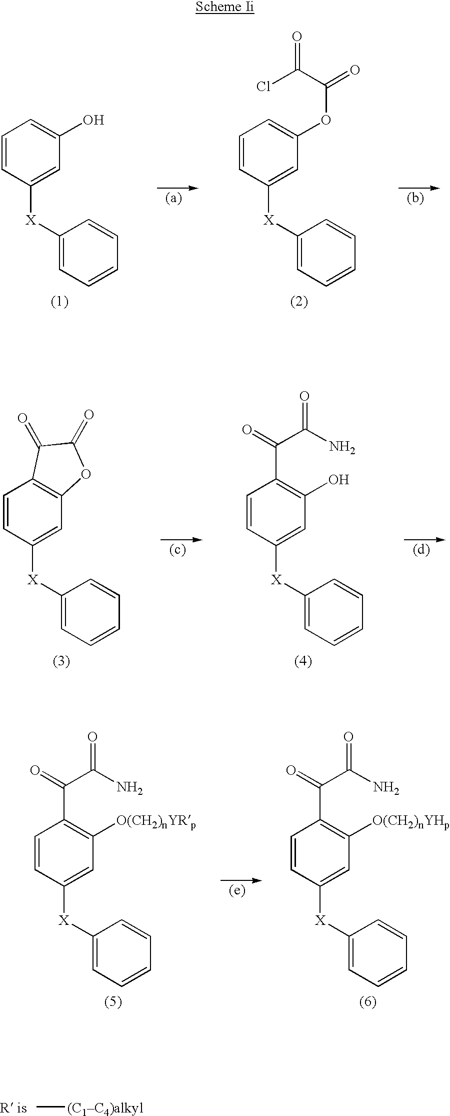

Reflux of (1) with oxalyl chloride in an alkyl halide solvent, such as chloroform, using 4-N,N′ dimethylamino pyridine as a catalyst achieves intermediate (2).

Under Friedel-Crafts conditions, using a suitable Lewis-acid catalyst such as aluminum chloride, compound (2) is internally cyclized to form compound (3). The reaction is preferably conducted at temperatures from about 0° C. to room temperature and allowed to proceed for about 24 hours.

Aminolysis of (3) to amide (4) can be achieved by treatment with concentrated ammonium hydroxide.

Alkylation of the hydroxyl of compound (4) can be readily achieved by treatment with an appropriate alkylating agent, such as Br(CH2)ₙY, where Y is —CO₂R, —PO₃R₂ or SO₃R and R is —(C₁-C₄)alkyl, to form intermediate (5). The reaction is preferably conducted in an aprotic polar solvent, such as dimethyl formamide, in the presence of potassium carbonate and a suitable catalyst, such as potassium iodide.

Conversion of (5) to the carboxylic or sulfonic acid or acid salt (6) may be achieved by treatment with an appropriate base, such as aqueous sodium hydroxide, in a polar protic solvent, such as methanol.

When n is 2, a bromoacetal must be employed as an alkylating agent to achieve the carboxylic acid (6). The alkylated moiety (5) is then converted to the acid (6) by oxidizing with sodium dichromatate in aqueous conditions.

When Y is —PO₃—, conversion to the acid (6), is preferably conducted in an alkyl halide solvent, such as methylene chloride, using a dealkylating agent, such as trimethylsilyl bromide, and an excess of potassium carbonate, followed by treatment with methanol.

When R¹, R², R³ or R⁴ are other than hydrogen, the preparation proceeds as described in Scheme IIi on the following page.

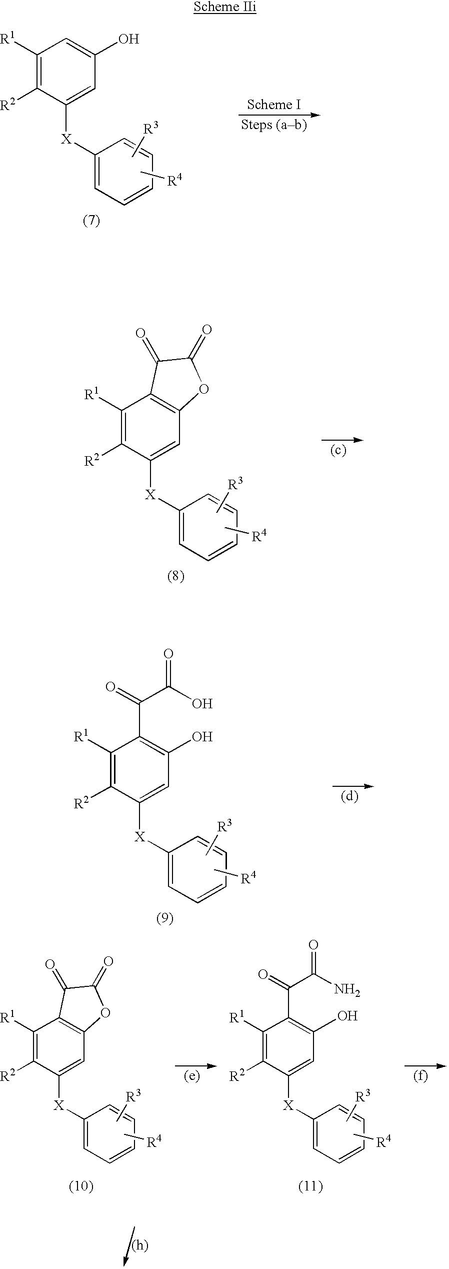

R′ is as defined in Scheme Ii.

An appropriately R¹, R² substituted phenol (7) is converted to lactone (8) following the procedures described in Scheme Ii, steps (a-b) above.

Conversion to the intermediate (9) is accomplished by reacting (2a) with an aqueous acid, such as hydrochloric acid which affords removal of aluminum chloride from the reaction. Acid (9) is converted to the corresponding acid chloride using oxalyl chloride with dimethyl formamide as a catalyst. The acid chloride is recyclized to the lactone (10) on removal of the solvent, preferably under vacuum. The lactone (10) is converted to the glyoxamide (11) by treatment with an excess of ammonia as described in Schemet †I, step (c), above.

Alkylation of (11) to prepare the ester (12), followed by conversion to the acid is accomplished according to the procedure outlined in Scheme I, steps (d) and (e).

Alternately, conversion of (10) to (12) can be accomplished in a one-pot procedure by treating the lactone (10) with sodium amide in an aprotic polar solvent, such as dimethylformamide, preferably at temperatures of from about 0° C. to 20° C., followed by alkylation with an appropriate alkyl halide.

j) Pyrrole sPLA₂ inhibitors and methods of making them are disclosed in U.S. patent applicaton Ser. No. 08/985,518 filed Dec. 5, 1997 (titled, “Pyrroles as sPLA₂ Inhibitors”), the entire disclosure of which is incorporated herein by reference.

The compositions and method of the invention for treatment of a mammal, including a human, afflicted with sepsis may be practiced with a pyrrole sPLA₂ described as follows:

A compound of the formula (Ij)

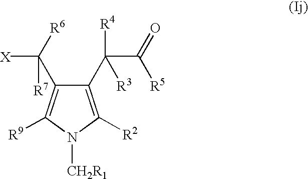

R¹ is hydrogen, (C₁-C₄)alkyl, phenyl or phenyl substituted with one or two substituents selected from the group consisting of —(C₁-C₄)alkyl, (C₁-C₄)alkoxy, phenyl (C₁-C₄)alkyl, (C₁-C₄)alkylthio, halo and phenyl;

R² is hydrogen, —(C₁-C₄)alkyl, halo, (C₁-C₄)alkoxy or (C₁-C₄)alkylthio;

R³ and R⁴ are each hydrogen or when taken together are ═O;

R⁵ is —NH₂ or —NHNH_{2;}

R⁶ and R⁷ are each hydrogen or when one of R⁶ and R⁷ is hydrogen, the other is —(C₁-C₄)alkyl, —(CH₂)ₙR¹⁰ where R¹⁰ is —CO₂R¹¹, —PO₃(R¹¹)₂, —PO₄(R¹¹)₂ or —SO₃R¹¹ where R¹¹ is independently hydrogen or —(C₁-C₄)alkyl and n is 0 to 4; or R⁶ and R⁷, taken together, are ═O or ═S;

X is R⁸(C₁-C₆)alkyl; R⁸(C₂-C₆)alkenyl or phenyl substituted at the ortho position with R⁸ where R⁸ is (CH₂)ₙR¹⁰ where R¹⁰ is —CO₂R¹¹, —PO₃(R¹¹)₂, —PO₄(R¹¹) or —SO₃R¹¹, R¹¹ and n is 1 to 4 as defined above, and additionally substituted with one or two substituents selected from the group consisting of hydrogen, —(C₁-C₄)alkyl, halo, (C₁-C₄)alkoxy, or two substituents which, when taken together with the phenyl ring to which they are attached, form a naphthyl group; and

R⁹ is hydrogen or methyl or ethyl;

or a pharmaceutically acceptable salt thereof.

Preferred pyrrole sPLA₂ inhibitors useful in the method of the invention are compounds of formula Ij wherein;

R¹ is phenyl;

R² is methyl or ethyl;

R⁵ is —NH_{2;}

R⁶ and R⁷ are each hydrogen;

X is R⁸(C₁-C₆)alkyl or phenyl substituted at the ortho position with R⁸ where

R⁸ is —CO₂R¹¹; and

R⁹ is methyl or ethyl.

A specific suitable pyrrole sPLA₂ inhibitors useful in the method of the invention is 2-[1-benzyl-2,5-dimethyl-4-(2-carboxyphenylmethyl)pyrrol-3-yl]glyoxamide.

The pyrrole compounds are prepared as follows:

Compounds of formula I where R⁵ is —NH₂ can be prepared as shown in Scheme Ij, below.

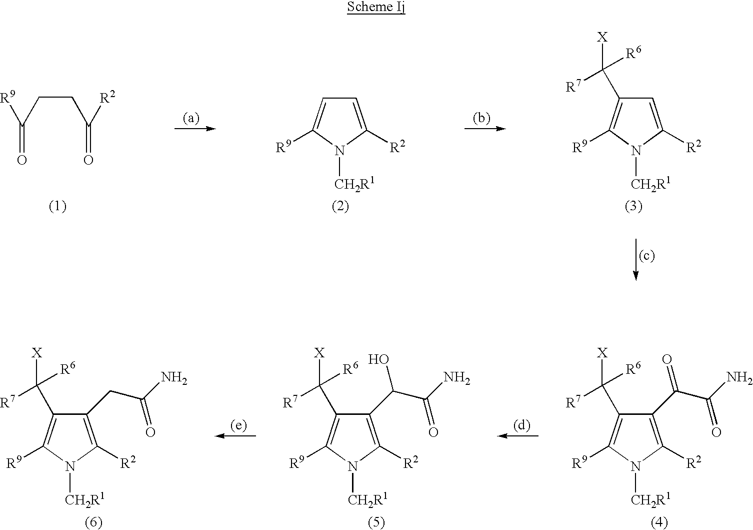

An appropriately substituted gamma-diketone (1) is reacted with an alkylamine of the formula NHCH₂R¹ to give pyrrole (2). Under Friedel-Crafts conditions, using a suitable Lewis-acid catalyst such as stannic chloride, aluminum chloride, or titanium tetrachloride (preferably stannic chloride) pyrrole (2) is ring alkylated with an alkyl or arylalkyl halide compound of the formula ZCR⁶R⁷X where Z is a suitable halogen and R⁸ of X is a protected acid or ester. The reaction is preferably conducted in a halogenated hydrocarbon solvent, such as dichloromethane, at ambient temperatures and allowed to proceed for from about 1 to about 24 hours.

Intermediate (3) is converted to (4) by sequential treatment with oxalyl chloride followed by ammonia. Selective reduction of (4) is accomplished in a two step process. In a hydride reduction using, for example, sodium borohydride, the hydroxy intermediate (5) is prepared which can be further reduced using either catalytic or hydride reduction (preferably palladium on carbon) to prepare (6). Deprotection of R⁸ to the acid may be readily achieved by conventional techniques. For example, when an alkyl ester is used as a protecting group, deprotection can be accomplished by treatment with a base, such as sodium hydroxide.

k) Naphthyl glyoxamide sPLA₂ inhibitors and methods of making them are described in U.S. patent application Ser. No. 09/091,079, filed Dec. 9, 1966 (titled, “Naphthyl Glyoxamides as sPLA2 Inhibitors”), the entire disclosure of which is incorporated herein by reference.

The compositions and method of the invention for treatment of a mammal, including a human, afflicted with sepsis may be practiced with a naphthyl glyoxamide sPLA₂ inhibitors described as follows:

A naphthyl glyoxamide compound or a pharmaceutically acceptable salt, solvate or prodrug derivative thereof; wherein said compound is represented by the formula Ik

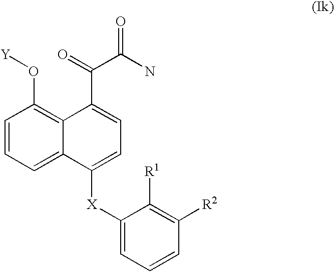

wherein:

R¹ and R² are each independently hydrogen or a non-interfering substituent with the proviso that at least one of R¹ or R² must be hydrogen;

X is —CH₂— or —O—; and

Y is (CH₂)ₙZ where n is a number from 1-3 and Z is an acid group selected from the group consisting of CO₂H, —SO₃H or —PO(OH)₂.

A specific suitable naphthyl glyoxamide sPLA₂ inhibitors useful in the method of the invention has the following structural formula:

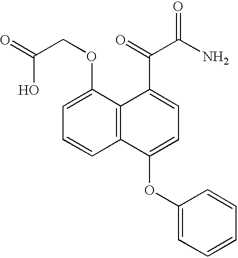

The naphthyl glyoxamide compounds are prepared as follows:

Compounds of formula I where X is oxygen can be prepared by the following reaction Scheme Ik.

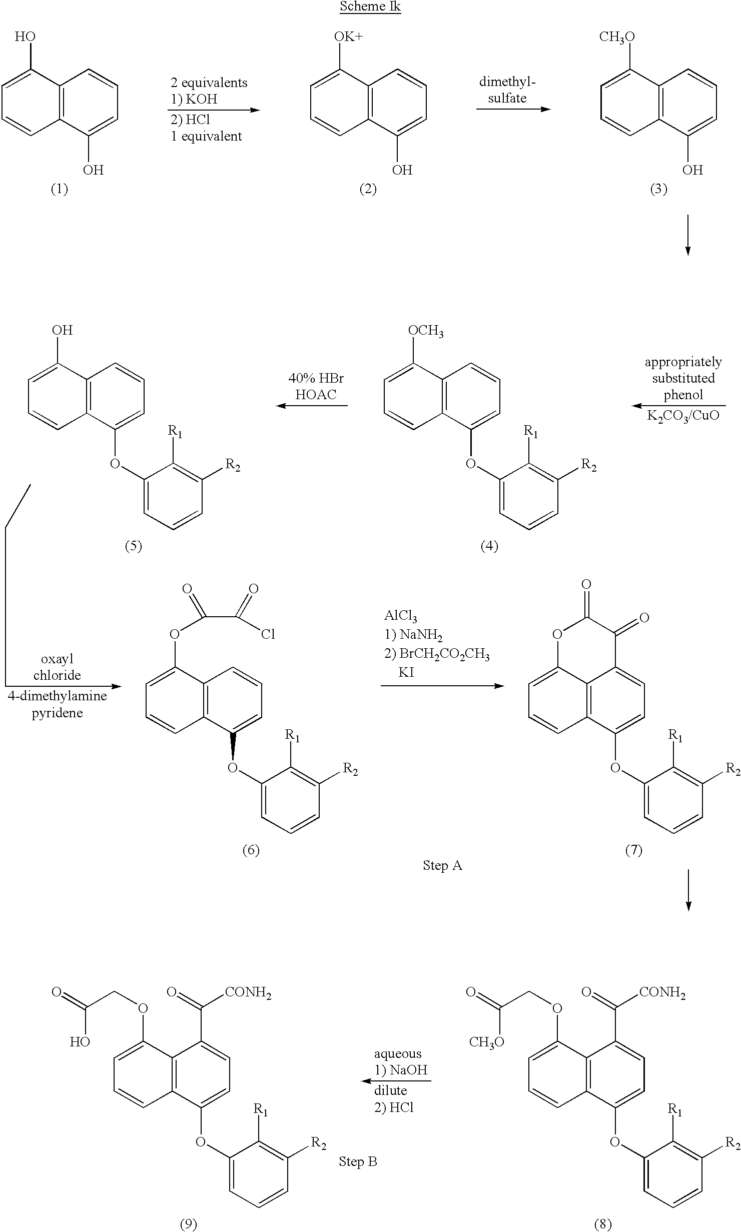

In the above depicted reaction scheme, the 1,5-dihydroxy napthalene starting material (1) is dispersed in water and then treated with 2 equivalents of potassium hydroxide. The resultant solution is chilled in an ice bath and one equivalent of a strong mineral acid, such as hydrochloric acid, is added to produce the potassium salt†(2).

Alkylation of the radical (2) can then be accomplished by treatment with a methylating agent such as dimethyl sulfate to prepare the ether (3).

Preparation of (4) is achieved by reacting the ether (3) with an appropriately substituted phenol in an Ullman-type reaction using potassium carbonate and cupric oxide.

De-methylation of (4) can be accomplished by treating (4) with a 40% HBr/HOAC solution at reflux in a protic polar solvent such as acetic acid, to prepare (5).

Reflux of compound (5) with oxalyl chloride and 4-demethylamino pyridine, in an alkylhalide solvent such as methylene chloride, prepares the oxalyl chloride (6).

Internal cyclization of (6) can be achieved under Friedel-Crafts condition using aluminum chloride or other similar metal halide as the catalyst. The reaction can be conveniently conducted in an alkyl halide solvent, such as 1, 2-dichloro ethane.

Alkylation and hydrolysis of the cyclized compound (7) can be achieved by reacting (7) with an alkaliamide base, such as sodium amide, followed by treatment with an alkylating agent, such as methyl bromoacetate, using potassium iodide as a catalyst.

Finally, the acid (9) is achieved by treating the ester (8) with an alkali base, such as aqueous sodium hydroxide, followed by treatment with a dilute aqueous mineral acid such as hydrochloric acid The acid compound (9) is then extracted with an organic solvent such as ethyl acetate.

The final product (9) can be purified using standard recrystallization procedures in a suitable organic solvent such as methylene chloride/hexane.

Compounds of formula I where X is methylene can be prepared as shown in the following Scheme IIk

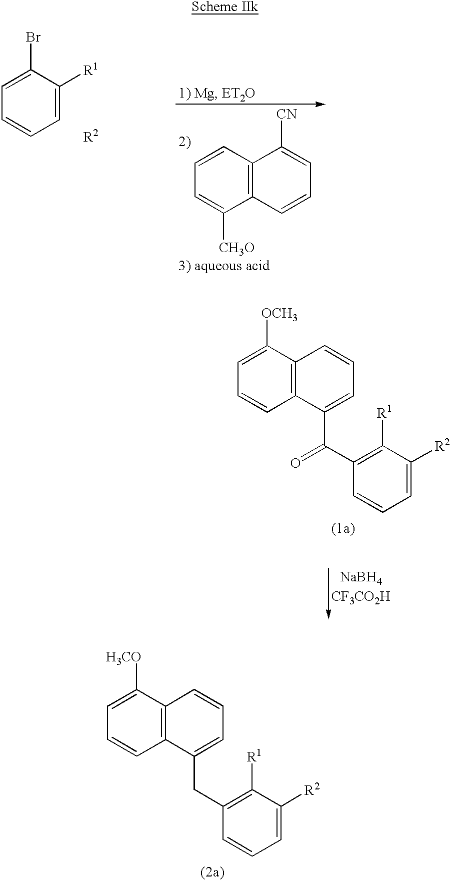

Using an appropriately substituted phenyl bromide, a Grignard reagent is prepared. The phenyl Grignard is then reacted with 4-methoxy naphthylnitrile and the resultant compound is hydrolyzed with a dilute acid such as hydrochloric acid to form the benzoyl naphthylene compound (1a).

Reduction of (1a) to form compound (2a) is accomplished by treatment with a reducing agent such as sodium borohydride. The reaction is conducted in a solvent-catalyst such as trifluoroacetic acid and initiated in an ice bath which is allowed to warm to room temperature as the reaction proceeds.

The desired naphthyl glyoxamide may then be prepared from (2a) according to the procedure in Scheme I starting with the chloromethylation step.

It will be readily appreciated by a person skilled in the art that the substituted benzyl bromide, substituted phenol and substituted naphthylnitrile compounds of Schemes I and II are either commercially available or can be readily prepared by known techniques from commercially available starting materials.

l) Phenyl acetamide sPLA₂ inhibitors and methods of making them are disclosed in U.S. patent application Ser. No. 08/976,858, filed Nov. 24, 1997 (titled, “Phenyl Acetamides as sPLA₂ Inhibitors”), the entire disclosure of which is incorporated herein by reference.

The compositions and method of the invention for treatment of a mammal, including a human, afflicted with sepsis may be practiced using a phenyl acetamide SPLA₂ inhibitor represented by formula (Il) as follows:

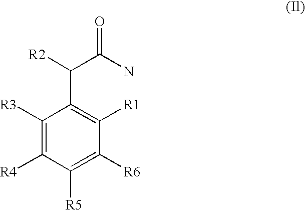

wherein:

R¹ is —H or —O(CH₂)ₙZ;

R² is —H or —OH;

R³ and R⁴ are each independently —H, halo or —(C₁-C₄)alkyl;

One of R⁵ and R⁶ is —YR⁷ and the other is —H, where Y is —O— or —CH₂— and R⁷ is phenyl or phenyl substituted with one or two substituents selected from the group consisting of halo, —(C₁-C₄)alkyl, (C₁-C₄)alkoxy, phenyl or phenyl substituted with one or two halo groups;

Z is —CO₂R, —PO₃R₂ or —SO₃R where R is —H or —(C₁-C₄)alkyl; and

n is 1-8;

or a pharmaceutically acceptable salt, racemate or optical isomer thereof;

provided that when R⁶ is YR⁷, R¹ is hydrogen; and

when R¹, R², R³, R⁴ and R⁶ are hydrogen and R⁵ is YR⁷ where Y is —O—, R⁷ cannot be phenyl; and

when R¹, R², R³, R⁴ and R⁶ are hydrogen, R⁵ is YR⁷ where Y is CH₂, R⁷ cannot be phenyl substituted with one methoxy or two chloro groups.

Preferred suitable phenyl acetamide SPLA₂ inhibitors useful in the composition and method of the invention are as follows:

Compounds of formula I wherein R², R³ and R⁴ is H, Y is oxygen or CH₂, R⁷ is phenyl or phenyl substituted at the meta position with one or two substituents selected from halo, —(C₁-C₄)alkyl, (C₁-C₄)alkoxy, phenyl or phenyl substituted with halo and n is 4-5.

A specific suitable phenyl acetamide sPLA₂ inhibitor useful in the method of the invention is 2-(4-carboxybutoxy)-4-(3-phenylphenoxy) phenylacetamide.

The phenyl acetamide inhibitors are prepared as follows:

Compounds of formula I where R¹ and R² are H, R⁵ or R⁶ are YR⁷ where R⁷ is phenyl or substituted phenyl and Y is oxygen can be prepared as illustrated in Scheme Il (a), below.

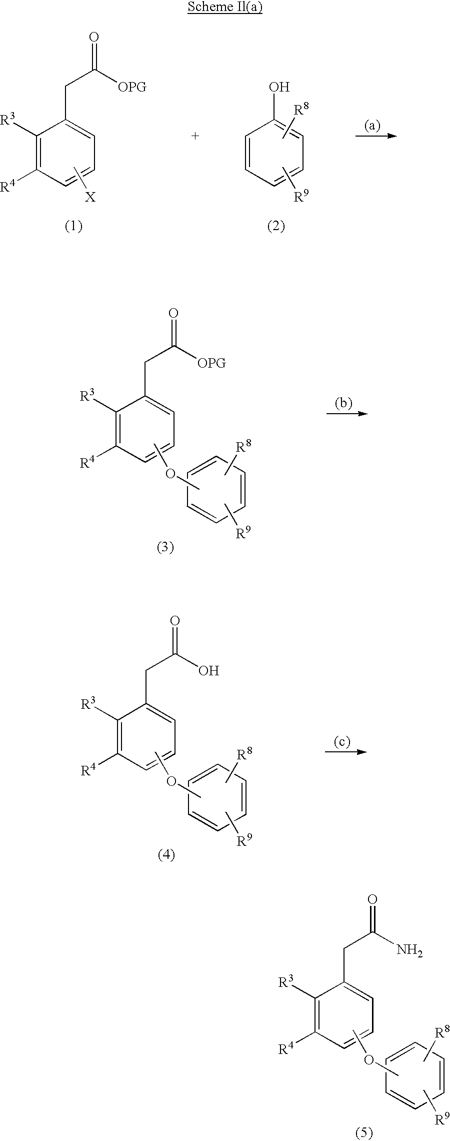

X is halo;

R⁸ and R⁹ are each independently —H, halo, —(C₁-C₄)alkyl, (C₁-C₄)alkoxy, phenyl or phenyl substituted with one or two halo groups; and

PG is a carboxyl protecting group

An appropriately substituted carboxy-protected halophenyl compound (1), where the halogen is preferably bromine, is coupled with an appropriately substituted phenol (2) under modified Ullmann conditions, by refluxing with potassium carbonate and cupric oxide in an aprotic polar solvent, such as pyridine, under an inert gas such as argon. The reaction is substantially complete in 1-24 hours.

Intermediate (3) is deprotected by treatment with a base such as aqueous potassium hydroxide using a solvent, such as diethylene glycol. The reaction, preferably conducted at about 100°-150° C., is substantially complete in 1-24 hours.

Conversion to the amide (5) can then be readily achieved by treatment first with oxalyl chloride in an alkyl halide solvent, such as methylene chloride, using dimethylformamide as a catalyst, at temperatures of from about 0° C. to ambient temperature, followed by treatment with an excess of ammonia gas, again in an alkyl halide solvent.

Alternately, compounds of formula I can be prepared according to the procedure of Scheme I(b), below.

The substituted phenol (2) is coupled with an appropriately substituted benzyl halide (6) as described in Scheme I(a), step a, above, to prepare (7).

Halogenation of (7) is achieved using a halogenating agent, such as N-bromosuccinimide and a catalyst, such as 2,2′azobisisobutyronitrile, in an alkyl halide solvent, such as chloroform, to prepare (8).

Treatment of (8) with sodium cyanide in an aprotic polar solvent, such as dimethyl formamide produces the nitrile (9) which can then be readily converted to the amide (10) by treatment with an aqueous acid, such as hydrochloric acid.

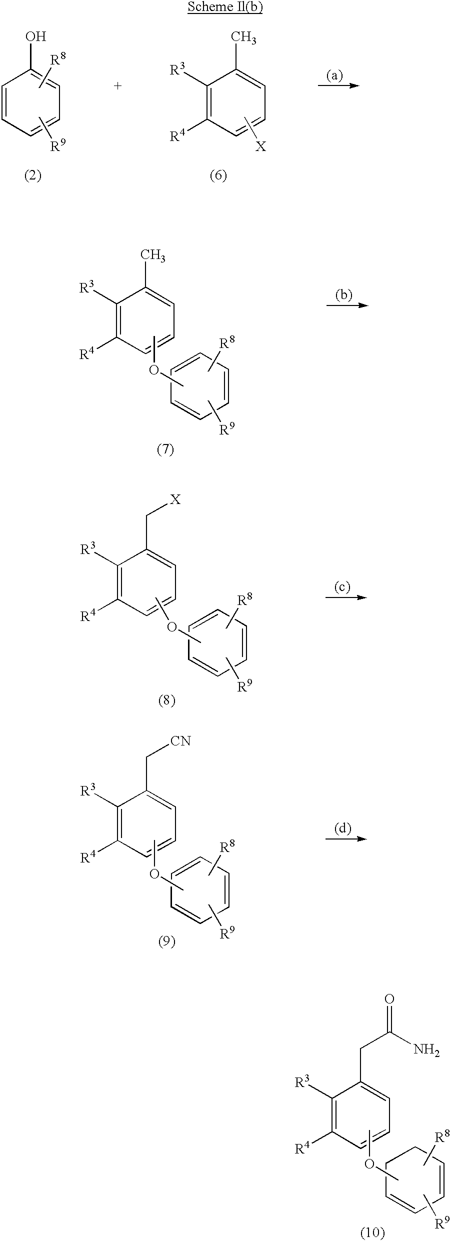

R⁸ and R⁹ are as shown in Scheme I(a),

X is halo.

In another procedure, compounds of formula I where R¹, R², R³ and R⁴ are hydrogen, Y is —O— or —CH₂— and R⁷ is phenyl can be prepared as portrayed in Scheme IIl.

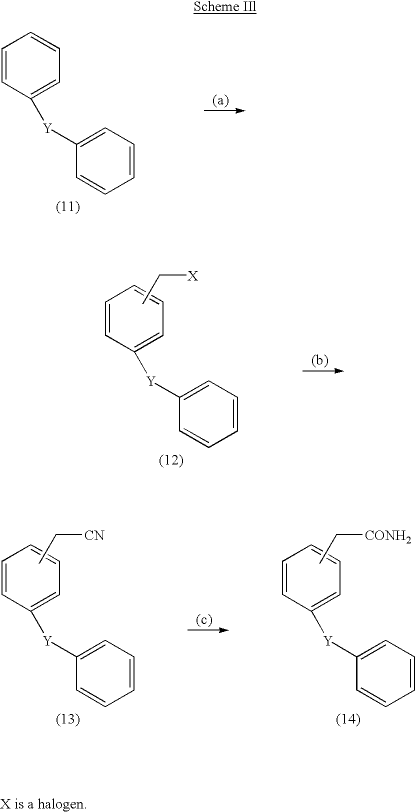

An appropriate diphenyl compound (11) is treated with paraformaldehyde and a halogenating agent, such as 40% hydrogen bromide in acetic acid. Two positional isomers result with the X substituent at either the meta or para position of the phenyl ring to which it is attached.

Displacement of the halogen to prepare the nitrile isomers (13) can be achieved by treatment of (12) with sodium cyanide in dimethylformamide as described in Scheme †I(b), step (c), above. The isomers can then be readily separated by conventional chromatographic techniques and each isomer may be converted to its respective amide (14) by treatment with hydrogen peroxide and potassium carbonate in an aprotic polar solvent, such as dimethylsulfoxide.

Compounds where R¹ is —O(CH₂)ₙZ can be prepared as illustrated in Scheme IIIl, below.

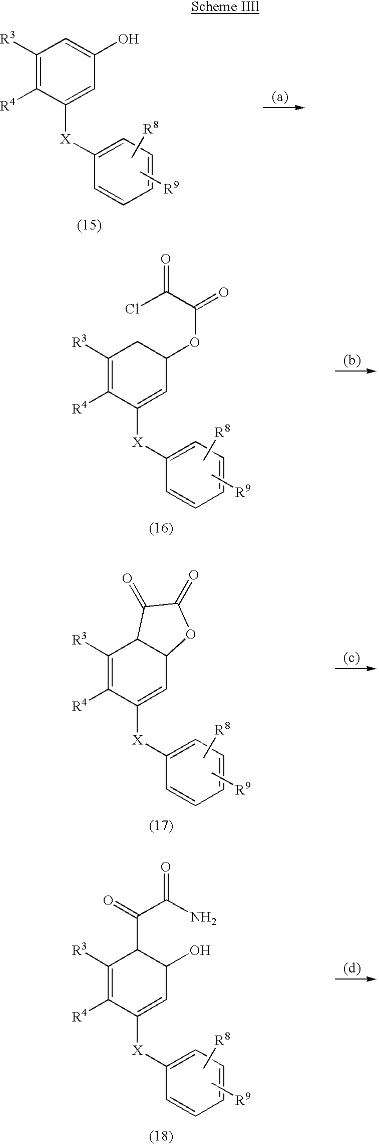

Intermediate (16) is prepared by refluxing an appropriately substituted diphenyl compound (15) with oxalyl chloride in an alkyl halide solvent, such as chloroform. Preferably the reaction is catalyzed with 4,4-N-dimethylaminopyridine.

Cyclization to the lactone (17) can be achieved under Friedel-Crafts conditions using a suitable metal halide, such as aluminum chloride, as the catalyst. Conversion to the glyoxamide (18) can be achieved by aminolysis of the lactone ring using concentrated ammonium hydroxide.

Alkylation of the hydroxy group to prepare the desired alkyl-linked ester (19) occurs by treatment of (18) with an appropriate alkylating agent, such as (X)(CH₂)ₙB where B is CO₂PG, —PO₃PG or —SO₃PG, X is halo and PG is an acid protecting group, preferably methyl.

Partial reduction of the carbonyl in the glyoxamide (19) is achieved by treatment with a suitable reducing agent, such as sodium borohydride in methanol, preferably at temperatures of from 0°-20° C., to prepare the intermediate (20). The desired acid or acid salt (21) can be accomplished by treatment with a suitable base, such as sodium hydroxide.

Further reduction of intermediate (20) can be achieved by treatment with triethylsilane in a strong acid, such as trifluroacetic acid, under an inert gas, such as argon, to prepare (22) followed, again, by conversion to the acid or salt (23) with a strong base.

m) Naphthyl acetamide sPLA₂ inhibitors and the method of making them are described in U.S. patent application Ser. No. 09/091,077, filed Dec. 9, 1996 (titled, “Benzyl naphthalene sPLA₂ Inhibitors”), the entire disclosure of which is incorporated herein by reference.

The composition and method of the invention for treatment of a mammal, including a human, afflicted with sepsis is practiced using a naphthyl acetamide sPLA₂ inhibitor represented by formula (Im)as follows:

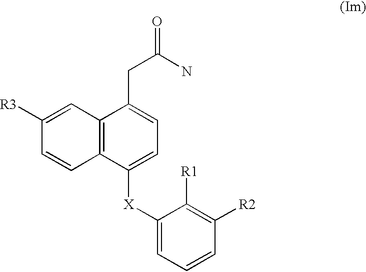

wherein:

R¹ and R² are each independently hydrogen or a non-interfering substituent with the proviso that at least one of R¹ and R² must be hydrogen;

R³ is hydrogen, —O(CH₂)ₙY,

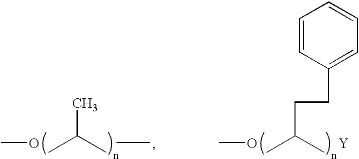

where n is from 2 to 4 and Y is —CO₂H, —PO₃H₂ or SO₃H; and

X is —O— or —CH₂—.

Compounds where X is oxygen can be prepared by the following Scheme Im.

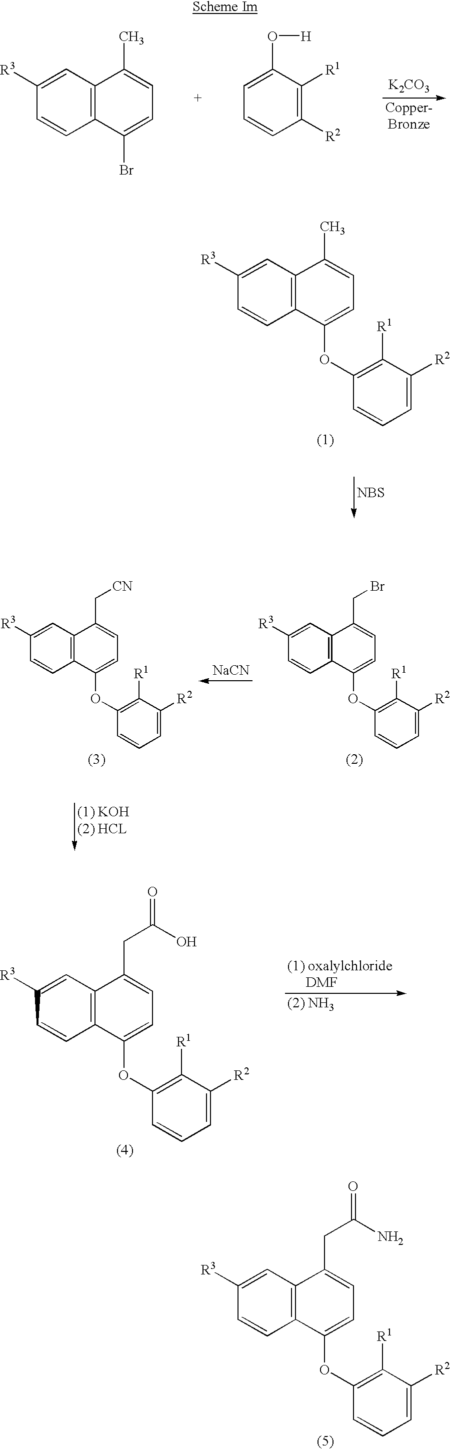

In the first step of the above reaction scheme, an appropriately substituted 1-bromo-4-methylnapthalene and an appropriately substituted phenol are dissolved in an aprotic polar solvent such as pyridine. The mixture is treated with an excess of potassium carbonate and an excess of copper-bronze and refluxed under a nitrogen blanket to produce (1).

Bromination of compound (1) to produce (2) is accomplished by refluxing (1) with a brominating agent, such as N-bromosuccinamide, in a non-polar alkyl halide solvent, such as carbon tetrachloride, using 2,2-azobisisobutyronitrile as a catalyst.

Treatment of (2) with sodium cyanide produces (3). This reaction is best conducted in an aprotic polar solvent, such as dimethyl sulfoxide (DMSO), while heating to a temperature of about 60° C.

Hydrolysis of the cyano compound (3) to produce the acid (4) is accomplished in two steps. Using a polar protic solvent, such as diethylene glycol as a cosolvent, the cyano compound (3) is treated with an alkali metal base, such as potassium hydroxide, and the mixture is heated to about 90-95° C. The resultant product is then reacted with a strong mineral acid such as hydrochloric acid.

Conversion of (4) to the desired naphthyl acetamide compound (5) is accomplished by another two-step process. First, the acid (4) is dissolved in an alkyl halide solvent such as methylene chloride. The acid/alkyl halide solution is chilled in an ice bath then treated with oxalyl chloride, using dimethylformamide (DMF) as a catalyst, to produce the acid chloride. The solution is allowed to warm to room temperature and then treated with ammonia gas at room temperature to produce (5).

The desired product (5) can be purified using standard recrystallization procedures in a suitable organic solvent, preferably methylene chloride/hexane.

Compounds where X is methylene can be prepared by the following Scheme IIm

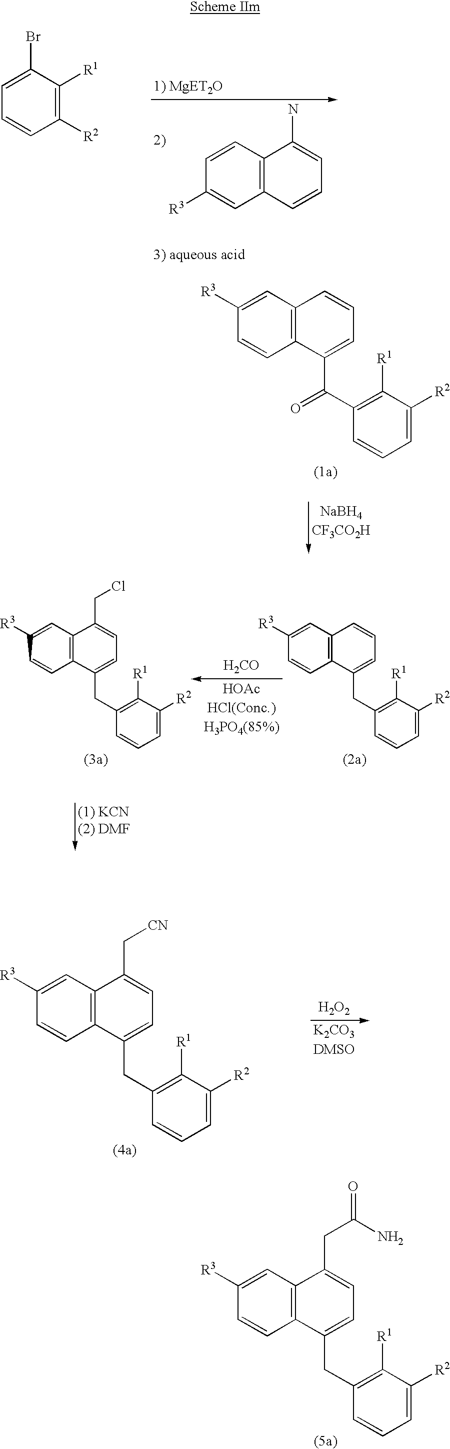

Compound (1a) is prepared by a grignard reaction. The Grignard reagent starting material is prepared by reacting an appropriately substituted phenyl bromide with magnesium and ether. The reagent is then reacted with an appropriately substituted naphthyl nitrile and the resultant compound is hydrolyzed with an aqueous acid such as hydrochloric acid to form the benzoyl napthyl (1a).

Reduction of (1a) is accomplished by treatment with a molar excess of a reducing agent such as sodium borohydride. The reaction is initiated in an ice bath using a solvent-catalyst such as trifluoroacetic acid and then allowed to warm to room temperature as the reduction proceeds.

Chloromethylation of (2a) is achieved by treatment with an excess of formaldehyde and concentrated hydrochloric acid in a polar acidic solvent such as an acetic/phosphoric acid mixture. The reaction is best conducted at a temperature of about 90° C.

The nitrile 4(a) is prepared by a nucleophilic displacement of the chloride compound (3a)with cyanide. The reaction is conducted by refluxing (3a) with a slight molar excess in an aprotic polar solvent of sodium cyanide such as dimethylformamide (DMF) for about five hours, then allowing the reaction to continues while it cools to room temperature.

The desired naphthylamide (5a) is then prepared from the nitrile (4a) in a three-step process. To a solution of nitrile (4a), dissolved in an aprotic polar solvent such as DMSO, potassium carbonate is added to make the nitrile solution slightly basic. Hydrolysis of the nitrile is then achieved by treatment with an aqueous hydrogen peroxide solution. Crystallization of the naphthyl acetamide may be accomplished by adding water to the peroxide solution.

Compounds where R³ is other than hydrogen can be readily prepared by using a 1-bromo-4-methyl-napthalene with a protected phenol, such as a methoxy group, on the 6-position of the napthalene ring as a starting material. The process is conducted, as described above, to prepare compounds (1)-(3). Acid hydrolysis of the cyano group (3) and deprotection of the protected phenol can be accomplished by treating (3) with a 40% hydrogen bromide solution in acetic acid. The deprotected phenol can then be reacted to prepare the appropriate substituent at the 6-position of the napthyl ring. For example, preparation of compounds where R³ is —O(CH₂)ₙCOOH can be achieved by alkyalting the phenol with an appropriate alkyl halide followed by conversion to the acid by treatment with a base such as aqueous sodium hydroxide followed by dilute hydrochloric acid.

It will be readily appreciated by one skilled in the art that the substituted phenol and phenyl bromide starting materials are either commercially available or can be readily prepared by known techniques from commercially available starting materials. All other reactants and reagents used to prepare the compounds of the present invention are commercially available.

Most Preferred sPLA₂ inhibitors:

1H-indole-3-glyoxylamide sPLA₂ inhibitors and carbazole sPLA2 inhibitors (as described, supra.) are most preferred for the compositions and method this invention.

IV. Pharmaceutical Compositions of the Invention

The pharmaceutical composition of the invention comprises as essential ingredients:

(i) neutrophil elastase inhibitor, and

(ii) an sPLA₂ inhibitor.

When the pharmaceutical composition of the invention is prepared in injectable form it is a composition comprising as ingredients:

(a) a neutrophil elastase inhibitor,

(b) an sPLA₂ inhibitor, and

(c) an injectable liquid carrier.

a. Ratio and Amount of Ingredients in the Composition of the Invention

The essential ingredients (a) a neutrophil elastase inhibitor and (b) an sPLA₂ inhibitor are present in the formulation in such proportion that a dose of the formulation provides a pharmaceutically effective amount of each ingredient to the patient being treated.

Co-Agents for the Composition of the Invention:

The essential neutrophil elastase inhibitor and sPLA2 inhibitor ingredients of the invention may additionally be supplemented by the including a therapeutically effective amount of Activated Protein C, a serine protease particularly useful for treating sepsis (inclusive of severe sepsis). The identity and preparation of Activated Protein C is described in U.S. Pat. Nos. 4,775,624; 4,981,952; 4,992,373; the disclosures of which are incorporated herein by reference.

The resultant ternary composition contains as active ingredients:

1) neutrophil elastase inhibitor;

2) sPLA2 inhibitor,

3) Activated Protein C., and

4) optional carriers and/or diluents.

The dose of composition of the invention to be administered is determined depending upon age, body weight, symptom, the desired therapeutic effect, the route of administration, and the duration of the treatment etc. Typically, the weight ratio of neutrophil elastase inhibitor to an sPLA2 inhibitor from 100:1 to 1:100 and preferably from 10:1 to 1:10.

An effective dosage of an SPLA₂ inhibitor in human patients is considered to be between 0.01 and 5000 (milligrams/kg/day). Preferably, the dosage is between 0.1 to 100 (milligrams/kg/day).

For the neutrophil elastase inhibitor, in the human adult, the doses per person for one time are generally for intravenous administration between 1 to 5000 mg./day, and preferably from 250 to 500 mg./day. The dose per person for oral one time administration is from 1 to 50000 mg./day and preferably from 500 to 5000 mg./day. Dosing may be once or several times a day.

In making compositions of the invention the essential ingredients; neutrophil elastase inhibitor and sPLA₂ inhibitor are co-present and may be mixed in any homogeneous or non-homogeneous manner or adjacently or otherwise promixately placed together in an individual dosage unit suitable for practicing the method of the invention.

The dosage unit of the neutrophil elastase inhibitor will usually be admixed with a carrier or inert ingredients, or diluted by a carrier, or enclosed within a carrier which may be in the form of a ampoule, capsule, time release dosing device, sachet, paper or other container. When the carrier serves as a diluent, it may be a solid, semi-solid, paste, or liquid material which acts as a vehicle, or can be in the form of tablets, pills, powders, lozenges, elixirs, suspensions, emulsions, solutions, syrups, aerosols (as a solid or in a liquid medium), or ointment, containing, for example, up to 10% by weight of the active compound.

The dosage unit of the an sPLA₂ inhibitor will usually be admixed with a liquid carrier and/or other inert ingredients or enclosed within a carrier which may be in the form of a ampoule, bottle, time release dosing device or other container. When the carrier serves as a diluent, it may be a liquid material which acts as a vehicle, or can be in the form of solutions containing, for example, up to 10% by weight of the active compound.

For the pharmaceutical formulations containing both (a) neutrophil elastase inhibitor and (b) an sPLA₂ inhibitor the carrier may be an injectable liquid medium such as is well known in the art. The injectable liquid must be such that permits parenteral administration, that is, introduction of substances to a mammal being treated by intervenous, subcuataneous, intramuscular, or intramedullary injection. Intravenous injection is most preferred as a means of administration.

The Active ingredient can be dissolved or suspended in a pharmaceutically acceptable carrier, such as sterile water, sterile water containing saline and/or sugars and/or suspension agents or a mixture of both. For example, for intravenous injection the compounds of the invention may be dissolved in at a concentration of 2 mg/ml in a 4% dextrose/0.5% Na citrate aqueous solution.

Liquid compositions for oral administration include pharmaceutically-acceptable emulsions, solutions, suspensions, syrups and elixirs containing inert diluents commonly used in the art such as distilled water or ethanol. Besides inert diluents such compositions may also comprise adjuvants such as wetting and suspending agents, and sweetening, flavouring, perfuming and preserving agents.

Other compositions for oral administration include spray compositions which may be prepared by known methods and which comprise one or more of the active compound(s). Besides inert diluents such compositions may also comprise stabilizers such as sodium bisulfite and buffer for isotonicity, for example sodium chloride, sodium citrate or citric acid.

The manufacturing methods of spray compositions for inhalation therapy have been described in detail, for example, in the specifications of U.S. Pat. No. 2,868,691 and U.S. Pat. No. 3,095,355.

Preparations for injection according to the present invention for parenteral administration include sterile aqueous or non-aqueous solutions, suspensions or emulsions. Example of aqueous solvents or suspending media are distilled water for injection and physiological salt solution. Examples of non-aqueous solvents or suspending media are propylene glycol, polyethylene glycol, vegetable oils such as olive oil, alcohols such as ehtanol, Polysorbate 80 (registered Trade Mark). These compositions may also include adjuvants such as preserving, wetting, emulsifying and dispersing agents stabilizing agents (e.g. lactose) and solubilizers (e.g. glutamic acid and asparaginic acid). They may be sterilized, for example, by filtration through a bacteria-retaining filter, by incorporation of sterilizing agents in the compositions or by irradiation. They may also be manufactured in the form of sterile solid compositions which can be dissolved in sterile water or some other sterile injectable medium immediately before use.

A solid carrier can be one or more substances which may also act as flavoring agents, lubricants, solubilizers, suspending agents, binders, tablet disintegrating agents and encapsulating material. Suitable solid carriers are magnesium carbonate, magnesium stearate, talc, sugar lactose, pectin, dextrin, starch, gelatin, tragacanth, methyl cellulose, sodium carboxymethyl cellulose, low melting waxes, and cocoa butter.

The sPLA₂ inhibitor and the neutrophil elastase inhibitor, either separately or together, may be in the form of powder, tablet or capsule. A solid carrier can be one or more substances which may also act as flavoring agents, lubricants, solubilizers, suspending agents, binders, tablet disintegrating agents and encapsulating material. Suitable solid carriers are magnesium carbonate, magnesium stearate, talc, sugar lactose, pectin, dextrin, starch, gelatin, tragacanth, methyl cellulose, sodium carboxymethyl cellulose, low melting waxes, and cocoa butter.

The following pharmaceutical formulations are useful (as stated) for either the sPLA₂ inhibitor alone, or the neutrophil elastase inhibitor alone, or as Active Ingredient which is a combination of (a) sPLA₂ inhibitor and (b) neutrophil elastase inhibitor.

Typically, from 10 mg to 1000 mg of the Active Ingredient inhibitor is used in a unit dose of the formulation. A patient may typically receive from 1 to 8 doses per day.

Quantity
(mg/capsule)

Formulation 1
Hard gelatin capsules are prepared using the
following ingredients:

| | | |
|---|---|---|
| Active Ingredient | 250 | |
| Starch, dried | 200 | |
| Magnesium stearate | 10 | |
| Total | 460 | mg |

Formulation 2
A tablet is prepared using the ingredients below:

| | | |
|---|---|---|
| Active Ingredient | 250 | |
| Cellulose, microcrystalline | 400 | |
| Silicon dioxide, fumed | 10 | |
| Stearic acid | 5 | |
| Total | 665 | mg |

The components are blended and compressed to form tablets each weighing 665 mg

Formulation 3
An aerosol solution is prepared containing the
following components:

| | Weight |
|---|---|
| Active Ingredient | 0.25 |
| Ethanol | 25.75 |
| Propellant 22 (Chlorodifluoromethane) | 74.00 |
| Total | 100.00 |

The Active Ingredient is mixed with ethanol and the mixture added to a portion of the propellant 22, cooled to −30° C. and transferred to a filling device. The required amount is then fed to a stainless steel container and diluted with the remainder of the propellant. The valve units are then fitted to the container.

Formulation 4
Tablets, each containing 60 mg of sPLA₂ inhibitor,
are made as follows:

| | |
|---|---|
| Active Ingredient | 60 mg |
| Starch | 45 mg |
| Microcrystalline cellulose | 35 mg |
| Polyvinylpyrrolidone (as 10% solution in water) | 4 mg |
| Sodium carboxymethyl starch | 4.5 mg |
| Magnesium stearate | 0.5 mg |
| Talc | 1 mg |
| Total | 150 mg |

The Active Ingredient starch and cellulose are passed through a No. 45 mesh U.S. sieve and mixed thoroughly. The aqueous solution containing polyvinylpyrrolidone is mixed with the resultant powder, and the mixture then is passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed on a tablet machine to yield tablets each weighing 150 mg.

Formulation 5
Capsules, each containing 80 mg of Active
Ingredient, are made as follows:

| | |
|---|---|
| Active Ingredient | 80 mg |
| Starch | 59 mg |
| Microcrystalline cellulose | 59 mg |
| Magnesium stearate | 2 mg |
| Total | 200 mg |

The Active Ingredient cellulose, starch, and magnesium stearate are blended, passed through a No. 45 mesh U.S. sieve, and filled into hard gelatin capsules in 200 mg quantities.

Formulation 6
Suppositories, each containing 225 mg of sPLA₂
inhibitor, are made as follows:

| | |
|---|---|
| Active Ingredient | 225 mg |
| Saturated fatty acid glycerides | 2,000 mg |
| Total | 2,225 mg |

The Active Ingredient is passed through a No. 60 mesh U.S. sieve and suspended in the saturated fatty acid glycerides previously melted using the minimum heat necessary. The mixture is then poured into a suppository mold of nominal 2 g capacity and allowed to cool.

Formulation 7
Suspensions, each containing 50 mg of Active
Ingredient per 5 ml dose, are made as follows:

| | |
|---|---|
| Active Ingredient | 50 mg |
| Sodium carboxymethyl cellulose | 50 mg |
| Syrup | 1.25 ml |
| Benzoic acid solution | 0.10 ml |
| Flavor | q.v. |
| Color | q.v. |
| Purified water to total | 5 ml |

The Active Ingredient is mixed with the sodium carboxymethyl cellulose and syrup to form a smooth paste. The benzoic acid solution, flavor and color are diluted with a portion of the water and added, with stirring. Sufficient water is then added to produce the required volume.

Formulation 8
An intravenous formulation may be prepared as
follows:

| | |
|---|---|
| Active Ingredient | 100 mg |
| Isotonic saline | 1,000 ml |

The solution of the above Active Ingredient generally is administered intravenously to a subject at a rate of 1 ml per minute.

Typically, from 10 mg to 1000 mg of the neutrophil elastase inhibitor is used in a unit dose of the formulation. The solution of the above Active Ingredient generally is administered intravenously to a subject at a rate of 1 ml per minute.

Typically, from 10 mg to 1000 mg of the Active Ingredient is used in a unit dose of the formulation.

A unit dosage formulation suitable for administration by continuous infusion is prepared by mixing at pH 6.0, an sPLA₂ inhibitor, a neutrophil elastase inhibitor, a salt (NaCl), a bulking agent (sucrose), and a buffer (citrate). The active ingredient, salt, and bulking agent are mixed in a weight to weight ratio of about 1 part Active ingredient, between about 7 and 8 parts salt, and between about 5 to 7 parts bulking agent. After mixing, the solution is transferred to vials and lyophilized. The vials comprising the active ingredients is sealed and stored until use.

V. Treating Respiratory Diseases and Inflammatory Diseases by The Method of the Invention

This invention is a method of treating or preventing Inflammatory Disease or Respiratory Disease by administering to a mammal in need thereof a therapeutically effective amount of (a) a neutrophil elastase inhibitor and a therapeutically effective amount of (b) an sPLA₂ inhibitor; wherein (a) and (b) are both administered within a therapeutically effective interval. The administration of (a) or (b) to a septic patient may be either continuous or intermittent.

A. Method of the Invention using simultaneous delivery of an sPLA₂ inhibitor and neutrophil elastase inhibitor The an sPLA₂ inhibitor and a neutrophil elastase inhibitor can be delivered simultaneously. One convenient method of simultaneous delivery is to use the compositions of the invention described in section IV, supra, wherein the Active ingredient has the essential ingredients co-present in a unit dosage form. Solution or suspensions of mixed essential ingredients may, if desired, be delivered from the same IV liquid holding bag. Another method of simultaneous delivery of the an sPLA₂ inhibitor and a neutrophil elastase inhibitor is to deliver them to the patient separately but simultaneously. Thus, for example, the neutrophil elastase inhibitor may be given as an oral formulation at the same time the an sPLA₂ inhibitor is given parenterally. Dosage of a neutrophil elastase inhibitor can begin simultaneously with the an sPLA₂ inhibitor administration. The length of the neutrophil elastase inhibitor administration can extend past the an sPLA₂ inhibitor administration.

B. Method of the Invention using non-simultaneous delivery of an sPLA₂ inhibitor and neutrophil elastase inhibitor.

Each of the essential ingredients, viz., a therapeutically effective amount of (a) a neutrophil elastase inhibitor and a therapeutically effective amount of (b) an sPLA₂ inhibitor have a therapeutically effective interval, namely the interval of time in which each agent provides benefit for the patient being treated with Inflammatory Disease or Respiratory Disease. The method of the invention may be practiced by separately dosing the patient in any order with a therapeutically effective amount of (a) a neutrophil elastase inhibitor and a therapeutically effective amount of (b) an sPLA₂ inhibitor provided that each agent is given within the period of time that that the other agent is therapeutically effective against Inflammatory Disease or Respiratory Disease or organ failure resulting from these pathologic processes.

Typically, intravenous forms of neutrophil elastase inhibitor, for example, sodium N-[2-[4-(2,2-dimethylpropionyloxy)phenylsulfonyl-amino]benzoyl]aminoacetate tetrahydrate, are therapeutically effective immediately upon administration and up to 5 days later, and preferably in the time interval from 5 minutes after administration to 72 hours after administration. Similarly, salts of N-[2-[[[4-(2,2-dimethyl-1-oxopropoxy)phenyl]sulfonyl]amino]benzoyl]-glycine (CAS Registration No. 127373-66-4) may be used as oral forms of neutrophil elastase inhibitor and typically therapeutically effective from about 10 minutes to 5 days, and preferably from one-half hour to 72 hours after administration.

Dosage delivery of the neutrophil elastase inhibitor can begin up to 48 hours prior to the an sPLA₂ inhibitor infusion with the preferred time being up to 24 hours and the most preferred being up to 12 hours. Alternatively, dosage of a neutrophil elastase inhibitor can begin up to 48 hours after the initiation of the an sPLA₂ inhibitor infusion with the preferred time being up to 24 hours after and the most preferred being up to 12 hours after. The neutrophil elastase inhibitor and/or an sPLA₂ inhibitor can be independently administered by a variety of routes including oral, aerosol, rectal, transdermal, subcutaneous, intravenous, intramuscular, and intranasal, injectable solution and by other routes including oral, aerosol and intranasal. The Active Ingredient, however, is preferably administered parenterally to a septic patient to insure delivery into the bloodstream in an effective form as fast as possible.

VI. Duration of Treatment for patients having Inflammatory Diseases or Respiratory Diseases using the Method of the Invention

The amount and relative ratio of an sPLA2 inhibitor and neutrophil elastase inhibitor to be used in the practice of the method of invention is set out in the previous section, (V) supra. It may be appreciated that it may be necessary to make routine variations to the dosage of either agent depending on the age and condition of the patient.

The decision to begin the therapy will be based upon the appearance of the clinical manifestations of Inflammatory Disease or Repiratory Disease. Typical clinical manifestations are coughing, restricted breathing, obstructed airways, fever, chills, tachycardia, tachypnea, altered mental state, hypothermia, hyperthermia, accelerated or repressed breathing or heart rates, increased or decreased white blood cell count, and hypotension. For Respiratory Disease diagnostic tests such as roetgenographic examination, bronchoscopy, lung biopsy, spirography (lung capacity, residual volume, flow rates, etc.) are used. These and other symptoms and diagnostic techniques are well known in the art as set out in standard references such as, Harrison's Principles of Internal Medicine (ISBN 0-07-032370-4) 1994.

The decision to determine the length of therapy may be supported by standard clinical laboratory results from commercially available assays or instrumentation supporting the eradication of the symptoms defining Inflammatory or Respiratory Diseases. The method of the invention may be practiced by continuously or intermittently administering a therapeutically effective dose of the essential an sPLA₂ inhibitor and neutrophil elastase inhibitor ingredients for as long as deemed efficacious for the treatment of the septic episode. The administration can be conducted for up to a total of about 60 days with a preferred course of therapy lasting for up to 14 days.

The decision to terminate may also be based upon the measurement of the patient's baseline protein C levels returning to a value within the range of normal.

The therapy may be restarted upon the return of the Inflammatory or Respiratory disease. The combination therapy of an sPLA₂ inhibitor and a neutrophil elastase inhibitor is also a safe and effective treatment in the prevention and treatment of pediatric forms of Disease.

While the present invention has been illustrated above by certain specific embodiments, it is not intended that these specific examples should limit the scope of the invention as described in the appended claims.

I claim:

1. A pharmaceutical composition comprising:
a neutrophil elastase inhibitor and an sPLA2 inhibitor.

2. A pharmaceutical composition of claim 1 wherein the neutrophil elastase inhibitor is represented by formula (I) 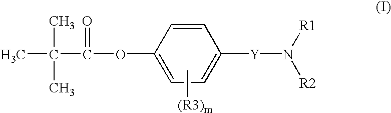
wherein Y represents sulfonyl (—SO₂—) or carbonyl;
(i) R1 and R2 which may be the same or different, each represent
(1) hydrogen,
(2) an alkyl of up to 16 carbon atoms or an alkyl of up to 16 carbon atoms substituted by carboxy,
(3) a group of the formula: 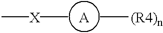
wherein
X represents a single-bond, sulfonyl (—SO₂—), an alkylene of up to 4 carbon atoms, or an alkylene of up to 4 carbon atoms substituted by —COOH or benzyloxy-carbonyl 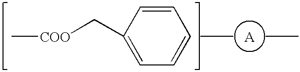
represents a carbocyclic ring or a heterocyclic ring, n represents an integer of 1 to 5,
R4 which may be the same or different represents,
(1) hydrogen or an alkyl group of up to 8 carbon atoms,
(2) an alkoxy of up to 14 carbon atoms,
(3) an alkylthio of up to 6 carbon atoms,
(4) hydroxy, halogen, nitro or trihalomethyl,
(5) a group of the formula: —NR41R42 wherein R41 and R42, which may be the same or different, each represents hydrogen or alkyl of up to 4 carbon atoms,
(6) tetrazole,
(7) sulfonic acid (—SO₃H) or hydroxymethyl (—CH₂OH),
(8) a group of the formula: —SO₂NR41R42 wherein R41 and R42 have the same meanings as described hereinbefore,
(9) a group of the formula: —Z41-COOR43 wherein Z41 represents a single-bond, an alkylene of up to 4 carbon atoms, or an alkenylene of from 2 to 4 carbon atoms, R43 represents hydrogen, an alkyl of up to 4 carbon atoms or benzyl,
(10) a group of the formula: —CONR41R42 wherein R41 and R42 have the same meanings as described hereinbefore,
(11) a group of the formula: —COO-Z42COOR43 wherein Z42 represents an alkylene of up to 4 carbon atoms, R43 represents hydrogen or an alkyl of up to 4 carbon atoms,
(12) a group of the formula: —COO-Z42-CONR41R42 wherein Z42, R41 and R42 have the same meanings as described hereinbefore,
(13) a group of the formula: —OCO-R45 wherein R45 represents an alkyl of up to 8 carbon atoms or p-guanidinophenyl,
(14) a group of the formula: —CO-R46 wherein R46 represents an alkyl of up to 4 carbon atoms,
(15) a group of the formula: —O-Z43-COOR45 wherein Z43 represents an alkylene of up to 6 carbon atoms, R45 represents a hydrogen atom, an alkyl group of up to 8 carbon atoms or a p-guanidinophenyl group,
(16) a group of the formula: 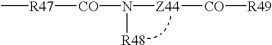
wherein —N-Z44-CO represents an amino acid residue, R48 represents hydrogen or alkyl of up to 4 carbon atoms, and R49 represents hydroxy, alkoxy of up to 4 carbon atoms, amino unsubstituted or substituted by one or two alkyls of up to 4 carbon atoms, carbamoylmethoxy unsubstituted or substituted by one or two alkyls of up to 4 carbon atoms at nitrogen of carbamoyl, R47 represents a single-bond or an alkyl of up to 4 carbon atoms, or 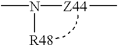
represents a heterocyclic ring containing 3 to 6 carbon atoms and R47 and R49 each has the same meaning as described hereinbefore,
(ii) R1, R2 and nitrogen bonded to R1 and R2 together represent a heterocyclic ring containing at least one nitrogen and substituted by —COOH, or an unsubstituted heterocyclic ring containing at least one nitrogen, R3 represents
(1) hydrogen,
(2) hydroxy,
(3) an alkyl of up to 6 carbon atoms,
(4) halogen,
(5) an alkoxy of up to 4 carbon atoms,
(6) an acyloxy of 2 to 5 carbon atoms, m represents an integer of up to 4,
with the proviso that (1) when R1 and R2 represent hydrogen atom or alkyl group of up to 16 carbon atoms, and R3 represents a hydrogen atom or an alkyl group of up to 6 carbon atoms, Y represents carbonyl (—CO—), and that (2) the compounds wherein one of R1 and R2 represents hydrogen or an alkyl group of up to 16 carbon atoms or 2-carboxyethyl and the other of R1 and R2 represents a group of the formula: 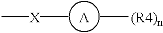
wherein X has the same meaning as described hereinbefore, 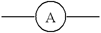
represents a pyridine or pyrrole ring, n represents an integer of 1 or 2, R4 which may be the same or different represents a hydrogen, an alkyl group of up to 8 carbon atoms or a group of the formula: —Z41-COOR43 wherein Z41 and R43 have the same meaning as described hereinbefore, m represents an integer of 1 or 2 and Y and R3 have the same meaning as described hereinbefore, are excluded, or pharmaceutically acceptable salts thereof.

3. A pharmaceutical composition of claim 1 wherein the neutrophil elastase inhibitor selected from the group consisting of:
N-[o-(p-pivaloyloxybenzene)sulfonylaminobenzoyl]glycine,
N-[2-(p-pivaloyloxybenzene)sulfonylamino-5-chlorobenzoyl]glycine,
N-[5-methylthio-2-(p-pivaloyloxybenzene)sulfonylaminobenzoyl]glycine,
N-[2-(p-pivaloyloxybenzene)sulfonylamino-5-propylthiobenzoyl]glycine,
N-[5-methyl-2-(p-pivaloyloxybenzene)sulfonylaminobenzoyl]glycine, and
N-[o-(p-pivaloyloxybenzene)sulfonylaminobenzoyl]glycine methylester,
N-[o-(3-methyl-4-pivaloyloxybenzene)sulfonylaminobenzoyl]-d 1-alanine,
N-[o-(3-methyl-4-pivaloyloxybenzene)sulfonylaminobenzoyl]-beta-alanine,
N-[o-(e-methyl-4-pivaloyloxybenzene)sulfonylaminobenzoyl]-1-alanine,
N-[5-chloro-2-(3-methyl-4-pivaloyloxybenzene)sulfonylaminobenzoyl]-1-alanine and
N-[5-chloro-2-(3-methyl-4-pivaloyloxybenzene)sulfonylamino-benzoyl]-beta -alanine.

4. A pharmaceutical composition of claim 1 wherein the neutrophil elastase inhibitor is N-{o-(p-pivaloyloxybenzene) sulfonylaminobenzoyl)glycine or salts, hydrated salts, or prodrug derivatives thereof.

5. The pharmaceutical composition of claims 1 wherein the weight ratio (a):(b) of (a) neutrophil elastase inhibitor and (b) an sPLA₂ inhibitor is 100:1 to 1:100

6. The pharmaceutical composition of claims 1 wherein the weight of (a) neutrophil elastase inhibitor is in the range of from 1 mg to 5000 mg and the weight of (b) an sPLA₂ inhibitor in the range of 1 to 2000 milligrams.

7. A pharmaceutical composition of claim 1 wherein the sPLA₂ inhibitor is selected from the group of 1H-indole-3-glyoxylamide compounds consisting of the following:
(A) [[3-(2-Amino-1,2-dioxoethyl)-2-methyl-1-(phenylmethyl)-1H-indol-4-yl]oxy]acetic acid,
(B) dl-2-[[3-(2-Amino-1,2-dioxoethyl)-2-methyl-1-(phenylmethyl)-1H-indol-4-yl]oxy]propanoic acid,
(C) [[3-(2-Amino-1,2-dioxoethyl)-1-([1,1′-biphenyl]-2-ylmethyl)-2-methyl-1H-indol-4-yl]oxy]acetic acid,
(D) [[3-(2-Amino-1,2-dioxoethyl)-1-([1,1′-biphenyl]-3-ylmethyl)-2-methyl-1H-indol-4-yl]oxy]acetic acid,
(E) [[3-(2-Amino-1,2-dioxoethyl)-1-([1,1′-biphenyl]-4-ylmethyl)-2-methyl-1H-indol-4-yl]oxy]acetic acid,
(F) [[3-(2-Amino-1,2-dioxoethyl)-1-[(2,6-dichlorophenyl)methyl]-2-methyl-1H-indol-4-yl]oxy]acetic acid
(G) [[3-(2-Amino-1,2-dioxoethyl)-1-[4(-fluorophenyl)methyl]-2-methyl-1H-indol-4-yl]oxy]acetic acid,
(H) [[3-(2-Amino-1,2-dioxoethyl)-2-methyl-1-[(1-naphthalenyl)methyl]-1H-indol-4-yl]oxy]acetic acid,
(I) [[3-(2-Amino-1,2-dioxoethyl)-2-ethyl-1-(phenylmethyl)-1H-indol-4-yl]oxy]acetic acid,
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid,
(L) [[3-(2-amino-1,2-dioxoethyl)-1-([1,1′-biphenyl]-2-ylmethyl)-2-propyl-1H-indol-4-yl]oxy]acetic acid,
(M) [[3-(2-Amino-1,2-dioxoethyl)-2-cyclopropyl-1-(phenylmethyl)-1H-indol-4-yl]oxy]acetic acid,
(N) [[3-(2-Amino-1,2-dioxoethyl)-1-([1,1′-biphenyl]-2-ylmethyl)-2-cyclopropyl-1H-indol-4-yl]oxy]acetic acid,
(O) 4-[[3-(2-Amino-1,2-dioxoethyl)-2-ethyl-1-(phenylmethyl)-1H-indol-5-yl)oxy]butanoic acid,
or any pharmaceutially acceptable salt or prodrug derivative thereof.

8. A pharmaceutical composition of claim 1 wherein the sPLA2 inhibitor is selected from the group of carbazole compounds consisting of the following:
9-benzyl-5,7-dimethoxy-1,2,3,4-tetrahydrocarbazole-4-carboxylic acid hydrazide;
9-benzyl-5,7-dimethoxy-1,2,3,4-tetrahydrocarbazole-4-carboxamide;
[9-benzyl-4-carbamoyl-7-methoxy-1,2,3,4-tetrahydrocarbazol-5-yl]oxyacetic acid sodium salt;
[9-benzyl-4-carbamoyl-7-methoxycarbazol-5-yl]oxyacetic acid;
methyl [9-benzyl-4-carbamoyl-7-methoxycarbazol-5-yl]oxyacetic acid;
9-benzyl-7-methoxy-5-cyanomethyloxy-1,2,3,4-tetrahydrocarbazole-4-carboxamide;
9-benzyl-7-methoxy-5-(1H-tetrazol-5-yl-methyl)oxy)-1,2,3,4-tetrahydrocarbazole-4-carboxamide;
{9-[(phenyl)methyl]-5-carbamoyl-2-methyl-carbazol-4-yl}oxyacetic acid;
{9-[(3-fluorophenyl)methyl]-5-carbamoyl-2-methyl-carbazol-4-yl}oxyacetic acid;
{9-[(3-methylphenyl)methyl]-5-carbamoyl-2-methyl-carbazol-4-yl}oxyacetic acid;
{9-[(phenyl)methyl]-5-carbamoyl-2-(4-trifluoromethylphenyl)-carbazol-4-yl}oxyacetic acid;
9-benzyl-5-(2-methanesulfonamido)ethyloxy-7-methoxy-1,2,3,4-tetrahydrocarbazole-4-carboxamide;
9-benzyl-4-(2-methanesulfonamido)ethyloxy-2-methoxycarbazole-5-carboxamide;
9-benzyl-4-(2-trifluoromethanesulfonamido)ethyloxy-2-methoxycarbazole-5-carboxamide;
9-benzyl-5-methanesulfonamidoylmethyloxy-7-methoxy-1,2,3,4-tetrahydrocarbazole-4-carboxamide;
9-benzyl-4-methanesulfonamidoylmethyloxy-carbazole-5-carboxamide;
[5-carbamoyl-2-pentyl-9-(phenylmethyl)carbazol-4-yl]oxyacetic acid;
[5-carbamoyl-2-(1-methylethyl)-9-(phenylmethyl)carbazol-4-yl]oxyacetic acid;
[5-carbamoyl-9-(phenylmethyl)-2-[(tri(-1-methylethyl)silyl)oxymethyl]carbazol-4-yl]oxyacetic acid;
[5-carbamoyl-2-phenyl-9-(phenylmethyl)carbazol-4-yl]oxyacetic acid[5-carbamoyl-2-(4-chlorophenyl)-9-(phenylmethyl)carbazol-4-yl]oxyacetic acid;
[5-carbamoyl-2-(2-furyl)-9-(phenylmethyl)carbazol-4-yl]oxyacetic acid;
[5-carbamoyl-9-(phenylmethyl)-2-[(tri(-1-methylethyl)silyl)oxymethyl]carbazol-4-yl]oxyacetic acid, lithium salt;
{9-[(phenyl)methyl]-5-carbamoylcarbazol-4-yl}oxyacetic acid;
{9-[(3-fluorophenyl)methyl]-5-carbamoylcarbazol-4-yl}oxyacetic acid;
{9-[(3-phenoxyphenyl)methyl]-5-carbamoylcarbazol-4-yl}oxyacetic acid;
{9-[(2-Fluorophenyl)methyl]-5-carbamoylcarbazol-4-yl}oxyacetic acid;
{9-[(2-trifluoromethylphenyl)methyl]-5-carbamoylcarbazol-4-yl}oxyacetic acid;
{9-[(2-benzylphenyl)methyl]-5-carbamoylcarbazol-4-yl}oxyacetic acid;
{9-[(3-trifluoromethylphenyl)methyl]-5-carbamoylcarbazol-4-yl}oxyacetic acid;
{9-[(1-naphthyl)methyl]-5-carbamoylcarbazol-4-yl}oxyacetic acid;
{9-[(2-cyanophenyl)methyl]-5-carbamoylcarbazol-4-yl}oxyacetic acid;
{9-[(3-cyanophenyl)methyl]-5-carbamoylcarbazol-4-yl}oxyacetic acid;
{9-[(2-methylphenyl)methyl]-5-carbamoylcarbazol-4-yl}oxyacetic acid;
{9-[(3-methylphenyl)methyl]-5-carbamoylcarbazol-4-yl}oxyacetic acid;
{9-[(3,5-dimethylphenyl)methyl]-5-carbamoylcarbazol-4-yl}oxyacetic acid;
{9-[(3-iodophenyl)methyl]-5-carbamoylcarbazol-4-yl}oxyacetic acid;
{9-[(2-Chlorophenyl)methyl]-5-carbamoylcarbazol-4-yl}oxyacetic acid;
{9-[(2,3-difluorophenyl)methyl]-5-carbamoylcarbazol-4-yl}oxyacetic acid;
{9-[(2,6-difluorophenyl)methyl]-5-carbamoylcarbazol-4-yl}oxyacetic acid;
{9-[(2,6-dichlorophenyl)methyl]-5-carbamoylcarbazol-4-yl}oxyacetic acid;
{9-[(3-trifluoromethoxyphenyl)methyl]-5-carbamoylcarbazol-4-yl}oxyacetic acid;
{9-[(2-biphenyl)methyl]-5-carbamoylcarbazol-4-yl}oxyacetic acid;
{9-[(2-Biphenyl)methyl]-5-carbamoylcarbazol-4-yl}oxyacetic acid;
the {9-[(2-Biphenyl)methyl]-5-carbamoylcarbazol-4-yl}oxyacetic acid;
[9-Benzyl-4-carbamoyl-1,2,3,4-tetrahydrocarbaole-5-yl]oxyacetic acid;
{9-[(2-Pyridyl)methyl]-5-carbamoylcarbazol-4-yl}oxyacetic acid;
{9-[(3-Pyridyl)methyl]-5-carbamoylcarbazol-4-yl}oxyacetic acid;
[9-benzyl-4-carbamoyl-8-methyl-1,2,3,4-tetrahydrocarbazol-5-yl]oxyacetic acid;
[9-benzyl-5-carbamoyl-1-methylcarbazol-4-yl]oxyacetic acid;
[9-benzyl-4-carbamoyl-8-fluoro-1,2,3,4-tetrahydrocarbazol-5-yl]oxyacetic acid;
[9-benzyl-5-carbamoyl-1-fluorocarbazol-4-yl]oxyacetic acid;
[9-benzyl-4-carbamoyl-8-chloro-1,2,3,4-tetrahydrocarbazol-5-yl]oxyacetic acid;
[9-benzyl-5-carbamoyl-1-chlorocarbazol-4-yl]oxyacetic acid;
[9-[(Cyclohexyl)methyl]-5-carbamoylcarbazol-4-yl]oxyacetic acid;
[9-[(Cyclopentyl)methyl]-5-carbamoylcarbazol-4-yl]oxyacetic acid;
5-carbamoyl-9-(phenylmethyl)-2-[[(propen-3-yl)oxy]methyl]carbazol-4-yl]oxyacetic acid;
[5-carbamoyl-9-(phenylmethyl)-2-[(propyloxy)methyl]carbazol-4-yl]oxyacetic acid;
9-benzyl-7-methoxy-5-((carboxamidomethyl)oxy)-1,2,3,4-tetrahydrocarbazole-4-carboxamide;
9-benzyl-7-methoxy-5-cyanomethyloxy-carbazole-4-carboxamide;
9-benzyl-7-methoxy-5-((1H-tetrazol-5-yl-methyl)oxy)-carbazole-4-carboxamide;
9-benzyl-7-methoxy-5-((carboxamidomethyl)oxy)-carbazole-4-carboxamide; and
[9-Benzyl-4-carbamoyl-1,2,3,4-tetrahydrocarbaole-5-yl]oxyacetic acid
or a pharmaceutically acceptable racemate, solvate, tautomer, optical isomer, prodrug derivative or salt, thereof.

9. The pharmaceutical composition of claims 1 comprising a suitable carrier, diluent or excipient therefor.

10. A method for the treatment or prevention of Inflammatory Disease comprising administering within a therapeutically effective interval to a mammal in need thereof, therapeutically effective amounts of; a neutrophil elastase inhibitor, and an sPLA₂ inhibitor.

11. A method for the treatment or prevention of Respiratory Disease comprising administering within a therapeutically effective interval to a mammal in need thereof, therapeutically effective amounts of; a neutrophil elastase inhibitor, and an sPLA₂ inhibitor.

12. A method for treatment of a mammal to alleviate or prevent the pathological effects of Respiratory Disease, said method comprising administering to said mammal a therapeutically effective combination of an SPLA₂ inhibitor and a neutrophil elastase inhibitor represented by formula (I) 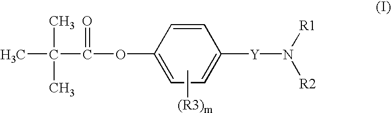
wherein Y represents sulfonyl (—SO₂—) or carbonyl;
(i) R1 and R2 which may be the same or different, each represent
(1) hydrogen,
(2) an alkyl of up to 16 carbon atoms or an alkyl of up to 16 carbon atoms substituted by carboxy,
(3) a group of the formula: 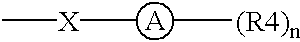
wherein
X represents a single-bond, sulfonyl (—SO₂—), an alkylene of up to 4 carbon atoms, or an alkylene of up to 4 carbon atoms substituted by —COOH or benzyloxy-carbonyl 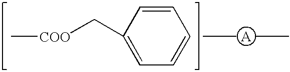
represents a carbocyclic ring or a heterocyclic ring, n represents an integer of 1 to 5,
R4 which may be the same or different represents,
(1) hydrogen or an alkyl group of up to 8 carbon atoms,
(2) an alkoxy of up to 14 carbon atoms,
(3) an alkylthio of up to 6 carbon atoms,
(4) hydroxy, halogen, nitro or trihalomethyl,
(5) a group of the formula: —NR41R42 wherein R41 and R42, which may be the same or different, each represents hydrogen or alkyl of up to 4 carbon atoms,
(6) tetrazole,
(7) sulfonic acid (—SO₃H) or hydroxymethyl (—CH₂OH),
(8) a group of the formula: —SO₂NR41R42 wherein R41 and R42 have the same meanings as described hereinbefore,
(9) a group of the formula: —Z41-COOR43 wherein Z41 represents a single-bond, an alkylene of up to 4 carbon atoms, or an alkenylene of from 2 to 4 carbon atoms, R43 represents hydrogen, an alkyl of up to 4 carbon atoms or benzyl,
(10) a group of the formula: —CONR41R42 wherein R41 and R42 have the same meanings as described hereinbefore,
(11) a group of the formula: —COO-Z42COOR43 wherein Z42 represents an alkylene of up to 4 carbon atoms, R43 represents hydrogen or an alkyl of up to 4 carbon atoms,
(12) a group of the formula: —COO-Z42-CONR41R42 wherein Z42, R41 and R42 have the same meanings as described hereinbefore,
(13) a group of the formula: —OCO-R45 wherein R45 represents an alkyl of up to 8 carbon atoms or p-guanidinophenyl,
(14) a group of the formula: —CO-R46 wherein R46 represents an alkyl of up to 4 carbon atoms,
(15) a group of the formula: —O-Z43-COOR45 wherein Z43 represents an alkylene of up to 6 carbon atoms, R45 represents a hydrogen atom, an alkyl group of up to 8 carbon atoms or a p-guanidinophenyl group,
(16) a group of the formula: 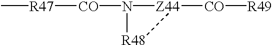
wherein —N-Z44-CO represents an amino acid residue, R48 represents hydrogen or alkyl of up to 4 carbon atoms, and R49 represents hydroxy, alkoxy of up to 4 carbon atoms, amino unsubstituted or substituted by one or two alkyls of up to 4 carbon atoms, carbamoylmethoxy unsubstituted or substituted by one or two alkyls of up to 4 carbon atoms at nitrogen of carbamoyl, R47 represents a single-bond or an alkyl of up to 4 carbon atoms, or 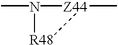
represents a heterocyclic ring containing 3 to 6 carbon atoms and R47 and R49 each has the same meaning as described hereinbefore,
(ii) R1, R2 and nitrogen bonded to R1 and R2 together represent a heterocyclic ring containing at least one nitrogen and substituted by —COOH, or an unsubstituted heterocyclic ring containing at least one nitrogen, R3 represents
(1) hydrogen,
(2) hydroxy,
(3) an alkyl of up to 6 carbon atoms,
(4) halogen,
(5) an alkoxy of up to 4 carbon atoms,
(6) an acyloxy of 2 to 5 carbon atoms, m represents an integer of up to 4,
with the proviso that (1) when R1 and R2 represent hydrogen atom or alkyl group of up to 16 carbon atoms, and R3 represents a hydrogen atom or an alkyl group of up to 6 carbon atoms, Y represents carbonyl (—CO—), and that (2) the compounds wherein one of R1 and R2 represents hydrogen or an alkyl group of up to 16 carbon atoms or 2-carboxyethyl and the other of R1 and R2 represents a group of the formula: 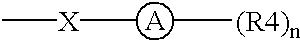
wherein X has the same meaning as described hereinbefore, 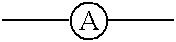
represents a pyridine or pyrrole ring, n represents an integer of 1 or 2, R4 which may be the same or different represents a hydrogen, an alkyl group of up to 8 carbon atoms or a group of the formula: —Z41-COOR43 wherein Z41 and R43 have the same meaning as described hereinbefore, m represents an integer of 1 or 2 and Y and R3 have the same meaning as described hereinbef ore, are excluded, or pharmaceutically acceptable salts thereof.

13. The method according to claim 12 wherein the combination of an sPLA₂ inhibitor and a neutrophil elastase inhibitor is delivered parenterally.

14. The method according to claim 12, wherein the an sPLA₂ inhibitor is administered prior to the neutrophil elastase inhibitor.

15. The method according to claim 15 wherein the neutrophil elastase inhibitor is administered prior to the sPLA₂ inhibitor.

16. Use of the composition of claim 1 for the manufacture of a medicament for treating Inflammatory Disease or Respiratory Disease in a mammal, including a human, currently afflicted with or susceptible to said Diseases.
